(19) United States
(12) Patent Application Publication
(10) Pub. No.: US 2024/0180923 A1
(43) Pub. Date: Jun. 6, 2024

(54) METHODS OF TREATING DISORDERS WITH ULK INHIBITORS

(71) Applicant: Deciphera Pharmaceuticals, LLC, Waltham, MA (US)

(72) Inventors: Rodrigo Ruiz Soto, Waltham, MA (US); Frederic J. Reu, Waltham, MA (US); Matthew L. Sherman, Newton, MA (US); Daniel L. Flynn, Waltham, MA (US); Madhumita Bogdan, Waltham, MA (US); Bryan D. Smith, Waltham, MA (US); Gada Al-Ani, Waltham, MA (US)

(21) Appl. No.: 18/457,825

(22) Filed: Aug. 29, 2023

Related U.S. Application Data
(60) Provisional application No. 63/508,646, filed on Jun. 16, 2023.
(60) Provisional application No. 63/508,652, filed on Jun. 16, 2023.
(60) Provisional application No. 63/495,693, filed on Apr. 12, 2023.
(60) Provisional application No. 63/495,694, filed on Apr. 12, 2023.
(60) Provisional application No. 63/493,825, filed on Apr. 3, 2023.
(60) Provisional application No. 63/493,828, filed on Apr. 3, 2023.
(60) Provisional application No. 63/478,777, filed on Jan. 6, 2023.
(60) Provisional application No. 63/478,782, filed on Jan. 6, 2023.
(60) Provisional application No. 63/403,477, filed on Sep. 2, 2022.
(60) Provisional application No. 63/374,451, filed on Sep. 2, 2022.

(51) Int. Cl. A61K 31/553; A61K 45/06; A61P 35/00

(52) U.S. Cl. CPC A61K 31/553; A61K 45/06; A61P 35/00

(57) ABSTRACT

Described herein are compounds that are inhibitors of autophagy and their use in the treatment of disorders such as cancers.

### CROSS-REFERENCE

This application claims priority to U.S. Provisional Application No. 63/403,477 filed Sep. 2, 2022, U.S. Provisional Application No. 63/374,451 filed Sep. 2, 2022, U.S. Provisional Application No. 63/478,777 filed Jan. 6, 2023, U.S. Provisional Application No. 63/478,782 filed Jan. 6, 2023, U.S. Provisional Application No. 63/493,825 filed Apr. 3, 2023, U.S. Provisional Application No. 63/493,828 filed Apr. 3, 2023, U.S. Provisional Application No. 63/495,693 filed Apr. 12, 2023, U.S. Provisional Application No. 63/495,694 filed Apr. 12, 2023, U.S. Provisional Application No. 63/508,646 filed Jun. 16, 2023, and U.S. Provisional Application No. 63/508,652 filed Jun. 16, 2023, the contents of each of which are incorporated herein by reference in their entireties.

### BACKGROUND

Autophagy (literally meaning “self-eating”) is a process that enables cells to recycle cellular organelles, proteins, stored lipids, glucagon, and other materials for the purpose of generating nutrients under periods of stress. These cellular contents are recycled by engulfment in vesicles called autophagosomes. Autophagosomes subsequently merge with lysosomes that degrade the autophagosomal contents for recycling of nutrients to the cell. Tumor cells are prone to activate autophagy, as these cells have a high metabolic demand, experience cellular stress, and frequently are in hypoxic environments with limited blood flow and nutrient supply. Moreover, chemotherapy and targeted anti-cancer therapies have been shown to induce autophagy as a treatment resistance mechanism, and combination of autophagy inhibition (by genetic loss of function mutations in autophagy genes or by pharmacologic means) with chemotherapeutic regimens has been shown to suppress tumor growth and trigger tumor cell apoptosis to a greater extent than single agent chemotherapy.

Mutant Ras proteins drive approximately 30 percent of all human cancers—including 95 percent of pancreatic cancers, 45 percent of colorectal cancers, and 30% of lung cancers, and treatment of these mutant Ras cancers is currently an area of high unmet medical need. Mutant Ras cancers are highly proliferative and depend on basal levels of autophagy for survival, suggesting that inhibition of autophagy in these “autophagy addicted” cancers is a viable therapeutic approach.

Currently, the most widely used autophagy inhibitors are chloroquine and hydroxychloroquine, which are well-known anti-malarial agents. These anti-malarials have been thought to block autophagy by being sequestered in the lysosomal compartment, raising the pH of these lysosomes and thereby inactivating proteases that degrade and recycle nutrients. These anti-malarial agents have multiple mechanisms of action beyond inhibiting lysosomes and are known to induce retinopathies in patients. Hence there is a need for more targeted agents which selectively block autophagy and do not exhibit the toxicities of these anti-malarial agents. ULK1 kinase is the initiating protein of autophagy and is a serine/threonine kinase. The ULK1 kinase complex is activated in response to cellular stress including nutrient deprivation and energy depletion. Nutrient deprivation activates ULK kinase activity through inhibition of mTORC1, and energy depletion activates ULK kinase activity through activation by AMP-activated protein kinase AMPK. Importantly, kinase dead mutants of ULK kinase block initiation of canonical autophagy, suggesting that small molecule inhibitors of ULK kinase activity would be able to block autophagy.

Further mechanistic studies have shown that genetic deletion of ULK1 inhibits autophagy in cancer cells, relieving FOX3A turn-over and upregulation of the pro-apoptotic protein PUMA. In addition to classical activation of canonical autophagy, ULK1 kinase activity has been shown to be required for Bcl-2-L-13 mediated mitophagy (autophagy of damaged mitochondria). ULK1 and ULK2 kinases have also been demonstrated to rewire cancer cell glucose metabolism which favors increases in the reducing agent NADPH leading to a reduction in toxic reactive oxygen species (ROS). ULK inhibitors may also find utility in blocking these noncanonical pro-tumoral activities of ULK.

Autophagy is also upregulated in host cells and tissues in cancer. Autophagy in pancreatic tissue stellate cells was demonstrated to support tumor growth. Pancreatic stellate cells were shown to support pancreatic cancer tumor metabolism through autophagic alanine secretion. Inhibition of host tissue autophagy was demonstrated to lead to a depletion in circulating arginine (a required amino acid for tumor metabolism and growth) through liver-mediated increases in arginase secretion. Activation of ULK1 kinase was also shown to inactivate the STING pathway in immune cells through inhibitory phosphorylation of STING, mediating a negative feedback mechanism for limiting an innate immune cell response mediated by interferons. Thus, not only is autophagy activated in tumor cells (cancer cell autonomous), but also in other cells in the tumor microenvironment or host tissues (cancer call nonautonomous) to support tumor survival and growth.

Mutant Ras cancers are addicted to autophagy. In pancreatic cancer, mutant Ras signals predominantly through the MAPKAP pathway. Mutant Ras activates RAF kinases, which in turn activate MEK kinases, which finally activate ERK kinases: mutant Ras→RAF→MEK→ERK. Despite mutant Ras signaling through the MAPKAP pathway, inhibitors of this pathway have provided no or little clinical benefit in clinical trials when used as single agents. It has been recently reported that inhibition of the MAPKAP pathway induces autophagy as a compensatory adaptive stress response resistance mechanism. When MEK inhibitors were combined with the autophagy inhibitor hydroxychloroquine, there was synergistic activity leading to regression of a number of mutant Ras or mutant BRAF cancers. Similarly, when ERK inhibitors were combined with the autophagy inhibitor hydroxychloroquine or chloroquine, there was synergistic activity leading to inhibition of mutant Ras pancreatic cancers. It has been demonstrated that genetic depletion of RAF kinases (CRAF and BRAF) led to synergistic antitumor activity in mutant Ras cancer cell lines when autophagy was also genetically depleted. In composite, recent publications highlight that dual inhibition of the RAS/MAPK pathway and the autophagy pathway in mutant Ras cancers is a promising treatment regimen for patients with mutant Ras cancers. It has also been demonstrated that other targeted therapies and chemotherapeutic agents activate tumor autophagy as a resistance mechanism; hence there is rationale for combining such targeted therapeutics or chemotherapeutic agents with inhibitors of autophagy.

It has also been demonstrated that tumor driver receptor tyrosine kinases (RTKs) can modulate autophagy, and that inhibitors of RTKs also activate autophagy through the same mTORC1 and AMP kinase pathways as do inhibitors of mutant RAS or RAF.

There is a need for new targeted therapies which inhibit autophagy and can be used in combination with RTK/RAS/MAPK pathway inhibitors, chemotherapeutic agents, and/or other targeted therapeutics.

### SUMMARY

The present disclosure provides, in part, methods of treating disorders with ULK inhibitors in combination therapies. Described herein, in an embodiment, is a method of treating cancer in a patient in need thereof, comprising: (i) administering to the patient about 20 mg to about 600 mg, once or twice daily, of a compound represented by Formula (I):

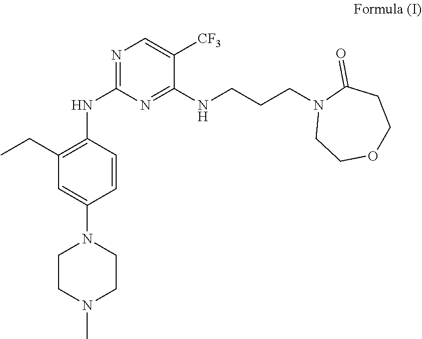 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of one or more additional therapeutic agents.

In another embodiment, described herein is a method of treating cancer in a patient in need thereof, comprising: (i) administering to the patient about 20 mg to about 600 mg, once or twice daily, of a compound represented by Formula (I):

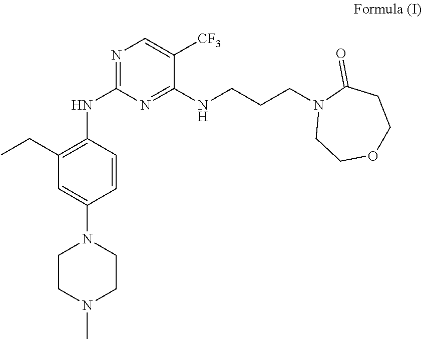 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of trametinib.

In an embodiment, described herein is a method of treating cancer in a patient in need thereof, comprising: (i) administering to the patient about 20 mg to about 600 mg, once or twice daily, of a compound represented by Formula (I):

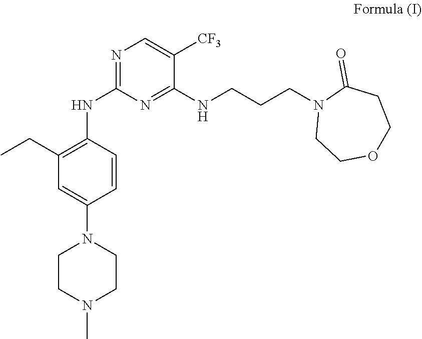 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of binimetinib.

In another embodiment, described herein is a method of treating cancer in a patient in need thereof, comprising administering to the patient: (i) administering to the patient about 20 mg to about 600 mg, once or twice daily, of a compound represented by Formula (I):

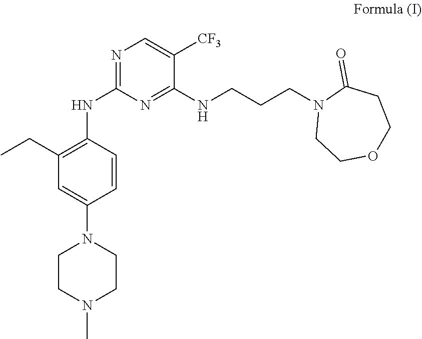 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of a KRAS G12C inhibitor.

In another embodiment, described herein is a method of treating cancer in a patient in need thereof, comprising administering to the patient: (i) orally administering to the patient about 20 mg to about 600 mg, once or twice daily, of a compound represented by Formula (I):

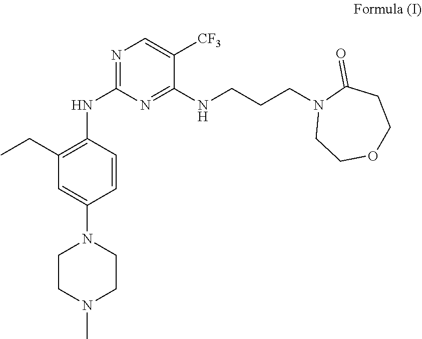 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of sotorasib.

In another embodiment, described herein is a method of treating colorectal cancer in a patient in need thereof, comprising: (i) administering to the patient a therapeutically effective amount of a compound represented by Formula (I):

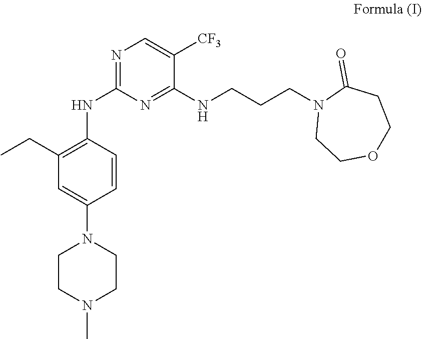 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of encorafenib.

In another embodiment, described herein is a method of treating colorectal cancer in a patient in need thereof, comprising: (i) administering to the patient a therapeutically effective amount of a compound represented by Formula (I):

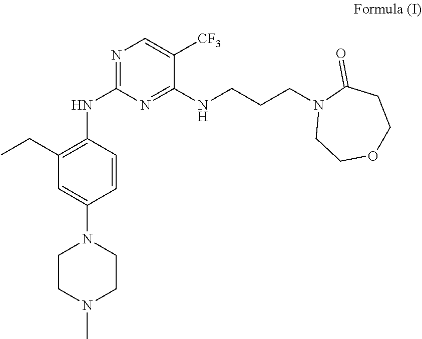 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of encorafenib, and (iii) administering to the patient a therapeutically effective amount of cetuximab.

In another embodiment, described herein a method of treating in a gastrointestinal stromal tumor in a patient in need thereof, comprising administering to the patient: (i) a therapeutically effective amount of an inhibitor of ULK1 kinase, an inhibitor of an ULK2 kinase, or an inhibitor of ULK1 and ULK2 kinases; and (ii) a therapeutically effective amount of ripretinib.

In another embodiment, described herein is a method of treating in an advanced gastrointestinal stromal tumor in a patient in need thereof, comprising: (i) administering to the patient a therapeutically effective amount of a compound represented by Formula (I):

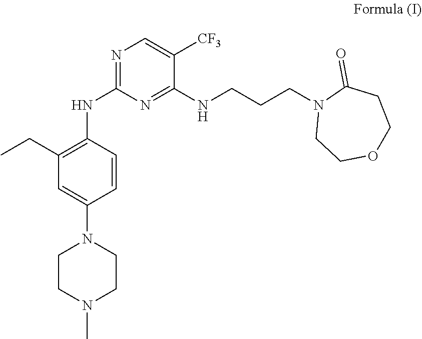 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of ripretinib.

### BRIEF DESCRIPTION OF THE DRAWINGS

FIG. 1 describes the study protocol of Example 1.

FIG. 2 depicts treatment duration and results of response assessments in the monotherapy study of Example 1.

FIG. 3A depicts total and FIG. 3B depicts unbound DCC-3116 area under the curve (AUC) results at Cycle 1 Day 5 (C1D15) in the monotherapy study of Example 1.

FIG. 4 depicts predose trough exposure results evaluated in patients at Cycle 1 Day 5 (C1D15) in the monotherapy study of Example 1.

FIG. 5A depicts trametinib activation of ULK and reversal by DCC-3116 in a KRAS G12C mutated pancreatic cancer (MiaPaca2) cell line. FIG. 5B depicts trametinib activation of autophagy and reversal by DCC-3116 in the MiaPaca2 model. FIG. 5C depicts additivity of DCC-3116 in combo with Trametinib in the MiaPaca2 pancreatic model.

FIG. 6A depicts trametinib activation of ULK and reversal by DCC-3116 in multiple non-small cell lung cancer (NSCLC) cell lines. FIG. 6B depicts trametinib activation of autophagy and reversal by DCC-3116 in the NSCLC lines.

FIG. 7A depicts trametinib activation of ULK and reversal by DCC-3116 in A549* non-small cell lung cancer (NSCLC) cell line. FIG. 7B depicts trametinib activation of autophagy and reversal by DCC-3116 in the NSCLC line.

FIG. 8 depicts study results of DCC-3116 in combination with trametinib in an H358 xenograft model, which demonstrate tumor regressions.

FIG. 9A depicts tumor growth studies of DCC-3116 and binimetinib in an SK-MEL-30 rat study. FIG. 9B depicts tumor growth studies of DCC-3116 and binimetinib in a MiaPaca-2 model.

FIG. 10 depicts DCC-3116 inhibition of binimetinib and ripretinib-induced ULK activity in GIST-T1 cell lines.

FIG. 11A depicts that DCC-3116 reverses ripretinib-induced ULK activation in multiple GIST cell lines. FIG. 11B depicts DCC-3116 inhibits ripreinib-induced autophagy flux in the GIST-T1 cell line. FIG. 11C depicts synergy of DCC-3116 in combination with Ripretinib in the GIST-T1 cancer model. FIG. 11D depicts body weight changes in the GIST T1 xenograft model in relation to combination with DCC-3116 and ripretinib.

FIG. 12A depicts that DCC-3116 reverses KRAS^{G12C} inhibitor (sotorasib or adagrasib)-induced ULK activation in KRAS^{G12C} mutated non-small cell lung cancer. FIG. 12B depicts DCC-3116 inhibits KRAS^{G12C} inhibitor (adagrasib or sotorasib)-induced autophagy flux in KRAS^{G12C} mutated non-small cell lung cancer. FIG. 12C depicts additivity of DCC-3116 in combination with sotorasib in the H358 lung cancer model.

FIGS. 13A, 13B, 13C, and 13D depicts spaghetti plot analyses that demonstrates tumor growth inhibition and/or regression in various combination cohorts at 1 mg/kg (FIG. 13A), 3 mg/kg (FIG. 13B), 10 mg/kg (FIG. 13C), and 30 mg/kg (FIG. 13D) sotorasib.

FIG. 14A depicts KRAS^{G12C} inhibitor activation of ULK and reversal by DCC-3116 in a KRAS^{G12C}-Mutated Pancreatic Cancer (MiaPaCa-2). FIG. 14B depicts KRAS^{G12C} inhibitor activation of autophagy and reversal by DCC-3116 in the KRAS^{G12C}-mutated pancreatic cancer model (MiaPaCa-2). FIG. 14C depicts additivity of DCC-3116 in combination with sotorasib in the MiaPaca2 pancreatic cancer model.

FIG. 15A depicts tumor growth studies of sotorasib in combination with DCC-3116 in a KRAS G12C patient-derived xenograft lung cancer model, (LU11554). FIG. 15B depicts tumor growth studies of adagrasib in combination with DCC-3116 in a patient-derived xenograft lung cancer model, (LU11554).

FIG. 16A depicts combination studies of DCC-3116 and sotorasib in a KRAS^{G12C}-Mutated colorectal cancer cell line (SW1463). FIG. 16B depicts combination studies of DCC-3116 and adagrasib in a KRAS^{G12C}-Mutated colorectal cancer cell line (SW1463).

FIG. 17A depicts combination studies of Compound 1 and MTX-1133, showing induction of ULK activity in the HPAF-II KRAS G12D cell line and inhibition of MRTX-1133 induced ULK activity in the HPAF-II cell line. FIG. 17B depicts combination studies of Compound 1 and MTX-1133, showing inhibition of MRTX1133-induced autophagic flux in the HPAF-II cell line and stable inhibition of MRTX1133 induced autophagy flux by Compound 1 in the HPAF-II cell line.

FIG. 18A depicts pATG13 ELISA assay studies of DCC-3116 and encorafenib and cetuximab in a BRAF^{V600E}-mutated colorectal cancer cell line (HT-29). FIG. 18B depicts autophagy flux assays studies of DCC-3116 and encorafenib and cetuximab in a BRAF^{V600E}-mutated colorectal cancer cell line (HT-29).

FIG. 19 depicts results of DCC-3116 inhibition of encorafenib and cetuximab induced pATG13 in HT-29 tumors.

FIGS. 20A, 20B, 20C, and 20D depict results of various tumor burden studies of DCC-3116 in combination with encorafenib and cetuximab in the BRAF V600E mutated colorectal model, HT-29.

FIG. 21 depicts PK results of sotorasib in combination with DCC-3116 in a MiaPaca-2 PK/PD model.

FIG. 22A depicts plasma concentration results of DCC-3116 in combination with adagrasib in female athymic nude mice (0.5% w/v Na CMC, 0.1% v/v Tween®-80 in water). FIG. 22B depicts plasma concentration results of DCC-3116 in combination with adagrasib in female athymic nude mice (10% Captisol®, 50 mM citrate buffer, pH 5.0).

FIG. 22C depicts PK results of adagrasib in combination with DCC-3116 in nude mice after 5 days of dosing.

FIG. 23 depicts encorafenib and cetuximab induction of ATG13-S³¹⁸ phosphorylation and dose-dependent inhibition by DCC-3116 in the Colo-205 cell line.

FIG. 24 depicts DCC-3116 dose-dependent inhibition of LC3 degradation in cells treated with encorafenib and cetuximab.

FIG. 25 depicts a time course of DCC-3116 inhibition of LC3 degradation in cells treated with encorafenib and cetuximab.

FIG. 26 depicts graphs of tumor growth versus time (study day) with encorafenib and cetuximab and DCC-3116. Drugs were administered on days 6-27 (21 days). Tumor volumes were measured after dosing ended to measure tumor regrowth.

FIG. 27 depicts PK/PD studies of DCC-3116 combined with encorafenib and cetuximab at 2 hours post-dosing on day 7.

### DETAILED DESCRIPTION

### Definitions

A “combination therapy” as used herein is a treatment that includes the administration of two or more therapeutic agents, e.g., a compound of Formula I and an additional therapeutic agent described herein, to a patient in need thereof.

A “RAS/MAPK pathway inhibitor” is an inhibitor of the RAS/MAPK signaling pathway. Inhibitors of this pathway include Ras inhibitors (e.g., AMG-510 (sotorasib), MRTX849 (adagrasib), GDC-6036, MRTX-1133, RMS-9805, RMC-6291, and RMC-6236, and pharmaceutically acceptable salts thereof), RAF inhibitors (e.g., LY3009120, LXH254, RAF709, dabrafenib, vemurafenib, belvarafenib, KIN-2787, and VS-6766, and pharmaceutically acceptable salts thereof), MEK inhibitors (e.g., trametinib, selumetinib, cobimetinib, and binimetinib, and pharmaceutically acceptable salts thereof), and ERK inhibitors (e.g., ulixertinib, SCH772984, LY3214996, ravoxertinib, VX-11e, ERAS-007, and ASTX-029, and pharmaceutically acceptable salts thereof). The terms “RAS/MAPK pathway inhibitor” and “RAS/MAPK kinase inhibitor” are used interchangeably herein.

A “RTK pathway inhibitor” is an inhibitor of the RTK (Receptor Tyrosine Kinase) signaling pathway. Inhibitors of this pathway include KIT inhibitors (e.g., ripretinib, avaprinibavapritinib, sunitinib, and imatinib, and pharmaceutically acceptable salts thereof), EGFR inhibitors (e.g., cetuximab, or pharmaceutically acceptable salts thereof), PDGFRα inhibitors (e.g., JNJ10198409 or a pharmaceutically acceptable salt thereof), VEGFR inhibitors (e.g., regorafenib and pazopanib, and pharmaceutically acceptable salts thereof), BCR-Abl inhibitors (e.g., imatinib, nilotinib, dasatinib, or a pharmaceutically acceptable salt thereof), and an ALK inhibitor (e.g., loralatinb lorlatinib, and alectinib, and pharmaceutically acceptable salts thereof).

“Individual,” “patient,” or “subject” are used interchangeably herein and include any animal, including mammals, including mice, rats, other rodents, rabbits, dogs, cats, swine, cattle, sheep, horses, or primates, and humans. The compounds described herein can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods described herein is desirably a mammal in which treatment of a disorder described herein is desired, such as a human.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts.

As used herein, “treating” includes any effect, e.g., lessening, reducing, modulating, or eliminating, that results in the improvement of the condition, disease, disorder and the like.

As used herein the term “drug holiday” includes a drug holiday of one or more therapeutic agents described herein, e.g., one therapeutic agent described herein, a drug holiday of two therapeutic agents described herein, or a drug holiday of three therapeutic agents described herein. In some embodiments, the drug holiday is a drug holiday of the compound represented by Formula (I). In some embodiments, the drug holiday is a drug holiday of the compound represented by Formula (I) and/or trametinib. In some embodiments, the drug holiday is a drug holiday of the compound represented by Formula (I) and/or binimetinib. In some embodiments, the drug holiday is a drug holiday of the compound represented by Formula (I) and/or sotorasib. In some embodiments, the drug holiday is a drug holiday of the compound represented by Formula (I) and/or adagrasib. In some embodiments, the drug holiday is a drug holiday of the compound represented by Formula (I), encorafenib, and/or cetuximab. In some embodiments, the drug holiday is a drug holiday of the compound represented by Formula (I) and/or ripretinib.

“DCC-3116” as used herein refers to the compound of Formula (I) as defined herein.

As used herein, “Compound A” refers to a compound of the structure:

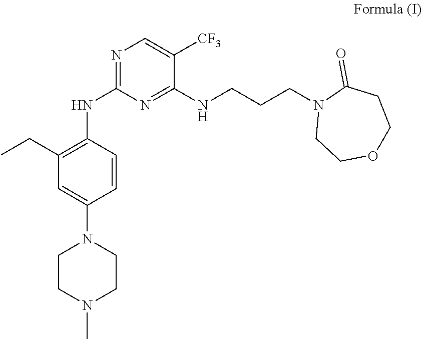 [SMILES: CCc1cc(N2CCN(C)CC2)ccc1Nc1ncc(C(F)(F)F)c(NCCCN2CCOCCC2=O)n1]

“Therapeutically effective amount” includes the amount of the subject compound that will elicit the biological or medical response of a tissue, system, animal or human that is being sought by the researcher, veterinarian, medical doctor or other clinician. A compound described herein, e.g., the compound of Formula (I) is administered in therapeutically effective amounts to treat a condition described herein. Alternatively, a therapeutically effective amount of a compound is the quantity required to achieve a desired therapeutic and/or prophylactic effect, such as an amount which results in the prevention of or a decrease in the symptoms associated with the condition.

A compound described herein can be formulated as a pharmaceutical composition using a pharmaceutically acceptable carrier and administered by a variety of routes. In some embodiments, such compositions are for oral administration. In some embodiments, compositions formulated for oral administration are provided as tablets. In some embodiments, such compositions are for parenteral (by injection) administration. In some embodiments, such compositions are for transdermal administration. In some embodiments, such compositions are for topical administration. In some embodiments, such compositions are for intravenous (IV) administration. In some embodiments, such compositions are for intramuscular (IM) administration. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

### Methods of Treatment

Combinations with One or More Additional Therapeutic Agents

Described herein, in an embodiment, is a method of treating cancer in a patient in need thereof, comprising: (i) administering to the patient about 20 mg to about 1200 mg, daily, of a compound represented by Formula (I):

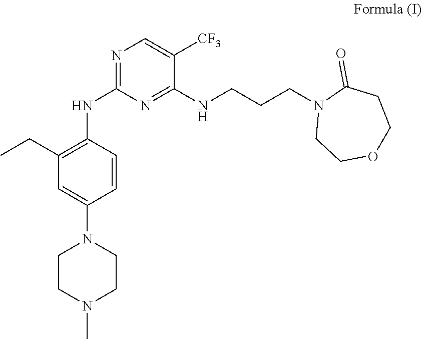 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of one or more additional therapeutic agents. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 300 mg, once daily, of the compound or pharmaceutically acceptable salt thereof. In some embodiments, the one or more additional therapeutic agents is selected from the group consisting of a RTK pathway inhibitor, an EGFR inhibitor, a KIT inhibitor, and a combination thereof. In some embodiments, the EGFR inhibitor is cetuximab. In some embodiments, the KIT inhibitor is ripretinib or a pharmaceutically acceptable salt thereof. In some embodiments, the RAS/MAPK pathway inhibitor is selected from the group consisting of a MEK inhibitor, an ERK inhibitor, a RAF inhibitor, a Ras inhibitor, and a combination thereof. In some embodiments, the MEK inhibitor is selected from the group consisting of trametinib, binimetinib, and pharmaceutically acceptable salts thereof. In some embodiments, the RAF inhibitor is encorafenib, or a pharmaceutically acceptable salt thereof. In some embodiments, the Ras inhibitor is sotorasib, adagrasib, or a pharmaceutically acceptable salt thereof. In some embodiments, the Ras inhibitor is MRTX-1133, or a pharmaceutically acceptable salt thereof. In some embodiments, the one or more additional therapeutic agents is selected from the group consisting of trametinib, binimetinib, sotorasib, adagrasib, cetuximab, encorafenib, ripretinib, and a combination thereof. In some embodiments, the cancer is selected from the group consisting of pancreatic ductal adenocarcinoma, non-small cell lung cancer, colorectal cancer, melanoma, and gastrointestinal stromal tumors.

Described herein, in an embodiment, is a method of treating cancer in a patient in need thereof, comprising: (i) administering to the patient about 20 mg to about 600 mg, once or twice daily, of a compound represented by Formula (I):

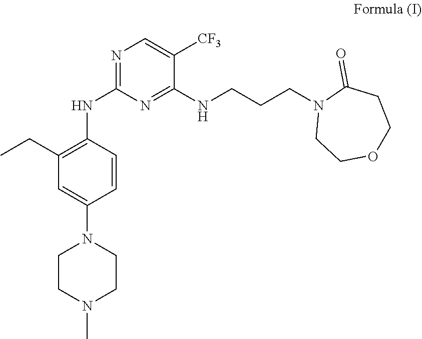 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of one or more additional therapeutic agents. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 300 mg, once daily, of the compound or pharmaceutically acceptable salt thereof. In some embodiments, the one or more additional therapeutic agents is selected from the group consisting of a RTK pathway inhibitor, an EGFR inhibitor, a KIT inhibitor, and a combination thereof. In some embodiments, the EGFR inhibitor is cetuximab. In some embodiments, the KIT inhibitor is ripretinib or a pharmaceutically acceptable salt thereof. In some embodiments, the RAS/MAPK pathway inhibitor is selected from the group consisting of a MEK inhibitor, an ERK inhibitor, a RAF inhibitor, a Ras inhibitor, and a combination thereof. In some embodiments, the MEK inhibitor is selected from the group consisting of trametinib, binimetinib, and pharmaceutically acceptable salts thereof. In some embodiments, the RAF inhibitor is encorafenib, or a pharmaceutically acceptable salt thereof. In some embodiments, the Ras inhibitor is sotorasib or a pharmaceutically acceptable salt thereof. In some embodiments, the Ras inhibitor is adagrasib or a pharmaceutically acceptable salt thereof. In some embodiments, the Ras inhibitor is MRTX-1133 or a pharmaceutically acceptable salt thereof. In some embodiments, the one or more additional therapeutic agents is selected from the group consisting of trametinib, binimetinib, sotorasib, adagrasib, cetuximab, encorafenib, ripretinib, and a combination thereof. In some embodiments, the cancer is selected from the group consisting of pancreatic ductal adenocarcinoma, non-small cell lung cancer, colorectal cancer, melanoma, and gastrointestinal stromal tumors.
Combinations with Trametinib

In another embodiment, described herein is a method of treating cancer in a patient in need thereof, comprising: (i) administering to the patient about 20 mg to about 1200 mg daily, of a compound represented by Formula (I):

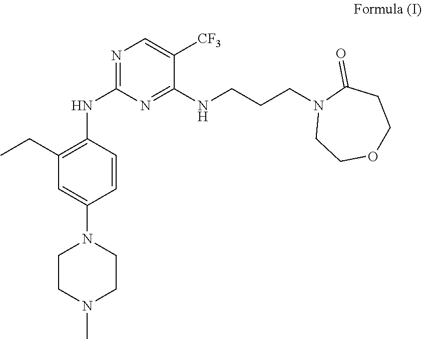 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of trametinib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 0.5 mg to about 2.5 mg, once daily, of trametinib. In some embodiments, the method comprises administering to the patient about 2 mg, once daily, of trametinib. In some embodiments, the method comprises administering to the patient about 1.5 mg, once daily, of trametinib. In some embodiments, the method comprises administering to the patient about 1 mg, once daily, of trametinib. In some embodiments, the method comprises administering to the patient about 0.5 mg to about 2.5 mg, twice daily, of trametinib. In some embodiments, the method comprises administering to the patient about 2 mg, once daily, of trametinib. In some embodiments, the method comprises administering to the patient about 1.5 mg, twice daily, of trametinib. In some embodiments, the method comprises administering to the patient about 1 mg, twice daily, of trametinib. In some embodiments, the cancer is selected from the group consisting of pancreatic ductal adenocarcinoma, non-small cell lung cancer, colorectal cancer, and melanoma. In some embodiments, the pancreatic ductal adenocarcinoma has one or more KRAS mutations. In some embodiments, the non-small cell lung cancer has one or more mutations selected from the group consisting of a RAS mutation, an NF1 mutation, a RAF mutation, and a combination thereof. In some embodiments, the non-small cell lung cancer has one or more RAS mutation wherein the one or more RAS mutation is one or more NRAS mutation, one or more KRAS mutation, one or more HRAS mutation, or a combination thereof. In some embodiments, the non-small cell lung cancer has one or more RAF mutation wherein the one or more RAF mutation is one or more ARAF mutations, one or more BRAF mutations, one or more CRAF mutations, or a combination thereof. In some embodiments, the colorectal cancer has one or more mutations selected from the group consisting of a RAS mutation, an NF1 mutation, a RAF mutation, and a combination thereof. In some embodiments, the colorectal cancer has one or more RAS mutation wherein the one or more RAS mutation is one or more NRAS mutation, one or more KRAS mutation, one or more HRAS mutation, or a combination thereof. In some embodiments, the colorectal cancer has one or more RAF mutation wherein the one or more RAF mutation is one or more ARAF mutations, one or more BRAF mutations, one or more CRAF mutations, or a combination thereof. In some embodiments, the melanoma has one or more RAS mutations. In some embodiments, the melanoma has one or more RAS mutation wherein the one or more RAS mutation is one or more NRAS mutation, one or more KRAS mutation, one or more HRAS mutation, or a combination thereof. In some embodiments, the melanoma has one or more NRAS mutations. In some embodiments, the melanoma is an unresectable or metastatic melanoma. In some embodiments, a patient having a RAF-mutant non-small cell lung cancer was previously administered at least one prior therapy. In some embodiments, a patient having a RAF-mutant non-small cell lung cancer was previously administered at least three (e.g., three to five) prior therapies. In some embodiments, a patient having a NF1-mutant non-small cell lung cancer was previously administered at least one prior therapy. In some embodiments, a patient having an NF1-mutant non-small cell lung cancer was previously administered at least three (e.g., three to five) prior therapies. In some embodiments, a patient having a RAS-mutant non-small cell lung cancer was previously administered at least one prior therapy. In some embodiments, a patient having a RAS-mutant non-small cell lung cancer was previously administered at least three (e.g., three to five) prior therapies. In some embodiments, a patient having a RAF-mutant colorectal cancer was previously administered at least one prior therapy. In some embodiments, a patient having a RAF-mutant colorectal cancer was previously administered at least three (e.g., three to four) prior therapies. In some embodiments, a patient having a NF1-mutant colorectal cancer was previously administered at least one prior therapy. In some embodiments, a patient having an NF1-mutant colorectal cancer was previously administered at least three (e.g., three to four) prior therapies. In some embodiments, a patient having a RAS-mutant colorectal cancer was previously administered at least one prior therapy. In some embodiments, a patient having a RAS-mutant colorectal cancer was previously administered at least three (e.g., three to four) prior therapies. In some embodiments, a patient having an NRAS-mutant melanoma was previously administered at least one prior therapy. In some embodiments, a patient having an NRAS-mutant melanoma was previously administered at least two (e.g., two to three) prior therapies.

In another embodiment, described herein is a method of treating cancer in a patient in need thereof, comprising: (i) administering to the patient about 20 mg to about 600 mg, once or twice daily, of a compound represented by Formula (I):

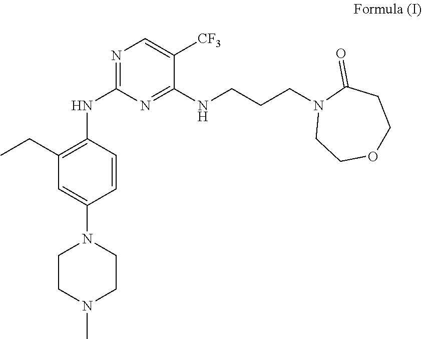 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of trametinib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 0.5 mg to about 2.5 mg, once daily, of trametinib. In some embodiments, the method comprises administering to the patient about 2 mg, once daily, of trametinib. In some embodiments, the method comprises administering to the patient about 0.5 mg to about 2.5 mg, twice daily, of trametinib. In some embodiments, the method comprises administering to the patient about 2 mg, once daily, of trametinib. In some embodiments, the method comprises administering to the patient about 1.5 mg, twice daily, of trametinib. In some embodiments, the method comprises administering to the patient about 1 mg, twice daily, of trametinib. In some embodiments, the cancer is selected from the group consisting of pancreatic ductal adenocarcinoma, non-small cell lung cancer, colorectal cancer, and melanoma. In some embodiments, the pancreatic ductal adenocarcinoma has one or more KRAS mutations. In some embodiments, the non-small cell lung cancer has one or more mutations selected from the group consisting of a RAS mutation, an NF1 mutation, a RAF mutation, and a combination thereof. In some embodiments, the non-small cell lung cancer has one or more RAS mutation wherein the one or more RAS mutation is one or more NRAS mutation, one or more KRAS mutation, one or more HRAS mutation, or a combination thereof. In some embodiments, the non-small cell lung cancer has one or more RAF mutation wherein the one or more RAF mutation is one or more ARAF mutations, one or more BRAF mutations, one or more CRAF mutations, or a combination thereof. In some embodiments, the colorectal cancer has one or more mutations selected from the group consisting of a RAS mutation, an NF1 mutation, a RAF mutation, or a combination thereof. In some embodiments, the colorectal cancer has one or more RAS mutation wherein the one or more RAS mutation is one or more NRAS mutation, one or more KRAS mutation, one or more HRAS mutation, or a combination thereof. In some embodiments, the colorectal cancer has one or more RAF mutation wherein the one or more RAF mutation is one or more ARAF mutations, one or more BRAF mutations, one or more CRAF mutations, or a combination thereof. In some embodiments, the melanoma has one or more RAS mutations. In some embodiments, the melanoma has one or more RAS mutation wherein the one or more RAS mutation is one or more NRAS mutation, one or more KRAS mutation, one or more HRAS mutation, or a combination thereof. In some embodiments, the melanoma has one or more NRAS mutations. In some embodiments, the melanoma is an unresectable or metastatic melanoma. In some embodiments, a patient having a RAF-mutant non-small cell lung cancer was previously administered at least one prior therapy. In some embodiments, a patient having a RAF-mutant non-small cell lung cancer was previously administered at least three (e.g., three to five) prior therapies. In some embodiments, a patient having a NF1-mutant non-small cell lung cancer was previously administered at least one prior therapy. In some embodiments, a patient having an NF1-mutant non-small cell lung cancer was previously administered at least three (e.g., three to five) prior therapies. In some embodiments, a patient having a RAS-mutant non-small cell lung cancer was previously administered at least one prior therapy. In some embodiments, a patient having a RAS-mutant non-small cell lung cancer was previously administered at least three (e.g., three to five) prior therapies. In some embodiments, a patient having a RAF-mutant colorectal cancer was previously administered at least one prior therapy. In some embodiments, a patient having a RAF-mutant colorectal cancer was previously administered at least three (e.g., three to four) prior therapies. In some embodiments, a patient having a NF1-mutant colorectal cancer was previously administered at least one prior therapy. In some embodiments, a patient having an NF1-mutant colorectal cancer was previously administered at least three (e.g., three to four) prior therapies. In some embodiments, a patient having a RAS-mutant colorectal cancer was previously administered at least one prior therapy. In some embodiments, a patient having a RAS-mutant colorectal cancer was previously administered at least three (e.g., three to four) prior therapies. In some embodiments, a patient having an NRAS-mutant melanoma was previously administered at least one prior therapy. In some embodiments, a patient having an NRAS-mutant melanoma was previously administered at least two (e.g., two to three) prior therapies.

In some embodiments, the method comprises administering to the patient each of the compound represented by Formula (I) and trametinib for from 1 to 30 consecutive days, followed by a drug holiday of the compound represented by Formula (I) and/or trametinib, followed by administering to the patient each of the compound represented by Formula (I) and trametinib for from 1 to 30 consecutive days. In some embodiments, the method comprises administering to the patient each of the compound represented by Formula (I) and trametinib for from 1 to 28 consecutive days, followed by a drug holiday of the compound represented by Formula (I) and/or trametinib, followed by administering to the patient each of the compound represented by Formula (I) and trametinib for from 1 to 28 consecutive days. In some embodiments, the method comprises administering to the patient each of the compound represented by Formula (I) and trametinib for from 1 to 21 consecutive days, followed by a drug holiday of the compound represented by Formula (I) and/or trametinib, followed by administering to the patient each of the compound represented by Formula (I) and trametinib for from 1 to 21 consecutive days. In some embodiments, the method comprises administering to the patient each of the compound represented by Formula (I) and trametinib for from 1 to 14 consecutive days, followed by a drug holiday of the compound represented by Formula (I) and/or trametinib, followed by administering to the patient each of the compound represented by Formula (I) and trametinib for from 1 to 14 consecutive days. In some embodiments, the method comprises administering to the patient each of the compound represented by Formula (I) and trametinib for from 1 to 7 consecutive days, followed by a drug holiday of the compound represented by Formula (I) and/or trametinib, followed by administering to the patient each of the compound represented by Formula (I) and trametinib for from 1 to 7 consecutive days.

In some embodiments, the method comprises administering to the patient each of the compound represented by Formula (I) and trametinib for from 1 to 30 consecutive days, followed by a drug holiday of the compound represented by Formula (I) and/or trametinib, followed by administering to the patient each of the compound represented by Formula (I) and trametinib for from 1 to 7 consecutive days. In some embodiments, the method comprises administering to the patient each of the compound represented by Formula (I) and trametinib for from 1 to 30 consecutive days, followed by a drug holiday of the compound represented by Formula (I) and/or trametinib, followed by administering to the patient each of the compound represented by Formula (I) and trametinib for from 1 to 14 consecutive days. In some embodiments, the method comprises administering to the patient each of the compound represented by Formula (I) and trametinib for from 1 to 30 consecutive days, followed by a drug holiday of the compound represented by Formula (I) and/or trametinib, followed by administering to the patient each of the compound represented by Formula (I) and trametinib for from 1 to 21 consecutive days. In some embodiments, the method comprises administering to the patient each of the compound represented by Formula (I) and trametinib for from 1 to 30 consecutive days, followed by a drug holiday of the compound represented by Formula (I) and/or trametinib, followed by administering to the patient each of the compound represented by Formula (I) and trametinib for from 1 to 28 consecutive days.

In some embodiments, the method comprises administering to the patient each of the compound represented by Formula (I) and trametinib for from 1 to 21 consecutive days, followed by a drug holiday of the compound represented by Formula (I) and/or trametinib, followed by administering to the patient each of the compound represented by Formula (I) and trametinib for from 1 to 7 consecutive days. In some embodiments, the method comprises administering to the patient each of the compound represented by Formula (I) and trametinib for from 1 to 21 consecutive days, followed by a drug holiday of the compound represented by Formula (I) and/or trametinib, followed by administering to the patient each of the compound represented by Formula (I) and trametinib for from 1 to 14 consecutive days. In some embodiments, the method comprises administering to the patient each of the compound represented by Formula (I) and trametinib for from 1 to 21 consecutive days, followed by a drug holiday of the compound represented by Formula (I) and/or trametinib, followed by administering to the patient each of the compound represented by Formula (I) and trametinib for from 1 to 28 consecutive days. In some embodiments, the method comprises administering to the patient each of the compound represented by Formula (I) and trametinib for from 1 to 21 consecutive days, followed by a drug holiday of the compound represented by Formula (I) and/or trametinib, followed by administering to the patient each of the compound represented by Formula (I) and trametinib for from 1 to 30 consecutive days.

In some embodiments, the method comprises administering to the patient each of the compound represented by Formula (I) and trametinib for from 1 to 14 consecutive days, followed by a drug holiday of the compound represented by Formula (I) and/or trametinib, followed by administering to the patient each of the compound represented by Formula (I) and trametinib for from 1 to 7 consecutive days. In some embodiments, the method comprises administering to the patient each of the compound represented by Formula (I) and trametinib for from 1 to 14 consecutive days, followed by a drug holiday of the compound represented by Formula (I) and/or trametinib, followed by administering to the patient each of the compound represented by Formula (I) and trametinib for from 1 to 21 consecutive days. In some embodiments, the method comprises administering to the patient each of the compound represented by Formula (I) and trametinib for from 1 to 14 consecutive days, followed by a drug holiday of the compound represented by Formula (I) and/or trametinib, followed by administering to the patient each of the compound represented by Formula (I) and trametinib for from 1 to 28 consecutive days. In some embodiments, the method comprises administering to the patient each of the compound represented by Formula (I) and trametinib for from 1 to 14 consecutive days, followed by a drug holiday of the compound represented by Formula (I) and/or trametinib, followed by administering to the patient each of the compound represented by Formula (I) and trametinib for from 1 to 30 consecutive days.

In some embodiments, the method comprises administering to the patient each of the compound represented by Formula (I) and trametinib for from 1 to 7 consecutive days, followed by a drug holiday of the compound represented by Formula (I) and/or trametinib, followed by administering to the patient each of the compound represented by Formula (I) and trametinib for from 1 to 14 consecutive days. In some embodiments, the method comprises administering to the patient each of the compound represented by Formula (I) and trametinib for from 1 to 7 consecutive days, followed by a drug holiday of the compound represented by Formula (I) and/or trametinib, followed by administering to the patient each of the compound represented by Formula (I) and trametinib for from 1 to 21 consecutive days. In some embodiments, the method comprises administering to the patient each of the compound represented by Formula (I) and trametinib for from 1 to 7 consecutive days, followed by a drug holiday of the compound represented by Formula (I) and/or trametinib, followed by administering to the patient each of the compound represented by Formula (I) and trametinib for from 1 to 28 consecutive days. In some embodiments, the method comprises administering to the patient each of the compound represented by Formula (I) and trametinib for from 1 to 7 consecutive days, followed by a drug holiday of the compound represented by Formula (I) and/or trametinib, followed by administering to the patient each of the compound represented by Formula (I) and trametinib for from 1 to 30 consecutive days.

In some embodiments, the method comprises administering to the patient each of the compound represented by Formula (I) and trametinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or trametinib, followed by administering to the patient each of the compound represented by Formula (I) and trametinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and trametinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or trametinib, followed by administering to the patient each of the compound represented by Formula (I) and trametinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or trametinib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or trametinib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or trametinib is a period of 1-4 weeks. In some embodiments, the drug holiday of the compound represented by Formula (I) and/or trametinib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of trametinib in the patient. In some embodiments, the drug holiday is about 1-3 times the half-life of trametinib in the patient. In some embodiments, the drug holiday is about 1-4 times the half-life of trametinib in the patient. In some embodiments, the drug holiday is about 1-5 times the half-life of trametinib in the patient. In some embodiments, the drug holiday is about 1-6 times the half-life of trametinib in the patient. In some embodiments, the drug holiday is about 4-5 times the half-life of trametinib in the patient. In some embodiments, the drug holiday is about 4 times the half-life of trametinib in the patient. In some embodiments, the drug holiday is about 5 times the half-life of trametinib in the patient.

Combinations with Binimetinib

In an embodiment, described herein is a method of treating cancer in a patient in need thereof, comprising: (i) administering to the patient about 20 mg to about 1200 mg, daily, of a compound represented by Formula (I):

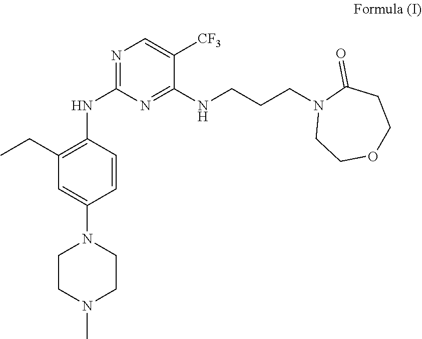 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of binimetinib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 20 mg to about 50 mg, twice daily, of binimetinib. In some embodiments, the method comprises administering to the patient about 45 mg, twice daily, of binimetinib. In some embodiments, the method comprises administering to the patient about 40 mg, twice daily, of binimetinib. In some embodiments, the method comprises administering to the patient about 35 mg, twice daily, of binimetinib. In some embodiments, the method comprises administering to the patient about 30 mg, twice daily, of binimetinib. In some embodiments, the method comprises administering to the patient about 25 mg, twice daily, of binimetinib. In some embodiments, the method comprises administering to the patient about 20 mg, twice daily, of binimetinib. In some embodiments, the method comprises administering to the patient about 20 mg to about 50 mg, once daily, of binimetinib. In some embodiments, the method comprises administering to the patient about 45 mg, once daily, of binimetinib. In some embodiments, the method comprises administering to the patient about 40 mg, once daily, of binimetinib. In some embodiments, the method comprises administering to the patient about 35 mg, once daily, of binimetinib. In some embodiments, the method comprises administering to the patient about 30 mg, once daily, of binimetinib. In some embodiments, the method comprises administering to the patient about 25 mg, once daily, of binimetinib. In some embodiments, the method comprises administering to the patient about 20 mg, once daily, of binimetinib. In some embodiments, the cancer is selected from the group consisting of pancreatic ductal adenocarcinoma, non-small cell lung cancer, colorectal cancer, and melanoma. In some embodiments, the pancreatic ductal adenocarcinoma has one or more KRAS mutations. In some embodiments, the non-small cell lung cancer has one or more mutations selected from the group consisting of a RAS mutation, an NF1 mutation, a RAF mutation, and a combination thereof. In some embodiments, the non-small cell lung cancer has one or more RAS mutation wherein the one or more RAS mutation is one or more NRAS mutation, one or more KRAS mutation, one or more HRAS mutation, or a combination thereof. In some embodiments, the non-small cell lung cancer has one or more RAF mutation wherein the one or more RAF mutation is one or more ARAF mutations, one or more BRAF mutations, one or more CRAF mutations, or a combination thereof. In some embodiments, the colorectal cancer has one or more mutations selected from the group consisting of a RAS mutation, an NF1 mutation, a RAF mutation, and a combination thereof. In some embodiments, the colorectal cancer has one or more RAS mutation wherein the one or more RAS mutation is one or more NRAS mutation, one or more KRAS mutation, one or more HRAS mutation, or a combination thereof. In some embodiments, the colorectal cancer has one or more RAF mutation wherein the one or more RAF mutation is one or more ARAF mutations, one or more BRAF mutations, one or more CRAF mutations, or a combination thereof. In some embodiments, the melanoma has one or more RAS mutations. In some embodiments, the melanoma has one or more RAS mutation wherein the one or more RAS mutation is one or more NRAS mutation, one or more KRAS mutation, one or more HRAS mutation, or a combination thereof. In some embodiments, the melanoma has one or more NRAS mutations. In some embodiments, the melanoma is an unresectable or metastatic melanoma. In some embodiments, a patient having a RAF-mutant non-small cell lung cancer was previously administered at least one prior therapy. In some embodiments, a patient having a RAF-mutant non-small cell lung cancer was previously administered at least three (e.g., three to five) prior therapies. In some embodiments, a patient having a NF1-mutant non-small cell lung cancer was previously administered at least one prior therapy. In some embodiments, a patient having an NF1-mutant non-small cell lung cancer was previously administered at least three (e.g., three to five) prior therapies. In some embodiments, a patient having a RAS-mutant non-small cell lung cancer was previously administered at least one prior therapy. In some embodiments, a patient having a RAS-mutant non-small cell lung cancer was previously administered at least three (e.g., three to five) prior therapies. In some embodiments, a patient having a RAF-mutant colorectal cancer was previously administered at least one prior therapy. In some embodiments, a patient having a RAF-mutant colorectal cancer was previously administered at least three (e.g., three to four) prior therapies. In some embodiments, a patient having a NF1-mutant colorectal cancer was previously administered at least one prior therapy. In some embodiments, a patient having an NF1-mutant colorectal cancer was previously administered at least three (e.g., three to four) prior therapies. In some embodiments, a patient having a RAS-mutant colorectal cancer was previously administered at least one prior therapy. In some embodiments, a patient having a RAS-mutant colorectal cancer was previously administered at least three (e.g., three to four) prior therapies. In some embodiments, a patient having an NRAS-mutant melanoma was previously administered at least one prior therapy. In some embodiments, a patient having an NRAS-mutant melanoma was previously administered at least two (e.g., two to three) prior therapies.

In an embodiment, described herein is a method of treating cancer in a patient in need thereof, comprising: (i) administering to the patient about 20 mg to about 600 mg, once or twice daily, of a compound represented by Formula (I):

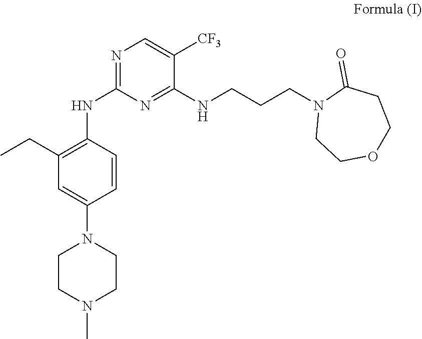 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of binimetinib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 20 mg to about 50 mg, twice daily, of binimetinib. In some embodiments, the method comprises administering to the patient about 45 mg, twice daily, of binimetinib. In some embodiments, the method comprises administering to the patient about 20 mg to about 50 mg, once daily, of binimetinib. In some embodiments, the method comprises administering to the patient about 45 mg, once daily, of binimetinib. In some embodiments, the method comprises administering to the patient about 40 mg, once daily, of binimetinib. In some embodiments, the method comprises administering to the patient about 35 mg, once daily, of binimetinib. In some embodiments, the method comprises administering to the patient about 30 mg, once daily, of binimetinib. In some embodiments, the method comprises administering to the patient about 25 mg, once daily, of binimetinib. In some embodiments, the method comprises administering to the patient about 20 mg, once daily, of binimetinib. In some embodiments, the cancer is selected from the group consisting of pancreatic ductal adenocarcinoma, non-small cell lung cancer, colorectal cancer, and melanoma. In some embodiments, the pancreatic ductal adenocarcinoma has one or more KRAS mutations. In some embodiments, the non-small cell lung cancer has one or more mutations selected from the group consisting of a RAS mutation, an NF1 mutation, a RAF mutation, and a combination thereof. In some embodiments, the non-small cell lung cancer has one or more RAS mutation wherein the one or more RAS mutation is one or more NRAS mutation, one or more KRAS mutation, one or more HRAS mutation, or a combination thereof. In some embodiments, the non-small cell lung cancer has one or more RAF mutation wherein the one or more RAF mutation is one or more ARAF mutations, one or more BRAF mutations, one or more CRAF mutations, or a combination thereof. In some embodiments, the colorectal cancer has one or more mutations selected from the group consisting of a RAS mutation, an NF1 mutation, a RAF mutation, and a combination thereof. In some embodiments, the colorectal cancer has one or more RAS mutation wherein the one or more RAS mutation is one or more NRAS mutation, one or more KRAS mutation, one or more HRAS mutation, or a combination thereof. In some embodiments, the colorectal cancer has one or more RAF mutation wherein the one or more RAF mutation is one or more ARAF mutations, one or more BRAF mutations, one or more CRAF mutations, or a combination thereof. In some embodiments, the melanoma has one or more RAS mutations. In some embodiments, the melanoma has one or more RAS mutation wherein the one or more RAS mutation is one or more NRAS mutation, one or more KRAS mutation, one or more HRAS mutation, or a combination thereof. In some embodiments, the melanoma has one or more NRAS mutations. In some embodiments, the melanoma is an unresectable or metastatic melanoma. In some embodiments, a patient having a RAF-mutant non-small cell lung cancer was previously administered at least one prior therapy. In some embodiments, a patient having a RAF-mutant non-small cell lung cancer was previously administered at least three (e.g., three to five) prior therapies. In some embodiments, a patient having a NF1-mutant non-small cell lung cancer was previously administered at least one prior therapy. In some embodiments, a patient having an NF1-mutant non-small cell lung cancer was previously administered at least three (e.g., three to five) prior therapies. In some embodiments, a patient having a RAS-mutant non-small cell lung cancer was previously administered at least one prior therapy. In some embodiments, a patient having a RAS-mutant non-small cell lung cancer was previously administered at least three (e.g., three to five) prior therapies. In some embodiments, a patient having a RAF-mutant colorectal cancer was previously administered at least one prior therapy. In some embodiments, a patient having a RAF-mutant colorectal cancer was previously administered at least three (e.g., three to four) prior therapies. In some embodiments, a patient having a NF1-mutant colorectal cancer was previously administered at least one prior therapy. In some embodiments, a patient having an NF1-mutant colorectal cancer was previously administered at least three (e.g., three to four) prior therapies. In some embodiments, a patient having a RAS-mutant colorectal cancer was previously administered at least one prior therapy. In some embodiments, a patient having a RAS-mutant colorectal cancer was previously administered at least three (e.g., three to four) prior therapies. In some embodiments, a patient having an NRAS-mutant melanoma was previously administered at least one prior therapy. In some embodiments, a patient having an NRAS-mutant melanoma was previously administered at least two (e.g., two to three) prior therapies.

In some embodiments, the method comprises administering to the patient each of the compound represented by Formula (I) and binimetinib for from 1 to 30 consecutive days, followed by a drug holiday of the compound represented by Formula (I) and/or binimetinib, followed by administering to the patient each of the compound represented by Formula (I) and binimetinib for from 1 to 30 consecutive days. In some embodiments, the method comprises administering to the patient each of the compound represented by Formula (I) and binimetinib for from 1 to 28 consecutive days, followed by a drug holiday of the compound represented by Formula (I) and/or binimetinib, followed by administering to the patient each of the compound represented by Formula (I) and binimetinib for from 1 to 28 consecutive days. In some embodiments, the method comprises administering to the patient each of the compound represented by Formula (I) and binimetinib for from 1 to 21 consecutive days, followed by a drug holiday of the compound represented by Formula (I) and/or binimetinib, followed by administering to the patient each of the compound represented by Formula (I) and binimetinib for from 1 to 21 consecutive days. In some embodiments, the method comprises administering to the patient each of the compound represented by Formula (I) and binimetinib for from 1 to 14 consecutive days, followed by a drug holiday of the compound represented by Formula (I) and/or binimetinib, followed by administering to the patient each of the compound represented by Formula (I) and binimetinib for from 1 to 14 consecutive days. In some embodiments, the method comprises administering to the patient each of the compound represented by Formula (I) and binimetinib for from 1 to 7 consecutive days, followed by a drug holiday of the compound represented by Formula (I) and/or binimetinib, followed by administering to the patient each of the compound represented by Formula (I) and binimetinib for from 1 to 7 consecutive days.

In some embodiments, the method comprises administering to the patient each of the compound represented by Formula (I) and binimetinib for from 1 to 30 consecutive days, followed by a drug holiday of the compound represented by Formula (I) and/or binimetinib, followed by administering to the patient each of the compound represented by Formula (I) and binimetinib for from 1 to 7 consecutive days. In some embodiments, the method comprises administering to the patient each of the compound represented by Formula (I) and binimetinib for from 1 to 30 consecutive days, followed by a drug holiday of the compound represented by Formula (I) and/or binimetinib, followed by administering to the patient each of the compound represented by Formula (I) and binimetinib for from 1 to 14 consecutive days. In some embodiments, the method comprises administering to the patient each of the compound represented by Formula (I) and binimetinib for from 1 to 30 consecutive days, followed by a drug holiday of the compound represented by Formula (I) and/or binimetinib, followed by administering to the patient each of the compound represented by Formula (I) and binimetinib for from 1 to 21 consecutive days. In some embodiments, the method comprises administering to the patient each of the compound represented by Formula (I) and binimetinib for from 1 to 30 consecutive days, followed by a drug holiday of the compound represented by Formula (I) and/or binimetinib, followed by administering to the patient each of the compound represented by Formula (I) and binimetinib for from 1 to 28 consecutive days.

In some embodiments, the method comprises administering to the patient each of the compound represented by Formula (I) and binimetinib for from 1 to 21 consecutive days, followed by a drug holiday of the compound represented by Formula (I) and/or binimetinib, followed by administering to the patient each of the compound represented by Formula (I) and binimetinib for from 1 to 7 consecutive days. In some embodiments, the method comprises administering to the patient each of the compound represented by Formula (I) and binimetinib for from 1 to 21 consecutive days, followed by a drug holiday of the compound represented by Formula (I) and/or binimetinib, followed by administering to the patient each of the compound represented by Formula (I) and binimetinib for from 1 to 14 consecutive days. In some embodiments, the method comprises administering to the patient each of the compound represented by Formula (I) and binimetinib for from 1 to 21 consecutive days, followed by a drug holiday of the compound represented by Formula (I) and/or binimetinib, followed by administering to the patient each of the compound represented by Formula (I) and binimetinib for from 1 to 28 consecutive days. In some embodiments, the method comprises administering to the patient each of the compound represented by Formula (I) and binimetinib for from 1 to 21 consecutive days, followed by a drug holiday of the compound represented by Formula (I) and/or binimetinib, followed by administering to the patient each of the compound represented by Formula (I) and binimetinib for from 1 to 30 consecutive days.

In some embodiments, the method comprises administering to the patient each of the compound represented by Formula (I) and binimetinib for from 1 to 14 consecutive days, followed by a drug holiday of the compound represented by Formula (I) and/or binimetinib, followed by administering to the patient each of the compound represented by Formula (I) and binimetinib for from 1 to 7 consecutive days. In some embodiments, the method comprises administering to the patient each of the compound represented by Formula (I) and binimetinib for from 1 to 14 consecutive days, followed by a drug holiday of the compound represented by Formula (I) and/or binimetinib, followed by administering to the patient each of the compound represented by Formula (I) and binimetinib for from 1 to 21 consecutive days. In some embodiments, the method comprises administering to the patient each of the compound represented by Formula (I) and binimetinib for from 1 to 14 consecutive days, followed by a drug holiday of the compound represented by Formula (I) and/or binimetinib, followed by administering to the patient each of the compound represented by Formula (I) and binimetinib for from 1 to 28 consecutive days. In some embodiments, the method comprises administering to the patient each of the compound represented by Formula (I) and binimetinib for from 1 to 14 consecutive days, followed by a drug holiday of the compound represented by Formula (I) and/or binimetinib, followed by administering to the patient each of the compound represented by Formula (I) and binimetinib for from 1 to 30 consecutive days.

In some embodiments, the method comprises administering to the patient each of the compound represented by Formula (I) and binimetinib for from 1 to 7 consecutive days, followed by a drug holiday of the compound represented by Formula (I) and/or binimetinib, followed by administering to the patient each of the compound represented by Formula (I) and binimetinib for from 1 to 14 consecutive days. In some embodiments, the method comprises administering to the patient each of the compound represented by Formula (I) and binimetinib for from 1 to 7 consecutive days, followed by a drug holiday of the compound represented by Formula (I) and/or binimetinib, followed by administering to the patient each of the compound represented by Formula (I) and binimetinib for from 1 to 21 consecutive days. In some embodiments, the method comprises administering to the patient each of the compound represented by Formula (I) and binimetinib for from 1 to 7 consecutive days, followed by a drug holiday of the compound represented by Formula (I) and/or binimetinib, followed by administering to the patient each of the compound represented by Formula (I) and binimetinib for from 1 to 28 consecutive days. In some embodiments, the method comprises administering to the patient each of the compound represented by Formula (I) and binimetinib for from 1 to 7 consecutive days, followed by a drug holiday of the compound represented by Formula (I) and/or binimetinib, followed by administering to the patient each of the compound represented by Formula (I) and binimetinib for from 1 to 30 consecutive days.

In some embodiments, the method comprises administering to the patient each of the compound represented by Formula (I) and binimetinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or binimetinib, followed by administering to the patient each of the compound represented by Formula (I) and binimetinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and binimetinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or binimetinib, followed by administering to the patient each of the compound represented by Formula (I) and binimetinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or binimetinib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or binimetinib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or binimetinib is a period of 1-4 weeks. In some embodiments, the drug holiday of the compound represented by Formula (I) and/or binimetinib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of binimetinib in the patient. In some embodiments, the drug holiday is about 1-3 times the half-life of binimetinib in the patient. In some embodiments, the drug holiday is about 1-4 times the half-life of binimetinib in the patient. In some embodiments, the drug holiday is about 1-5 times the half-life of binimetinib in the patient. In some embodiments, the drug holiday is about 1-6 times the half-life of binimetinib in the patient. In some embodiments, the drug holiday is about 4-5 times the half-life of binimetinib in the patient. In some embodiments, the drug holiday is about 4 times the half-life of binimetinib in the patient. In some embodiments, the drug holiday is about 5 times the half-life of binimetinib in the patient.

Combinations with KRAS G12C Inhibitors

In another embodiment, described herein is a method of treating cancer in a patient in need thereof, comprising administering to the patient: (i) administering to the patient about 20 mg to about 1200 mg, daily, of a compound represented by Formula (I):

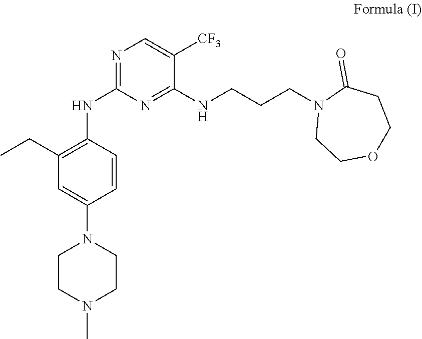 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of a KRAS G12C inhibitor. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the KRAS G12C inhibitor is sotorasib. In some embodiments, the method comprises administering to the patient about 960 mg, once daily, of sotorasib. In some embodiments, the method comprises administering to the patient about 100 mg to about 300 mg, once daily, of sotorasib. In some embodiments, the method comprises administering to the patient about 120 mg, once daily, of sotorasib. In some embodiments, the method comprises administering to the patient about 140 mg, once daily, of sotorasib. In some embodiments, the method comprises administering to the patient about 160 mg, once daily, of sotorasib. In some embodiments, the method comprises administering to the patient about 180 mg, once daily, of sotorasib. In some embodiments, the method comprises administering to the patient about 200 mg, once daily, of sotorasib. In some embodiments, the method comprises administering to the patient about 220 mg, once daily, of sotorasib. In some embodiments, the method comprises administering to the patient about 240 mg, once daily, of sotorasib. In some embodiments, the method comprises administering to the patient about 260 mg, once daily, of sotorasib. In some embodiments, the method comprises administering to the patient about 280 mg, once daily, of sotorasib. In some embodiments, the method comprises administering to the patient about 300 mg, once daily, of sotorasib. In some embodiments, the method comprises administering to the patient about 960 mg, twice daily, of sotorasib. In some embodiments, the method comprises administering to the patient about 100 mg to about 300 mg, twice daily, of sotorasib. In some embodiments, the method comprises administering to the patient about 120 mg, twice daily, of sotorasib. In some embodiments, the method comprises administering to the patient about 140 mg, twice daily, of sotorasib. In some embodiments, the method comprises administering to the patient about 160 mg, twice daily, of sotorasib. In some embodiments, the method comprises administering to the patient about 180 mg, twice daily, of sotorasib. In some embodiments, the method comprises administering to the patient about 200 mg, twice daily, of sotorasib. In some embodiments, the method comprises administering to the patient about 220 mg, twice daily, of sotorasib. In some embodiments, the method comprises administering to the patient about 240 mg, twice daily, of sotorasib. In some embodiments, the method comprises administering to the patient about 260 mg, twice daily, of sotorasib. In some embodiments, the method comprises administering to the patient about 280 mg, twice daily, of sotorasib. In some embodiments, the method comprises administering to the patient about 300 mg, twice daily, of sotorasib. In some embodiments, the KRAS G12C inhibitor is adagrasib. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In another embodiment, described herein is a method of treating cancer in a patient in need thereof, comprising administering to the patient: (i) administering to the patient about 20 mg to about 600 mg, once or twice daily, of a compound represented by Formula (I):

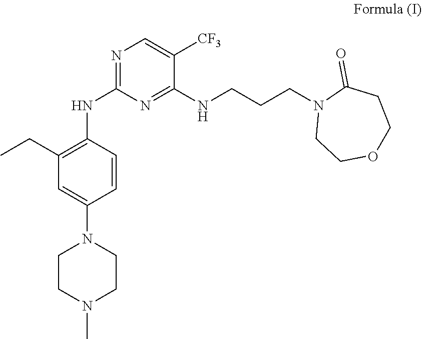 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of a KRAS G12C inhibitor. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the KRAS G12C inhibitor is sotorasib. In some embodiments, the method comprises administering to the patient about 960 mg, once daily, of sotorasib. In some embodiments, the method comprises administering to the patient about 100 mg to about 300 mg, once daily, of sotorasib. In some embodiments, the method comprises administering to the patient about 120 mg, once daily, of sotorasib. In some embodiments, the method comprises administering to the patient about 140 mg, once daily, of sotorasib. In some embodiments, the method comprises administering to the patient about 160 mg, once daily, of sotorasib. In some embodiments, the method comprises administering to the patient about 180 mg, once daily, of sotorasib. In some embodiments, the method comprises administering to the patient about 200 mg, once daily, of sotorasib. In some embodiments, the method comprises administering to the patient about 220 mg, once daily, of sotorasib. In some embodiments, the method comprises administering to the patient about 240 mg, once daily, of sotorasib. In some embodiments, the method comprises administering to the patient about 260 mg, once daily, of sotorasib. In some embodiments, the method comprises administering to the patient about 280 mg, once daily, of sotorasib. In some embodiments, the method comprises administering to the patient about 300 mg, once daily, of sotorasib. In some embodiments, the method comprises administering to the patient about 960 mg, twice daily, of sotorasib. In some embodiments, the method comprises administering to the patient about 100 mg to about 300 mg, twice daily, of sotorasib. In some embodiments, the method comprises administering to the patient about 120 mg, twice daily, of sotorasib. In some embodiments, the method comprises administering to the patient about 140 mg, twice daily, of sotorasib. In some embodiments, the method comprises administering to the patient about 160 mg, twice daily, of sotorasib. In some embodiments, the method comprises administering to the patient about 180 mg, twice daily, of sotorasib. In some embodiments, the method comprises administering to the patient about 200 mg, twice daily, of sotorasib. In some embodiments, the method comprises administering to the patient about 220 mg, twice daily, of sotorasib. In some embodiments, the method comprises administering to the patient about 240 mg, twice daily, of sotorasib. In some embodiments, the method comprises administering to the patient about 260 mg, twice daily, of sotorasib. In some embodiments, the method comprises administering to the patient about 280 mg, twice daily, of sotorasib. In some embodiments, the method comprises administering to the patient about 300 mg, twice daily, of sotorasib. In some embodiments, the KRAS G12C inhibitor is adagrasib. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In some embodiments, the method comprises administering to the patient each of the compound represented by Formula (I) and the KRAS G12C inhibitor for from 1 to 30 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 30 consecutive days. In some embodiments, the method comprises administering to the patient each of the compound represented by Formula (I) and the KRAS G12C inhibitor for from 1 to 28 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 28 consecutive days. In some embodiments, the method comprises administering to the patient each of the compound represented by Formula (I) and the KRAS G12C inhibitor for from 1 to 21 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 21 consecutive days. In some embodiments, the method comprises administering to the patient each of the compound represented by Formula (I) and the KRAS G12C inhibitor for from 1 to 14 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 14 consecutive days. In some embodiments, the method comprises administering to the patient each of the compound represented by Formula (I) and the KRAS G12C inhibitor for from 1 to 7 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 7 consecutive days.

In some embodiments, the method comprises administering to the patient each of the compound represented by Formula (I) and the KRAS G12C inhibitor for from 1 to 30 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 7 consecutive days. In some embodiments, the method comprises administering to the patient each of the compound represented by Formula (I) and the KRAS G12C inhibitor for from 1 to 30 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 14 consecutive days. In some embodiments, the method comprises administering to the patient each of the compound represented by Formula (I) and the KRAS G12C inhibitor for from 1 to 30 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 21 consecutive days. In some embodiments, the method comprises administering to the patient each of the compound represented by Formula (I) and the KRAS G12C inhibitor for from 1 to 30 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 28 consecutive days.

In some embodiments, the method comprises administering to the patient each of the compound represented by Formula (I) and the KRAS G12C inhibitor for from 1 to 21 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 7 consecutive days. In some embodiments, the method comprises administering to the patient each of the compound represented by Formula (I) and the KRAS G12C inhibitor for from 1 to 21 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 14 consecutive days. In some embodiments, the method comprises administering to the patient each of the compound represented by Formula (I) and the KRAS G12C inhibitor for from 1 to 21 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 28 consecutive days. In some embodiments, the method comprises administering to the patient each of the compound represented by Formula (I) and the KRAS G12C inhibitor for from 1 to 21 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 30 consecutive days.

In some embodiments, the method comprises administering to the patient each of the compound represented by Formula (I) and the KRAS G12C inhibitor for from 1 to 14 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 7 consecutive days. In some embodiments, the method comprises administering to the patient each of the compound represented by Formula (I) and the KRAS G12C inhibitor for from 1 to 14 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 21 consecutive days. In some embodiments, the method comprises administering to the patient each of the compound represented by Formula (I) and the KRAS G12C inhibitor for from 1 to 14 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 28 consecutive days. In some embodiments, the method comprises administering to the patient each of the compound represented by Formula (I) and the KRAS G12C inhibitor for from 1 to 14 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 30 consecutive days.

In some embodiments, the method comprises administering to the patient each of the compound represented by Formula (I) and the KRAS G12C inhibitor for from 1 to 7 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 14 consecutive days. In some embodiments, the method comprises administering to the patient each of the compound represented by Formula (I) and the KRAS G12C inhibitor for from 1 to 7 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 21 consecutive days. In some embodiments, the method comprises administering to the patient each of the compound represented by Formula (I) and the KRAS G12C inhibitor for from 1 to 7 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 28 consecutive days. In some embodiments, the method comprises administering to the patient each of the compound represented by Formula (I) and the KRAS G12C inhibitor for from 1 to 7 consecutive days, followed by a drug holiday of the compound represented by Formula (I) and/or the KRAS G12C inhibitor, followed by administering to the patient each of the compound represented by Formula (I) and the KRAS G12C inhibitor for from 1 to 30 consecutive days.

In some embodiments, the method comprises administering to the patient each of the compound represented by Formula (I) and the KRAS G12C inhibitor for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or the KRAS G12C inhibitor, followed by administering to the patient each of the compound represented by Formula (I) and the KRAS G12C inhibitor for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and the KRAS G12C inhibitor for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or the KRAS G12C inhibitor, followed by administering to the patient each of the compound represented by Formula (I) and the KRAS G12C inhibitor for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or the KRAS G12C inhibitor is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or the KRAS G12C inhibitor is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or the KRAS G12C inhibitor is a period of 1-4 weeks. In some embodiments, the drug holiday of the compound represented by Formula (I) and/or the KRAS G12C inhibitor is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of the KRAS G12C inhibitor in the patient. In some embodiments, the drug holiday is about 1-3 times the half-life of the KRAS G12C inhibitor in the patient. In some embodiments, the drug holiday is about 1-4 times the half-life of the KRAS G12C inhibitor in the patient. In some embodiments, the drug holiday is about 1-5 times the half-life of the KRAS G12C inhibitor in the patient. In some embodiments, the drug holiday is about 1-6 times the half-life of the KRAS G12C inhibitor in the patient. In some embodiments, the drug holiday is about 4-5 times the half-life of the KRAS G12C inhibitor in the patient. In some embodiments, the drug holiday is about 4 times the half-life of the KRAS G12C inhibitor in the patient. In some embodiments, the drug holiday is about 5 times the half-life of the KRAS G12C inhibitor in the patient.

Combinations with Sotorasib

In another embodiment, described herein is a method of treating cancer in a patient in need thereof, comprising administering to the patient: (i) orally administering to the patient about 20 mg to about 1200 mg, daily, of a compound represented by Formula (I):

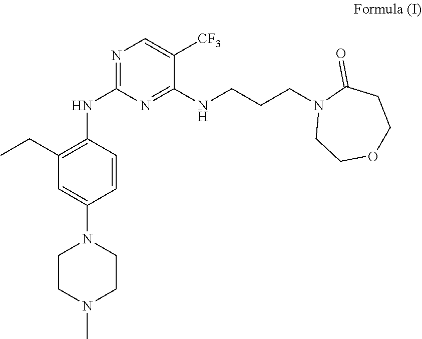 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of sotorasib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 960 mg, once daily, of sotorasib. In some embodiments, the method comprises administering to the patient about 100 mg to about 300 mg, once daily, of sotorasib. In some embodiments, the method comprises administering to the patient about 120 mg, once daily, of sotorasib. In some embodiments, the method comprises administering to the patient about 140 mg, once daily, of sotorasib. In some embodiments, the method comprises administering to the patient about 160 mg, once daily, of sotorasib. In some embodiments, the method comprises administering to the patient about 180 mg, once daily, of sotorasib. In some embodiments, the method comprises administering to the patient about 200 mg, once daily, of sotorasib. In some embodiments, the method comprises administering to the patient about 220 mg, once daily, of sotorasib. In some embodiments, the method comprises administering to the patient about 240 mg, once daily, of sotorasib. In some embodiments, the method comprises administering to the patient about 260 mg, once daily, of sotorasib. In some embodiments, the method comprises administering to the patient about 280 mg, once daily, of sotorasib. In some embodiments, the method comprises administering to the patient about 300 mg, once daily, of sotorasib. In some embodiments, the method comprises administering to the patient about 960 mg, twice daily, of sotorasib. In some embodiments, the method comprises administering to the patient about 100 mg to about 300 mg, twice daily, of sotorasib. In some embodiments, the method comprises administering to the patient about 120 mg, twice daily, of sotorasib. In some embodiments, the method comprises administering to the patient about 140 mg, twice daily, of sotorasib. In some embodiments, the method comprises administering to the patient about 160 mg, twice daily, of sotorasib. In some embodiments, the method comprises administering to the patient about 180 mg, twice daily, of sotorasib. In some embodiments, the method comprises administering to the patient about 200 mg, twice daily, of sotorasib. In some embodiments, the method comprises administering to the patient about 220 mg, twice daily, of sotorasib. In some embodiments, the method comprises administering to the patient about 240 mg, twice daily, of sotorasib. In some embodiments, the method comprises administering to the patient about 260 mg, twice daily, of sotorasib. In some embodiments, the method comprises administering to the patient about 280 mg, twice daily, of sotorasib. In some embodiments, the method comprises administering to the patient about 300 mg, twice daily, of sotorasib. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In another embodiment, described herein is a method of treating cancer in a patient in need thereof, comprising administering to the patient: (i) orally administering to the patient about 20 mg to about 600 mg, once or twice daily, of a compound represented by Formula (I):

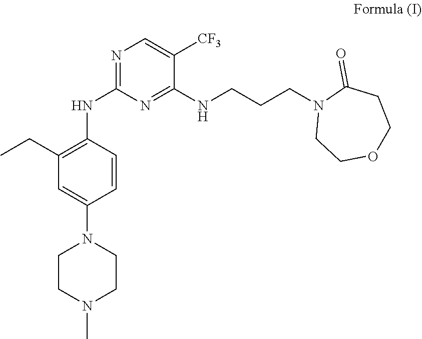 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of sotorasib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 960 mg, once daily, of sotorasib. In some embodiments, the method comprises administering to the patient about 100 mg to about 300 mg, once daily, of sotorasib. In some embodiments, the method comprises administering to the patient about 120 mg, once daily, of sotorasib. In some embodiments, the method comprises administering to the patient about 140 mg, once daily, of sotorasib. In some embodiments, the method comprises administering to the patient about 160 mg, once daily, of sotorasib. In some embodiments, the method comprises administering to the patient about 180 mg, once daily, of sotorasib. In some embodiments, the method comprises administering to the patient about 200 mg, once daily, of sotorasib. In some embodiments, the method comprises administering to the patient about 220 mg, once daily, of sotorasib. In some embodiments, the method comprises administering to the patient about 240 mg, once daily, of sotorasib. In some embodiments, the method comprises administering to the patient about 260 mg, once daily, of sotorasib. In some embodiments, the method comprises administering to the patient about 280 mg, once daily, of sotorasib. In some embodiments, the method comprises administering to the patient about 300 mg, once daily, of sotorasib. In some embodiments, the method comprises administering to the patient about 960 mg, twice daily, of sotorasib. In some embodiments, the method comprises administering to the patient about 100 mg to about 300 mg, twice daily, of sotorasib. In some embodiments, the method comprises administering to the patient about 120 mg, twice daily, of sotorasib. In some embodiments, the method comprises administering to the patient about 140 mg, twice daily, of sotorasib. In some embodiments, the method comprises administering to the patient about 160 mg, twice daily, of sotorasib. In some embodiments, the method comprises administering to the patient about 180 mg, twice daily, of sotorasib. In some embodiments, the method comprises administering to the patient about 200 mg, twice daily, of sotorasib. In some embodiments, the method comprises administering to the patient about 220 mg, twice daily, of sotorasib. In some embodiments, the method comprises administering to the patient about 240 mg, twice daily, of sotorasib. In some embodiments, the method comprises administering to the patient about 260 mg, twice daily, of sotorasib. In some embodiments, the method comprises administering to the patient about 280 mg, twice daily, of sotorasib. In some embodiments, the method comprises administering to the patient about 300 mg, twice daily, of sotorasib. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In some embodiments, the method comprises administering to the patient each of the compound represented by Formula (I) and sotorasib for from 1 to 30 consecutive days, followed by a drug holiday of the compound represented by Formula (I) and/or sotorasib, followed by administering to the patient each of the compound represented by Formula (I) and sotorasib for from 1 to 30 consecutive days. In some embodiments, the method comprises administering to the patient each of the compound represented by Formula (I) and sotorasib for from 1 to 28 consecutive days, followed by a drug holiday of the compound represented by Formula (I) and/or sotorasib, followed by administering to the patient each of the compound represented by Formula (I) and sotorasib for from 1 to 28 consecutive days. In some embodiments, the method comprises administering to the patient each of the compound represented by Formula (I) and sotorasib for from 1 to 21 consecutive days, followed by a drug holiday of the compound represented by Formula (I) and/or sotorasib, followed by administering to the patient each of the compound represented by Formula (I) and sotorasib for from 1 to 21 consecutive days. In some embodiments, the method comprises administering to the patient each of the compound represented by Formula (I) and sotorasib for from 1 to 14 consecutive days, followed by a drug holiday of the compound represented by Formula (I) and/or sotorasib, followed by administering to the patient each of the compound represented by Formula (I) and sotorasib for from 1 to 14 consecutive days. In some embodiments, the method comprises administering to the patient each of the compound represented by Formula (I) and sotorasib for from 1 to 7 consecutive days, followed by a drug holiday of the compound represented by Formula (I) and/or sotorasib, followed by administering to the patient each of the compound represented by Formula (I) and sotorasib for from 1 to 7 consecutive days.

In some embodiments, the method comprises administering to the patient each of the compound represented by Formula (I) and sotorasib for from 1 to 30 consecutive days, followed by a drug holiday of the compound represented by Formula (I) and/or sotorasib, followed by administering to the patient each of the compound represented by Formula (I) and sotorasib for from 1 to 7 consecutive days. In some embodiments, the method comprises administering to the patient each of the compound represented by Formula (I) and sotorasib for from 1 to 30 consecutive days, followed by a drug holiday of the compound represented by Formula (I) and/or sotorasib, followed by administering to the patient each of the compound represented by Formula (I) and sotorasib for from 1 to 14 consecutive days. In some embodiments, the method comprises administering to the patient each of the compound represented by Formula (I) and sotorasib for from 1 to 30 consecutive days, followed by a drug holiday of the compound represented by Formula (I) and/or sotorasib, followed by administering to the patient each of the compound represented by Formula (I) and sotorasib for from 1 to 21 consecutive days. In some embodiments, the method comprises administering to the patient each of the compound represented by Formula (I) and sotorasib for from 1 to 30 consecutive days, followed by a drug holiday of the compound represented by Formula (I) and/or sotorasib, followed by administering to the patient each of the compound represented by Formula (I) and sotorasib for from 1 to 28 consecutive days.

In some embodiments, the method comprises administering to the patient each of the compound represented by Formula (I) and sotorasib for from 1 to 21 consecutive days, followed by a drug holiday of the compound represented by Formula (I) and/or sotorasib, followed by administering to the patient each of the compound represented by Formula (I) and sotorasib for from 1 to 7 consecutive days. In some embodiments, the method comprises administering to the patient each of the compound represented by Formula (I) and sotorasib for from 1 to 21 consecutive days, followed by a drug holiday of the compound represented by Formula (I) and/or sotorasib, followed by administering to the patient each of the compound represented by Formula (I) and sotorasib for from 1 to 14 consecutive days. In some embodiments, the method comprises administering to the patient each of the compound represented by Formula (I) and sotorasib for from 1 to 21 consecutive days, followed by a drug holiday of the compound represented by Formula (I) and/or sotorasib, followed by administering to the patient each of the compound represented by Formula (I) and sotorasib for from 1 to 28 consecutive days. In some embodiments, the method comprises administering to the patient each of the compound represented by Formula (I) and sotorasib for from 1 to 21 consecutive days, followed by a drug holiday of the compound represented by Formula (I) and/or sotorasib, followed by administering to the patient each of the compound represented by Formula (I) and sotorasib for from 1 to 30 consecutive days.

In some embodiments, the method comprises administering to the patient each of the compound represented by Formula (I) and sotorasib for from 1 to 14 consecutive days, followed by a drug holiday of the compound represented by Formula (I) and/or sotorasib, followed by administering to the patient each of the compound represented by Formula (I) and sotorasib for from 1 to 7 consecutive days. In some embodiments, the method comprises administering to the patient each of the compound represented by Formula (I) and sotorasib for from 1 to 14 consecutive days, followed by a drug holiday of the compound represented by Formula (I) and/or sotorasib, followed by administering to the patient each of the compound represented by Formula (I) and sotorasib for from 1 to 21 consecutive days. In some embodiments, the method comprises administering to the patient each of the compound represented by Formula (I) and sotorasib for from 1 to 14 consecutive days, followed by a drug holiday of the compound represented by Formula (I) and/or sotorasib, followed by administering to the patient each of the compound represented by Formula (I) and sotorasib for from 1 to 28 consecutive days. In some embodiments, the method comprises administering to the patient each of the compound represented by Formula (I) and sotorasib for from 1 to 14 consecutive days, followed by a drug holiday of the compound represented by Formula (I) and/or sotorasib, followed by administering to the patient each of the compound represented by Formula (I) and sotorasib for from 1 to 30 consecutive days.

In some embodiments, the method comprises administering to the patient each of the compound represented by Formula (I) and sotorasib for from 1 to 7 consecutive days, followed by a drug holiday of the compound represented by Formula (I) and/or sotorasib, followed by administering to the patient each of the compound represented by Formula (I) and sotorasib for from 1 to 14 consecutive days. In some embodiments, the method comprises administering to the patient each of the compound represented by Formula (I) and sotorasib for from 1 to 7 consecutive days, followed by a drug holiday of the compound represented by Formula (I) and/or sotorasib, followed by administering to the patient each of the compound represented by Formula (I) and sotorasib for from 1 to 21 consecutive days. In some embodiments, the method comprises administering to the patient each of the compound represented by Formula (I) and sotorasib for from 1 to 7 consecutive days, followed by a drug holiday of the compound represented by Formula (I) and/or sotorasib, followed by administering to the patient each of the compound represented by Formula (I) and sotorasib for from 1 to 28 consecutive days. In some embodiments, the method comprises administering to the patient each of the compound represented by Formula (I) and sotorasib for from 1 to 7 consecutive days, followed by a drug holiday of the compound represented by Formula (I) and/or sotorasib, followed by administering to the patient each of the compound represented by Formula (I) and sotorasib for from 1 to 30 consecutive days.

In some embodiments, the method comprises administering to the patient each of the compound represented by Formula (I) and sotorasib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or sotorasib, followed by administering to the patient each of the compound represented by Formula (I) and sotorasib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and sotorasib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or sotorasib, followed by administering to the patient each of the compound represented by Formula (I) and sotorasib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or sotorasib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or sotorasib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or sotorasib is a period of 1-4 weeks. In some embodiments, the drug holiday of the compound represented by Formula (I) and/or sotorasib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of sotorasib in the patient. In some embodiments, the drug holiday is about 1-3 times the half-life of sotorasib in the patient. In some embodiments, the drug holiday is about 1-4 times the half-life of sotorasib in the patient. In some embodiments, the drug holiday is about 1-5 times the half-life of sotorasib in the patient. In some embodiments, the drug holiday is about 1-6 times the half-life of sotorasib in the patient. In some embodiments, the drug holiday is about 4-5 times the half-life of sotorasib in the patient. In some embodiments, the drug holiday is about 4 times the half-life of sotorasib in the patient. In some embodiments, the drug holiday is about 5 times the half-life of sotorasib in the patient.

Combinations with Adagrasib

In another embodiment, described herein is a method of treating cancer in a patient in need thereof, comprising administering to the patient: (i) orally administering to the patient about 20 mg to about 1200 mg, daily, of a compound represented by Formula (I):

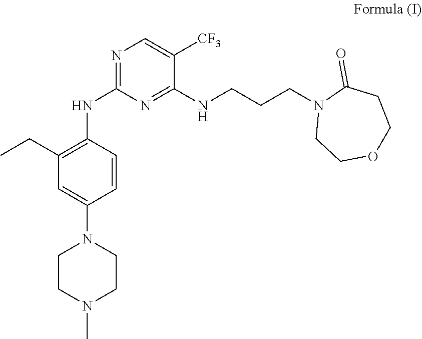 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of adagrasib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In another embodiment, described herein is a method of treating cancer in a patient in need thereof, comprising administering to the patient: (i) orally administering to the patient about 20 mg to about 600 mg, once or twice daily, of a compound represented by Formula (I):

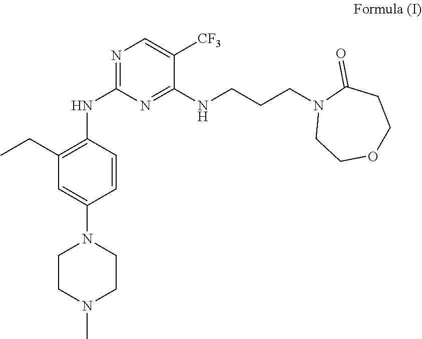 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of adagrasib. In some embodiments, the method comprises orally administering the compound to the patient. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the cancer is non-small cell lung cancer. In some embodiments, the non-small cell lung cancer has a KRAS G12C mutation. In some embodiments, the non-small cell lung cancer is a KRAS G12C-mutated locally advanced or metastatic non-small cell lung cancer. In some embodiments, the cancer is colorectal cancer. In some embodiments, the cancer is pancreatic cancer. In some embodiments, the colorectal cancer is a KRAS G12C-mutated colorectal cancer. In some embodiments, the pancreatic cancer is a KRAS G12C-mutated pancreatic cancer. In some embodiments, the pancreatic cancer is selected from the group consisting of a locally advanced pancreatic cancer, an unresectable pancreatic cancer, and a metastatic pancreatic cancer.

In some embodiments, the method comprises administering to the patient each of the compound represented by Formula (I) and adagrasib for from 1 to 30 consecutive days, followed by a drug holiday of the compound represented by Formula (I) and/or adagrasib, followed by administering to the patient each of the compound represented by Formula (I) and adagrasib for from 1 to 30 consecutive days. In some embodiments, the method comprises administering to the patient each of the compound represented by Formula (I) and adagrasib for from 1 to 28 consecutive days, followed by a drug holiday of the compound represented by Formula (I) and/or adagrasib, followed by administering to the patient each of the compound represented by Formula (I) and adagrasib for from 1 to 28 consecutive days. In some embodiments, the method comprises administering to the patient each of the compound represented by Formula (I) and adagrasib for from 1 to 21 consecutive days, followed by a drug holiday of the compound represented by Formula (I) and/or adagrasib, followed by administering to the patient each of the compound represented by Formula (I) and adagrasib for from 1 to 21 consecutive days. In some embodiments, the method comprises administering to the patient each of the compound represented by Formula (I) and adagrasib for from 1 to 14 consecutive days, followed by a drug holiday of the compound represented by Formula (I) and/or adagrasib, followed by administering to the patient each of the compound represented by Formula (I) and adagrasib for from 1 to 14 consecutive days. In some embodiments, the method comprises administering to the patient each of the compound represented by Formula (I) and adagrasib for from 1 to 7 consecutive days, followed by a drug holiday of the compound represented by Formula (I) and/or adagrasib, followed by administering to the patient each of the compound represented by Formula (I) and adagrasib for from 1 to 7 consecutive days.

In some embodiments, the method comprises administering to the patient each of the compound represented by Formula (I) and adagrasib for from 1 to 30 consecutive days, followed by a drug holiday of the compound represented by Formula (I) and/or adagrasib, followed by administering to the patient each of the compound represented by Formula (I) and adagrasib for from 1 to 7 consecutive days. In some embodiments, the method comprises administering to the patient each of the compound represented by Formula (I) and adagrasib for from 1 to 30 consecutive days, followed by a drug holiday of the compound represented by Formula (I) and/or adagrasib, followed by administering to the patient each of the compound represented by Formula (I) and adagrasib for from 1 to 14 consecutive days. In some embodiments, the method comprises administering to the patient each of the compound represented by Formula (I) and adagrasib for from 1 to 30 consecutive days, followed by a drug holiday of the compound represented by Formula (I) and/or adagrasib, followed by administering to the patient each of the compound represented by Formula (I) and adagrasib for from 1 to 21 consecutive days. In some embodiments, the method comprises administering to the patient each of the compound represented by Formula (I) and adagrasib for from 1 to 30 consecutive days, followed by a drug holiday of the compound represented by Formula (I) and/or adagrasib, followed by administering to the patient each of the compound represented by Formula (I) and adagrasib for from 1 to 28 consecutive days.

In some embodiments, the method comprises administering to the patient each of the compound represented by Formula (I) and adagrasib for from 1 to 21 consecutive days, followed by a drug holiday of the compound represented by Formula (I) and/or adagrasib, followed by administering to the patient each of the compound represented by Formula (I) and adagrasib for from 1 to 7 consecutive days. In some embodiments, the method comprises administering to the patient each of the compound represented by Formula (I) and adagrasib for from 1 to 21 consecutive days, followed by a drug holiday of the compound represented by Formula (I) and/or adagrasib, followed by administering to the patient each of the compound represented by Formula (I) and adagrasib for from 1 to 14 consecutive days. In some embodiments, the method comprises administering to the patient each of the compound represented by Formula (I) and adagrasib for from 1 to 21 consecutive days, followed by a drug holiday of the compound represented by Formula (I) and/or adagrasib, followed by administering to the patient each of the compound represented by Formula (I) and adagrasib for from 1 to 28 consecutive days. In some embodiments, the method comprises administering to the patient each of the compound represented by Formula (I) and adagrasib for from 1 to 21 consecutive days, followed by a drug holiday of the compound represented by Formula (I) and/or adagrasib, followed by administering to the patient each of the compound represented by Formula (I) and adagrasib for from 1 to 30 consecutive days.

In some embodiments, the method comprises administering to the patient each of the compound represented by Formula (I) and adagrasib for from 1 to 14 consecutive days, followed by a drug holiday of the compound represented by Formula (I) and/or adagrasib, followed by administering to the patient each of the compound represented by Formula (I) and adagrasib for from 1 to 7 consecutive days. In some embodiments, the method comprises administering to the patient each of the compound represented by Formula (I) and adagrasib for from 1 to 14 consecutive days, followed by a drug holiday of the compound represented by Formula (I) and/or adagrasib, followed by administering to the patient each of the compound represented by Formula (I) and adagrasib for from 1 to 21 consecutive days. In some embodiments, the method comprises administering to the patient each of the compound represented by Formula (I) and adagrasib for from 1 to 14 consecutive days, followed by a drug holiday of the compound represented by Formula (I) and/or adagrasib, followed by administering to the patient each of the compound represented by Formula (I) and adagrasib for from 1 to 28 consecutive days. In some embodiments, the method comprises administering to the patient each of the compound represented by Formula (I) and adagrasib for from 1 to 14 consecutive days, followed by a drug holiday of the compound represented by Formula (I) and/or adagrasib, followed by administering to the patient each of the compound represented by Formula (I) and adagrasib for from 1 to 30 consecutive days.

In some embodiments, the method comprises administering to the patient each of the compound represented by Formula (I) and adagrasib for from 1 to 7 consecutive days, followed by a drug holiday of the compound represented by Formula (I) and/or adagrasib, followed by administering to the patient each of the compound represented by Formula (I) and adagrasib for from 1 to 14 consecutive days. In some embodiments, the method comprises administering to the patient each of the compound represented by Formula (I) and adagrasib for from 1 to 7 consecutive days, followed by a drug holiday of the compound represented by Formula (I) and/or adagrasib, followed by administering to the patient each of the compound represented by Formula (I) and adagrasib for from 1 to 21 consecutive days. In some embodiments, the method comprises administering to the patient each of the compound represented by Formula (I) and adagrasib for from 1 to 7 consecutive days, followed by a drug holiday of the compound represented by Formula (I) and/or adagrasib, followed by administering to the patient each of the compound represented by Formula (I) and adagrasib for from 1 to 28 consecutive days. In some embodiments, the method comprises administering to the patient each of the compound represented by Formula (I) and adagrasib for from 1 to 7 consecutive days, followed by a drug holiday of the compound represented by Formula (I) and/or adagrasib, followed by administering to the patient each of the compound represented by Formula (I) and adagrasib for from 1 to 30 consecutive days.

In some embodiments, the method comprises administering to the patient each of the compound represented by Formula (I) and adagrasib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or adagrasib, followed by administering to the patient each of the compound represented by Formula (I) and adagrasib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and adagrasib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or adagrasib, followed by administering to the patient each of the compound represented by Formula (I) and adagrasib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or adagrasib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or adagrasib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or adagrasib is a period of 1-4 weeks. In some embodiments, the drug holiday of the compound represented by Formula (I) and/or adagrasib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of adagrasib in the patient. In some embodiments, the drug holiday is about 1-3 times the half-life of adagrasib in the patient. In some embodiments, the drug holiday is about 1-4 times the half-life of adagrasib in the patient. In some embodiments, the drug holiday is about 1-5 times the half-life of adagrasib in the patient. In some embodiments, the drug holiday is about 1-6 times the half-life of adagrasib in the patient. In some embodiments, the drug holiday is about 4-5 times the half-life of adagrasib in the patient. In some embodiments, the drug holiday is about 4 times the half-life of adagrasib in the patient. In some embodiments, the drug holiday is about 5 times the half-life of adagrasib in the patient.

Combinations with Encorafenib

In another embodiment, described herein is a method of treating colorectal cancer in a patient in need thereof, comprising: (i) administering to the patient a therapeutically effective amount of a compound represented by Formula (I):

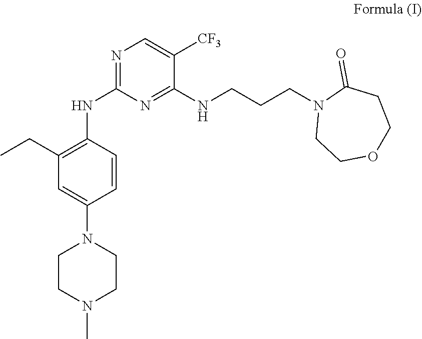 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of encorafenib. In some embodiments, the method comprises orally administering the compound to the patient.

In some embodiments, the method comprises administering to the patient each of the compound represented by Formula (I) and encorafenib for from 1 to 30 consecutive days, followed by a drug holiday of the compound represented by Formula (I) and/or encorafenib, followed by administering to the patient each of the compound represented by Formula (I) and encorafenib for from 1 to 30 consecutive days. In some embodiments, the method comprises administering to the patient each of the compound represented by Formula (I) and encorafenib for from 1 to 28 consecutive days, followed by a drug holiday of the compound represented by Formula (I) and/or encorafenib, followed by administering to the patient each of the compound represented by Formula (I) and encorafenib for from 1 to 28 consecutive days. In some embodiments, the method comprises administering to the patient each of the compound represented by Formula (I) and encorafenib for from 1 to 21 consecutive days, followed by a drug holiday of the compound represented by Formula (I) and/or encorafenib, followed by administering to the patient each of the compound represented by Formula (I) and encorafenib for from 1 to 21 consecutive days. In some embodiments, the method comprises administering to the patient each of the compound represented by Formula (I) and encorafenib for from 1 to 14 consecutive days, followed by a drug holiday of the compound represented by Formula (I) and/or encorafenib, followed by administering to the patient each of the compound represented by Formula (I) and encorafenib for from 1 to 14 consecutive days. In some embodiments, the method comprises administering to the patient each of the compound represented by Formula (I) and encorafenib for from 1 to 7 consecutive days, followed by a drug holiday of the compound represented by Formula (I) and/or encorafenib, followed by administering to the patient each of the compound represented by Formula (I) and encorafenib for from 1 to 7 consecutive days.

In some embodiments, the method comprises administering to the patient each of the compound represented by Formula (I) and encorafenib for from 1 to 30 consecutive days, followed by a drug holiday of the compound represented by Formula (I) and/or encorafenib, followed by administering to the patient each of the compound represented by Formula (I) and encorafenib for from 1 to 7 consecutive days. In some embodiments, the method comprises administering to the patient each of the compound represented by Formula (I) and encorafenib for from 1 to 30 consecutive days, followed by a drug holiday of the compound represented by Formula (I) and/or encorafenib, followed by administering to the patient each of the compound represented by Formula (I) and encorafenib for from 1 to 14 consecutive days. In some embodiments, the method comprises administering to the patient each of the compound represented by Formula (I) and encorafenib for from 1 to 30 consecutive days, followed by a drug holiday of the compound represented by Formula (I) and/or encorafenib, followed by administering to the patient each of the compound represented by Formula (I) and encorafenib for from 1 to 21 consecutive days. In some embodiments, the method comprises administering to the patient each of the compound represented by Formula (I) and encorafenib for from 1 to 30 consecutive days, followed by a drug holiday of the compound represented by Formula (I) and/or encorafenib, followed by administering to the patient each of the compound represented by Formula (I) and encorafenib for from 1 to 28 consecutive days.

In some embodiments, the method comprises administering to the patient each of the compound represented by Formula (I) and encorafenib for from 1 to 21 consecutive days, followed by a drug holiday of the compound represented by Formula (I) and/or encorafenib, followed by administering to the patient each of the compound represented by Formula (I) and encorafenib for from 1 to 7 consecutive days. In some embodiments, the method comprises administering to the patient each of the compound represented by Formula (I) and encorafenib for from 1 to 21 consecutive days, followed by a drug holiday of the compound represented by Formula (I) and/or encorafenib, followed by administering to the patient each of the compound represented by Formula (I) and encorafenib for from 1 to 14 consecutive days. In some embodiments, the method comprises administering to the patient each of the compound represented by Formula (I) and encorafenib for from 1 to 21 consecutive days, followed by a drug holiday of the compound represented by Formula (I) and/or encorafenib, followed by administering to the patient each of the compound represented by Formula (I) and encorafenib for from 1 to 28 consecutive days. In some embodiments, the method comprises administering to the patient each of the compound represented by Formula (I) and encorafenib for from 1 to 21 consecutive days, followed by a drug holiday of the compound represented by Formula (I) and/or encorafenib, followed by administering to the patient each of the compound represented by Formula (I) and encorafenib for from 1 to 30 consecutive days.

In some embodiments, the method comprises administering to the patient each of the compound represented by Formula (I) and encorafenib for from 1 to 14 consecutive days, followed by a drug holiday of the compound represented by Formula (I) and/or encorafenib, followed by administering to the patient each of the compound represented by Formula (I) and encorafenib for from 1 to 7 consecutive days. In some embodiments, the method comprises administering to the patient each of the compound represented by Formula (I) and encorafenib for from 1 to 14 consecutive days, followed by a drug holiday of the compound represented by Formula (I) and/or encorafenib, followed by administering to the patient each of the compound represented by Formula (I) and encorafenib for from 1 to 21 consecutive days. In some embodiments, the method comprises administering to the patient each of the compound represented by Formula (I) and encorafenib for from 1 to 14 consecutive days, followed by a drug holiday of the compound represented by Formula (I) and/or encorafenib, followed by administering to the patient each of the compound represented by Formula (I) and encorafenib for from 1 to 28 consecutive days. In some embodiments, the method comprises administering to the patient each of the compound represented by Formula (I) and encorafenib for from 1 to 14 consecutive days, followed by a drug holiday of the compound represented by Formula (I) and/or encorafenib, followed by administering to the patient each of the compound represented by Formula (I) and encorafenib for from 1 to 30 consecutive days.

In some embodiments, the method comprises administering to the patient each of the compound represented by Formula (I) and encorafenib for from 1 to 7 consecutive days, followed by a drug holiday of the compound represented by Formula (I) and/or encorafenib, followed by administering to the patient each of the compound represented by Formula (I) and encorafenib for from 1 to 14 consecutive days. In some embodiments, the method comprises administering to the patient each of the compound represented by Formula (I) and encorafenib for from 1 to 7 consecutive days, followed by a drug holiday of the compound represented by Formula (I) and/or encorafenib, followed by administering to the patient each of the compound represented by Formula (I) and encorafenib for from 1 to 21 consecutive days. In some embodiments, the method comprises administering to the patient each of the compound represented by Formula (I) and encorafenib for from 1 to 7 consecutive days, followed by a drug holiday of the compound represented by Formula (I) and/or encorafenib, followed by administering to the patient each of the compound represented by Formula (I) and encorafenib for from 1 to 28 consecutive days. In some embodiments, the method comprises administering to the patient each of the compound represented by Formula (I) and encorafenib for from 1 to 7 consecutive days, followed by a drug holiday of the compound represented by Formula (I) and/or encorafenib, followed by administering to the patient each of the compound represented by Formula (I) and encorafenib for from 1 to 30 consecutive days.

In some embodiments, the method comprises administering to the patient each of the compound represented by Formula (I) and encorafenib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or encorafenib, followed by administering to the patient each of the compound represented by Formula (I) and encorafenib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and encorafenib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or encorafenib, followed by administering to the patient each of the compound represented by Formula (I) and encorafenib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or encorafenib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or encorafenib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or encorafenib is a period of 1-4 weeks. In some embodiments, the drug holiday of the compound represented by Formula (I) and/or encorafenib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of encorafenib in the patient. In some embodiments, the drug holiday is about 1-3 times the half-life of encorafenib in the patient. In some embodiments, the drug holiday is about 1-4 times the half-life of encorafenib in the patient. In some embodiments, the drug holiday is about 1-5 times the half-life of encorafenib in the patient. In some embodiments, the drug holiday is about 1-6 times the half-life of encorafenib in the patient. In some embodiments, the drug holiday is about 4-5 times the half-life of encorafenib in the patient. In some embodiments, the drug holiday is about 4 times the half-life of encorafenib in the patient. In some embodiments, the drug holiday is about 5 times the half-life of encorafenib in the patient.

Combinations with Encorafenib and Cetuximab

In another embodiment, described herein is a method of treating colorectal cancer in a patient in need thereof, comprising: (i) administering to the patient a therapeutically effective amount of a compound represented by Formula (I):

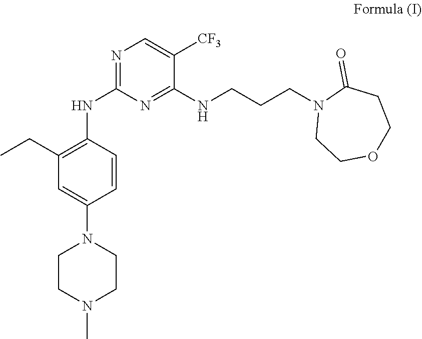 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) administering to the patient a therapeutically effective amount of encorafenib, and (iii) administering to the patient a therapeutically effective amount of cetuximab. In some embodiments, the method comprises orally administering the compound to the patient. In some embodiments, the method comprises administering to the patient about 20 mg to about 1200 mg, daily, of the compound or pharmaceutically acceptable salt thereof. In some embodiments, the method comprises administering to the patient about 20 mg to about 600 mg, once or twice daily, of the compound or pharmaceutically acceptable salt thereof. In some embodiments, the method comprises administering to the patient about 20 mg to about 400 mg, twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 300 mg to about 500 mg, once daily, of encorafenib. In some embodiments, the method comprises administering to the patient about 300 mg, once daily, of encorafenib. In some embodiments, the method comprises administering to the patient about 350 mg, once daily, of encorafenib. In some embodiments, the method comprises administering to the patient about 400 mg, once daily, of encorafenib. In some embodiments, the method comprises administering to the patient about 450 mg, once daily, of encorafenib. In some embodiments, the method comprises administering to the patient about 500 mg, once daily, of encorafenib. In some embodiments, the method comprises orally administering to the patient, daily, one or more capsules each comprising 75 mg of encorafenib. In some embodiments, the method comprises administering to the patient about 250 mg/m² to about 550 mg/m² of cetuximab every two weeks. In some embodiments, the method comprises administering to the patient about 500 mg/m² of cetuximab every two weeks. In some embodiments, the method comprises administering to the patient a loading dose of about 400 mg/m² of cetuximab for about 120 minutes, followed by administering to the patient about 250 mg/m² of cetuximab (e.g., infused over 60 minutes) weekly. In some embodiments, the colorectal cancer is metastatic colorectal cancer. In some embodiments, the colorectal cancer has a BRAF V600E mutation. In some embodiments, the patient was previously administered at least one prior therapy. In some embodiments, the patient was previously administered one or two prior therapies. In some embodiments, the patient was previously administered leucovorin calcium (folinic acid), fluorouracil, and/or oxaliplatin. In some embodiments, the patient was previously administered leucovorin calcium (folinic acid), fluorouracil, and/or irinotecan hydrochloride. In some embodiments, the patient was previously administered folinic acid, 5-fluorouracil, oxaliplatin and/or irinotecan.

In some embodiments, the method comprises administering to the patient each of the compound represented by Formula (I) and encorafenib for from 1 to 30 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 30 consecutive days, and concurrently cetuximab once weekly. In some embodiments, the method comprises administering to the patient each of the compound represented by Formula (I) and encorafenib for from 1 to 28 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 28 consecutive days, and concurrently cetuximab once weekly. In some embodiments, the method comprises administering to the patient each of the compound represented by Formula (I) and encorafenib for from 1 to 21 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 21 consecutive days, and concurrently cetuximab once weekly. In some embodiments, the method comprises administering to the patient each of the compound represented by Formula (I) and encorafenib for from 1 to 14 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 14 consecutive days, and concurrently cetuximab once weekly. In some embodiments, the method comprises administering to the patient each of the compound represented by Formula (I) and encorafenib for from 1 to 7 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 7 consecutive days, and concurrently cetuximab once weekly.

In some embodiments, the method comprises administering to the patient each of the compound represented by Formula (I) and encorafenib for from 1 to 30 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 7 consecutive days, and concurrently cetuximab once weekly. In some embodiments, the method comprises administering to the patient each of the compound represented by Formula (I) and encorafenib for from 1 to 30 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 14 consecutive days, and concurrently cetuximab once weekly. In some embodiments, the method comprises administering to the patient each of the compound represented by Formula (I) and encorafenib for from 1 to 30 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 21 consecutive days, and concurrently cetuximab once weekly. In some embodiments, the method comprises administering to the patient each of the compound represented by Formula (I) and encorafenib for from 1 to 30 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 28 consecutive days, and concurrently cetuximab once weekly.

In some embodiments, the method comprises administering to the patient each of the compound represented by Formula (I) and encorafenib for from 1 to 21 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 7 consecutive days, and concurrently cetuximab once weekly. In some embodiments, the method comprises administering to the patient each of the compound represented by Formula (I) and encorafenib for from 1 to 21 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 14 consecutive days, and concurrently cetuximab once weekly. In some embodiments, the method comprises administering to the patient each of the compound represented by Formula (I) and encorafenib for from 1 to 21 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 28 consecutive days, and concurrently cetuximab once weekly. In some embodiments, the method comprises administering to the patient each of the compound represented by Formula (I) and encorafenib for from 1 to 21 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 30 consecutive days, and concurrently cetuximab once weekly.

In some embodiments, the method comprises administering to the patient each of the compound represented by Formula (I) and encorafenib for from 1 to 14 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 7 consecutive days, and concurrently cetuximab once weekly. In some embodiments, the method comprises administering to the patient each of the compound represented by Formula (I) and encorafenib for from 1 to 14 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 21 consecutive days, and concurrently cetuximab once weekly. In some embodiments, the method comprises administering to the patient each of the compound represented by Formula (I) and encorafenib for from 1 to 14 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 28 consecutive days, and concurrently cetuximab once weekly. In some embodiments, the method comprises administering to the patient each of the compound represented by Formula (I) and encorafenib for from 1 to 14 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 30 consecutive days, and concurrently cetuximab once weekly.

In some embodiments, the method comprises administering to the patient each of the compound represented by Formula (I) and encorafenib for from 1 to 7 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 14 consecutive days, and concurrently cetuximab once weekly. In some embodiments, the method comprises administering to the patient each of the compound represented by Formula (I) and encorafenib for from 1 to 7 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 21 consecutive days, and concurrently cetuximab once weekly. In some embodiments, the method comprises administering to the patient each of the compound represented by Formula (I) and encorafenib for from 1 to 7 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 28 consecutive days, and concurrently cetuximab once weekly. In some embodiments, the method comprises administering to the patient each of the compound represented by Formula (I) and encorafenib for from 1 to 7 consecutive days, and concurrently cetuximab once weekly, followed by a drug holiday of the compound represented by Formula (I), encorafenib, and/or cetuximab, followed by administering to the patient each of the compound represented by Formula (I) and encorafenib for from 1 to 30 consecutive days, and concurrently cetuximab once weekly.

In some embodiments, the method comprises administering to the patient each of the compound represented by Formula (I) and encorafenib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, and concurrently cetuximab once weekly, followed by a drug holiday of the compound represented by Formula (I), encorafenib, and/or cetuximab, followed by administering to the patient each of the compound represented by Formula (I) and encorafenib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, and concurrently cetuximab once weekly.

In some embodiments, the method comprises administering to the patient each of the compound represented by Formula (I) and encorafenib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, and concurrently cetuximab once weekly, followed by a drug holiday of the compound represented by Formula (I), encorafenib, and/or cetuximab, followed by administering to the patient each of the compound represented by Formula (I) and encorafenib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, and concurrently cetuximab once weekly.

In some embodiments, the drug holiday of the compound represented by Formula (I), encorafenib, and/or cetuximab is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I), encorafenib, and/or cetuximab is a period of 1 week, 2 weeks, 3 weeks, 4 weeks, 5 weeks, or 6 weeks. In some embodiments, the drug holiday of the compound represented by Formula (I), encorafenib, and/or cetuximab is a period of 1-6 weeks. In some embodiments, the drug holiday of the compound represented by Formula (I), encorafenib, and/or cetuximab is a period of 1-4 weeks. In some embodiments, the drug holiday of the compound represented by Formula (I), encorafenib, and/or cetuximab is a period of 1-3 weeks. In some embodiments, the drug holiday of the compound represented by Formula (I), encorafenib, and/or cetuximab is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of encorafenib in the patient. In some embodiments, the drug holiday is about 1-3 times the half-life of encorafenib in the patient. In some embodiments, the drug holiday is about 1-4 times the half-life of encorafenib in the patient. In some embodiments, the drug holiday is about 1-5 times the half-life of encorafenib in the patient. In some embodiments, the drug holiday is about 1-6 times the half-life of encorafenib in the patient. In some embodiments, the drug holiday is about 4-5 times the half-life of encorafenib in the patient. In some embodiments, the drug holiday is about 4 times the half-life of encorafenib in the patient. In some embodiments, the drug holiday is about 5 times the half-life of encorafenib in the patient.

In some embodiments, the drug holiday is about 1-2 times the half-life of cetuximab in the patient. In some embodiments, the drug holiday is about 1-3 times the half-life of cetuximab in the patient. In some embodiments, the drug holiday is about 1-4 times the half-life of cetuximab in the patient. In some embodiments, the drug holiday is about 1-5 times the half-life of cetuximab in the patient. In some embodiments, the drug holiday is about 1-6 times the half-life of cetuximab in the patient. In some embodiments, the drug holiday is about 4-5 times the half-life of cetuximab in the patient. In some embodiments, the drug holiday is about 4 times the half-life of cetuximab in the patient. In some embodiments, the drug holiday is about 5 times the half-life of cetuximab in the patient.

Combinations with Ripretinib

In another embodiment, described herein a method of treating in a gastrointestinal stromal tumor in a patient in need thereof, comprising administering to the patient: (i) a therapeutically effective amount of an inhibitor of ULK1 kinase, an inhibitor of ULK2 kinase, or an inhibitor of ULK1 and ULK2 kinases; and (ii) a therapeutically effective amount of ripretinib.

In another embodiment, described herein is a method of treating in an advanced gastrointestinal stromal tumor in a patient in need thereof, comprising: (i) administering to the patient a therapeutically effective amount of a compound represented by Formula (I):

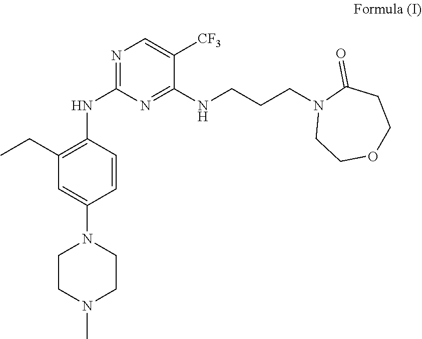 [SMILES: CCc1cc(N2CCN(C)CC2)ccc1Nc1ncc(C(F)(F)F)c(NCCCN2CCOCCC2=O)n1]

or a pharmaceutically acceptable salt thereof; and (ii) orally administering to the patient a therapeutically effective amount of ripretinib. In some embodiments, the method comprises orally administering the compound to the patient. In some embodiments, the method comprises administering to the patient about 20 mg to about 1200 mg, daily, of the compound or pharmaceutically acceptable salt thereof. In some embodiments, the method comprises administering to the patient about 20 mg to about 600 mg, once or twice daily, of the compound or pharmaceutically acceptable salt thereof. In some embodiments, the method comprises administering to the patient about 20 mg to about 400 mg, once or twice daily, of the compound or pharmaceutically acceptable salt thereof. In some embodiments, the method comprises administering to the patient about 50 mg to about 300 mg, twice daily, of the compound or pharmaceutically acceptable salt thereof. In some embodiments, the method comprises administering to the patient about 100 mg to about 800 mg, twice daily, of the compound or pharmaceutically acceptable salt thereof. In some embodiments, the method comprises administering to the patient about 100 mg to about 700 mg, twice daily, of the compound or pharmaceutically acceptable salt thereof. In some embodiments, the method comprises administering to the patient about 100 mg to about 600 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 500 mg, twice daily, of the compound or pharmaceutically acceptable salt thereof. In some embodiments, the method comprises administering to the patient about 100 mg to about 300 mg, twice daily, of the compound or pharmaceutically acceptable salt thereof. In some embodiments, the method comprises administering to the patient about 100 mg to about 200 mg, twice daily, of the compound or pharmaceutically acceptable salt thereof. In some embodiments, the method comprises administering to the patient about 200 mg to about 800 mg, twice daily, of the compound or pharmaceutically acceptable salt thereof. In some embodiments, the method comprises administering to the patient about 200 mg to about 700 mg, twice daily, of the compound or pharmaceutically acceptable salt thereof. In some embodiments, the method comprises administering to the patient about 200 mg to about 600 mg, twice daily, of the compound or pharmaceutically acceptable salt thereof. In some embodiments, the method comprises administering to the patient about 200 mg to about 500 mg, twice daily, of the compound or pharmaceutically acceptable salt thereof. In some embodiments, the method comprises administering to the patient about 200 mg to about 400 mg, twice daily, of the compound or pharmaceutically acceptable salt thereof. In some embodiments, the method comprises administering to the patient about 50 mg, twice daily, of the compound or pharmaceutically acceptable salt thereof. In some embodiments, the method comprises administering to the patient about 60 mg, twice daily, of the compound or pharmaceutically acceptable salt thereof. In some embodiments, the method comprises administering to the patient about 70 mg, twice daily, of the compound or pharmaceutically acceptable salt thereof. In some embodiments, the method comprises administering to the patient about 80 mg, twice daily, of the compound or pharmaceutically acceptable salt thereof. In some embodiments, the method comprises administering to the patient about 90 mg, twice daily, of the compound or pharmaceutically acceptable salt thereof. In some embodiments, the method comprises administering to the patient about 100 mg, twice daily, of the compound or pharmaceutically acceptable salt thereof. In some embodiments, the method comprises administering to the patient about 110 mg, twice daily, of the compound or pharmaceutically acceptable salt thereof. In some embodiments, the method comprises administering to the patient about 120 mg, twice daily, of the compound or pharmaceutically acceptable salt thereof. In some embodiments, the method comprises administering to the patient about 130 mg, twice daily, of the compound or pharmaceutically acceptable salt thereof. In some embodiments, the method comprises administering to the patient about 140 mg, twice daily, of the compound or pharmaceutically acceptable salt thereof. In some embodiments, the method comprises administering to the patient about 150 mg, twice daily, of the compound or pharmaceutically acceptable salt thereof. In some embodiments, the method comprises administering to the patient about 160 mg, twice daily, of the compound or pharmaceutically acceptable salt thereof. In some embodiments, the method comprises administering to the patient about 170 mg, twice daily, of the compound or pharmaceutically acceptable salt thereof. In some embodiments, the method comprises administering to the patient about 180 mg, twice daily, of the compound or pharmaceutically acceptable salt thereof. In some embodiments, the method comprises administering to the patient about 190 mg, twice daily, of the compound or pharmaceutically acceptable salt thereof. In some embodiments, the method comprises administering to the patient about 200 mg, twice daily, of the compound or pharmaceutically acceptable salt thereof. In some embodiments, the method comprises administering to the patient about 250 mg, twice daily, of the compound or pharmaceutically acceptable salt thereof. In some embodiments, the method comprises administering to the patient about 300 mg, twice daily, of the compound or pharmaceutically acceptable salt thereof. In some embodiments, the method comprises administering to the patient about 350 mg, twice daily, of the compound or pharmaceutically acceptable salt thereof. In some embodiments, the method comprises administering to the patient about 400 mg, twice daily, of the compound or pharmaceutically acceptable salt thereof. In some embodiments, the method comprises administering to the patient about 450 mg, twice daily, of the compound or pharmaceutically acceptable salt thereof. In some embodiments, the method comprises administering to the patient about 500 mg, twice daily, of the compound or pharmaceutically acceptable salt thereof. In some embodiments, the method comprises administering to the patient about 550 mg, twice daily, of the compound or pharmaceutically acceptable salt thereof. In some embodiments, the method comprises administering to the patient about 600 mg, twice daily, of the compound or pharmaceutically acceptable salt thereof. In some embodiments, the method comprises administering to the patient about 650 mg, twice daily, of the compound or pharmaceutically acceptable salt thereof. In some embodiments, the method comprises administering to the patient about 700 mg, twice daily, of the compound or pharmaceutically acceptable salt thereof. In some embodiments, the method comprises administering to the patient about 20 mg to about 400 mg, once daily, of the compound or pharmaceutically acceptable salt thereof. In some embodiments, the method comprises administering to the patient about 50 mg to about 300 mg, once daily, of the compound or pharmaceutically acceptable salt thereof. In some embodiments, the method comprises administering to the patient about 100 mg to about 800 mg, once daily, of the compound or pharmaceutically acceptable salt thereof. In some embodiments, the method comprises administering to the patient about 100 mg to about 700 mg, once daily, of the compound or pharmaceutically acceptable salt thereof. In some embodiments, the method comprises administering to the patient about 100 mg to about 600 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 500 mg, once daily, of the compound or pharmaceutically acceptable salt thereof. In some embodiments, the method comprises administering to the patient about 100 mg to about 300 mg, once daily, of the compound or pharmaceutically acceptable salt thereof. In some embodiments, the method comprises administering to the patient about 100 mg to about 200 mg, once daily, of the compound or pharmaceutically acceptable salt thereof. In some embodiments, the method comprises administering to the patient about 200 mg to about 800 mg, once daily, of the compound or pharmaceutically acceptable salt thereof. In some embodiments, the method comprises administering to the patient about 200 mg to about 700 mg, once daily, of the compound or pharmaceutically acceptable salt thereof. In some embodiments, the method comprises administering to the patient about 200 mg to about 600 mg, once daily, of the compound or pharmaceutically acceptable salt thereof. In some embodiments, the method comprises administering to the patient about 200 mg to about 500 mg, once daily, of the compound or pharmaceutically acceptable salt thereof. In some embodiments, the method comprises administering to the patient about 200 mg to about 400 mg, once daily, of the compound or pharmaceutically acceptable salt thereof. In some embodiments, the method comprises administering to the patient about 50 mg, once daily, of the compound or pharmaceutically acceptable salt thereof. In some embodiments, the method comprises administering to the patient about 60 mg, once daily, of the compound or pharmaceutically acceptable salt thereof. In some embodiments, the method comprises administering to the patient about 70 mg, once daily, of the compound or pharmaceutically acceptable salt thereof. In some embodiments, the method comprises administering to the patient about 80 mg, once daily, of the compound or pharmaceutically acceptable salt thereof. In some embodiments, the method comprises administering to the patient about 90 mg, once daily, of the compound or pharmaceutically acceptable salt thereof. In some embodiments, the method comprises administering to the patient about 100 mg, once daily, of the compound or pharmaceutically acceptable salt thereof. In some embodiments, the method comprises administering to the patient about 110 mg, once daily, of the compound or pharmaceutically acceptable salt thereof. In some embodiments, the method comprises administering to the patient about 120 mg, once daily, of the compound or pharmaceutically acceptable salt thereof. In some embodiments, the method comprises administering to the patient about 130 mg, once daily, of the compound or pharmaceutically acceptable salt thereof. In some embodiments, the method comprises administering to the patient about 140 mg, once daily, of the compound or pharmaceutically acceptable salt thereof. In some embodiments, the method comprises administering to the patient about 150 mg, once daily, of the compound or pharmaceutically acceptable salt thereof. In some embodiments, the method comprises administering to the patient about 160 mg, once daily, of the compound or pharmaceutically acceptable salt thereof. In some embodiments, the method comprises administering to the patient about 170 mg, once daily, of the compound or pharmaceutically acceptable salt thereof. In some embodiments, the method comprises administering to the patient about 180 mg, once daily, of the compound or pharmaceutically acceptable salt thereof. In some embodiments, the method comprises administering to the patient about 190 mg, once daily, of the compound or pharmaceutically acceptable salt thereof. In some embodiments, the method comprises administering to the patient about 200 mg, once daily, of the compound or pharmaceutically acceptable salt thereof. In some embodiments, the method comprises administering to the patient about 250 mg, once daily, of the compound or pharmaceutically acceptable salt thereof. In some embodiments, the method comprises administering to the patient about 300 mg, once daily, of the compound or pharmaceutically acceptable salt thereof. In some embodiments, the method comprises administering to the patient about 350 mg, once daily, of the compound or pharmaceutically acceptable salt thereof. In some embodiments, the method comprises administering to the patient about 400 mg, once daily, of the compound or pharmaceutically acceptable salt thereof. In some embodiments, the method comprises administering to the patient about 450 mg, once daily, of the compound or pharmaceutically acceptable salt thereof. In some embodiments, the method comprises administering to the patient about 500 mg, once daily, of the compound or pharmaceutically acceptable salt thereof. In some embodiments, the method comprises administering to the patient about 550 mg, once daily, of the compound or pharmaceutically acceptable salt thereof. In some embodiments, the method comprises administering to the patient about 600 mg, once daily, of the compound or pharmaceutically acceptable salt thereof. In some embodiments, the method comprises administering to the patient about 650 mg, once daily, of the compound or pharmaceutically acceptable salt thereof. In some embodiments, the method comprises administering to the patient about 700 mg, once daily, of the compound or pharmaceutically acceptable salt thereof. In some embodiments, the method comprises administering to the patient about 50 mg, once daily, of ripretinib. In some embodiments, the method comprises administering to the patient about 60 mg, once daily, of ripretinib. In some embodiments, the method comprises administering to the patient about 70 mg, once daily, of ripretinib. In some embodiments, the method comprises administering to the patient about 80 mg, once daily, of ripretinib. In some embodiments, the method comprises administering to the patient about 90 mg, once daily, of ripretinib. In some embodiments, the method comprises administering to the patient about 100 mg, once daily, of ripretinib. In some embodiments, the method comprises administering to the patient about 150 mg, once daily, of ripretinib. In some embodiments, the method comprises administering to the patient about 50 mg, twice daily, of ripretinib. In some embodiments, the method comprises administering to the patient about 60 mg, twice daily, of ripretinib. In some embodiments, the method comprises administering to the patient about 70 mg, twice daily, of ripretinib. In some embodiments, the method comprises administering to the patient about 80 mg, twice daily, of ripretinib. In some embodiments, the method comprises administering to the patient about 90 mg, twice daily, of ripretinib. In some embodiments, the method comprises administering to the patient about 100 mg, twice daily, of ripretinib. In some embodiments, the method comprises administering to the patient about 150 mg, twice daily, of ripretinib. In some embodiments, the method comprises orally administering to the patient, daily, one or more tablets each comprising 50 mg of ripretinib. In some embodiments, the gastrointestinal stromal tumor is an advanced gastrointestinal stromal tumor. In some embodiments, the gastrointestinal stromal tumor is selected from the group consisting of an NF-1-deficient gastrointestinal stromal tumor, a succinate dehydrogenase (SDH)-deficient gastrointestinal stromal tumor, a KIT driven gastrointestinal stromal tumor, and a PDGFRA driven gastrointestinal stromal tumor. In some embodiments, the gastrointestinal stromal tumor has a KIT exon 11 mutation. In some embodiments, the gastrointestinal stromal tumor has a KIT exon 9 mutation, a PDGFRA exon 18 mutation, a PDGFRA exon 12 mutation, or a PDGFRA exon 18 activation loop mutation. In some embodiments, the patient was previously administered at least one tyrosine kinase inhibitors before administration of the ripretinib. In some embodiments, the patient was previously administered one tyrosine kinase inhibitor before administration of the ripretinib. In some embodiments, the patient was previously administered at least two tyrosine kinase inhibitors before administration of the ripretinib. In some embodiments, the patient was previously administered at least three tyrosine kinase inhibitors before administration of the ripretinib. In some embodiments, the patient's gastrointestinal stromal tumor has progressed from, or the patient was intolerant to, a first line administration of imatinib, a second line administration of sunitinib, and a third line administration of regorafenib, or wherein the patient has a documented intolerance to one or more of imatinib, sunitinib and/or regorafenib.

In some embodiments, the method comprises administering to the patient each of the compound represented by Formula (I) and ripretinib for from 1 to 30 consecutive days, followed by a drug holiday of the compound represented by Formula (I) and/or ripretinib, followed by administering to the patient each of the compound represented by Formula (I) and ripretinib for from 1 to 30 consecutive days. In some embodiments, the method comprises administering to the patient each of the compound represented by Formula (I) and ripretinib for from 1 to 28 consecutive days, followed by a drug holiday of the compound represented by Formula (I) and/or ripretinib, followed by administering to the patient each of the compound represented by Formula (I) and ripretinib for from 1 to 28 consecutive days. In some embodiments, the method comprises administering to the patient each of the compound represented by Formula (I) and ripretinib for from 1 to 21 consecutive days, followed by a drug holiday of the compound represented by Formula (I) and/or ripretinib, followed by administering to the patient each of the compound represented by Formula (I) and ripretinib for from 1 to 21 consecutive days. In some embodiments, the method comprises administering to the patient each of the compound represented by Formula (I) and ripretinib for from 1 to 14 consecutive days, followed by a drug holiday of the compound represented by Formula (I) and/or ripretinib, followed by administering to the patient each of the compound represented by Formula (I) and ripretinib for from 1 to 14 consecutive days. In some embodiments, the method comprises administering to the patient each of the compound represented by Formula (I) and ripretinib for from 1 to 7 consecutive days, followed by a drug holiday of the compound represented by Formula (I) and/or ripretinib, followed by administering to the patient each of the compound represented by Formula (I) and ripretinib for from 1 to 7 consecutive days.

In some embodiments, the method comprises administering to the patient each of the compound represented by Formula (I) and ripretinib for from 1 to 30 consecutive days, followed by a drug holiday of the compound represented by Formula (I) and/or ripretinib, followed by administering to the patient each of the compound represented by Formula (I) and ripretinib for from 1 to 7 consecutive days. In some embodiments, the method comprises administering to the patient each of the compound represented by Formula (I) and ripretinib for from 1 to 30 consecutive days, followed by a drug holiday of the compound represented by Formula (I) and/or ripretinib, followed by administering to the patient each of the compound represented by Formula (I) and ripretinib for from 1 to 14 consecutive days. In some embodiments, the method comprises administering to the patient each of the compound represented by Formula (I) and ripretinib for from 1 to 30 consecutive days, followed by a drug holiday of the compound represented by Formula (I) and/or ripretinib, followed by administering to the patient each of the compound represented by Formula (I) and ripretinib for from 1 to 21 consecutive days. In some embodiments, the method comprises administering to the patient each of the compound represented by Formula (I) and ripretinib for from 1 to 30 consecutive days, followed by a drug holiday of the compound represented by Formula (I) and/or ripretinib, followed by administering to the patient each of the compound represented by Formula (I) and ripretinib for from 1 to 28 consecutive days.

In some embodiments, the method comprises administering to the patient each of the compound represented by Formula (I) and ripretinib for from 1 to 21 consecutive days, followed by a drug holiday of the compound represented by Formula (I) and/or ripretinib, followed by administering to the patient each of the compound represented by Formula (I) and ripretinib for from 1 to 7 consecutive days. In some embodiments, the method comprises administering to the patient each of the compound represented by Formula (I) and ripretinib for from 1 to 21 consecutive days, followed by a drug holiday of the compound represented by Formula (I) and/or ripretinib, followed by administering to the patient each of the compound represented by Formula (I) and ripretinib for from 1 to 14 consecutive days. In some embodiments, the method comprises administering to the patient each of the compound represented by Formula (I) and ripretinib for from 1 to 21 consecutive days, followed by a drug holiday of the compound represented by Formula (I) and/or ripretinib, followed by administering to the patient each of the compound represented by Formula (I) and ripretinib for from 1 to 28 consecutive days. In some embodiments, the method comprises administering to the patient each of the compound represented by Formula (I) and ripretinib for from 1 to 21 consecutive days, followed by a drug holiday of the compound represented by Formula (I) and/or ripretinib, followed by administering to the patient each of the compound represented by Formula (I) and ripretinib for from 1 to 30 consecutive days.

In some embodiments, the method comprises administering to the patient each of the compound represented by Formula (I) and ripretinib for from 1 to 14 consecutive days, followed by a drug holiday of the compound represented by Formula (I) and/or ripretinib, followed by administering to the patient each of the compound represented by Formula (I) and ripretinib for from 1 to 7 consecutive days. In some embodiments, the method comprises administering to the patient each of the compound represented by Formula (I) and ripretinib for from 1 to 14 consecutive days, followed by a drug holiday of the compound represented by Formula (I) and/or ripretinib, followed by administering to the patient each of the compound represented by Formula (I) and ripretinib for from 1 to 21 consecutive days. In some embodiments, the method comprises administering to the patient each of the compound represented by Formula (I) and ripretinib for from 1 to 14 consecutive days, followed by a drug holiday of the compound represented by Formula (I) and/or ripretinib, followed by administering to the patient each of the compound represented by Formula (I) and ripretinib for from 1 to 28 consecutive days. In some embodiments, the method comprises administering to the patient each of the compound represented by Formula (I) and ripretinib for from 1 to 14 consecutive days, followed by a drug holiday of the compound represented by Formula (I) and/or ripretinib, followed by administering to the patient each of the compound represented by Formula (I) and ripretinib for from 1 to 30 consecutive days.

In some embodiments, the method comprises administering to the patient each of the compound represented by Formula (I) and ripretinib for from 1 to 7 consecutive days, followed by a drug holiday of the compound represented by Formula (I) and/or ripretinib, followed by administering to the patient each of the compound represented by Formula (I) and ripretinib for from 1 to 14 consecutive days. In some embodiments, the method comprises administering to the patient each of the compound represented by Formula (I) and ripretinib for from 1 to 7 consecutive days, followed by a drug holiday of the compound represented by Formula (I) and/or ripretinib, followed by administering to the patient each of the compound represented by Formula (I) and ripretinib for from 1 to 21 consecutive days. In some embodiments, the method comprises administering to the patient each of the compound represented by Formula (I) and ripretinib for from 1 to 7 consecutive days, followed by a drug holiday of the compound represented by Formula (I) and/or ripretinib, followed by administering to the patient each of the compound represented by Formula (I) and ripretinib for from 1 to 28 consecutive days. In some embodiments, the method comprises administering to the patient each of the compound represented by Formula (I) and ripretinib for from 1 to 7 consecutive days, followed by a drug holiday of the compound represented by Formula (I) and/or ripretinib, followed by administering to the patient each of the compound represented by Formula (I) and ripretinib for from 1 to 30 consecutive days.

In some embodiments, the method comprises administering to the patient each of the compound represented by Formula (I) and ripretinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days, followed by a drug holiday of the compound represented by Formula (I) and/or ripretinib, followed by administering to the patient each of the compound represented by Formula (I) and ripretinib for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days.

In some embodiments, the method comprises administering to the patient each of the compound represented by Formula (I) and ripretinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days, followed by a drug holiday of the compound represented by Formula (I) and/or ripretinib, followed by administering to the patient each of the compound represented by Formula (I) and ripretinib for 1 day, 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, 14 consecutive days, 15 consecutive days, 16 consecutive days, 17 consecutive days, 18 consecutive days, 19 consecutive days, 20 consecutive days, 21 consecutive days, 22 consecutive days, 23 consecutive days, 24 consecutive days, 25 consecutive days, 26 consecutive days, 27 consecutive days, 28 consecutive days, 29 consecutive days, 30 consecutive days, or 31 consecutive days.

In some embodiments, the drug holiday of the compound represented by Formula (I) and/or ripretinib is a period of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, or 31 days. In some embodiments, the drug holiday of the compound represented by Formula (I) and/or ripretinib is a period of 1 week, 2 weeks, 3 weeks, 4 weeks, 5 weeks, or 6 weeks. In some embodiments, the drug holiday is a period of 1-6 weeks. In some embodiments, the drug holiday of the compound represented by Formula (I) and/or ripretinib is a period of 1-4 weeks. In some embodiments, the drug holiday of the compound represented by Formula (I) and/or ripretinib is a period of 1-3 weeks. In some embodiments, the drug holiday is a period of 1-2 weeks.

In some embodiments, the drug holiday is about 1-2 times the half-life of the compound represented by Formula (I) in the patient. In some embodiments, the drug holiday is about 1-3 times the half-life of the compound represented by Formula (I) in the patient. In some embodiments, the drug holiday is about 1-4 times the half-life of the compound represented by Formula (I) in the patient. In some embodiments, the drug holiday is about 1-5 times the half-life of the compound represented by Formula (I) in the patient. In some embodiments, the drug holiday is about 1-6 times the half-life of the compound represented by Formula (I) in the patient. In some embodiments, the drug holiday is about 4-5 times the half-life of the compound represented by Formula (I) in the patient. In some embodiments, the drug holiday is about 4 times the half-life of the compound represented by Formula (I) in the patient. In some embodiments, the drug holiday is about 5 times the half-life of the compound represented by Formula (I) in the patient.

In some embodiments, the drug holiday is about 1-2 times the half-life of ripretinib in the patient. In some embodiments, the drug holiday is about 1-3 times the half-life of ripretinib in the patient. In some embodiments, the drug holiday is about 1-4 times the half-life of ripretinib in the patient. In some embodiments, the drug holiday is about 1-5 times the half-life of ripretinib in the patient. In some embodiments, the drug holiday is about 1-6 times the half-life of ripretinib in the patient. In some embodiments, the drug holiday is about 4-5 times the half-life of ripretinib in the patient. In some embodiments, the drug holiday is about 4 times the half-life of ripretinib in the patient. In some embodiments, the drug holiday is about 5 times the half-life of ripretinib in the patient.

### Combination Therapy

Compounds described herein, e.g., a compound of Formula I as defined herein, can be administered in combination with one or more additional therapeutic agents to treat a disorder described herein, such as a cancer described herein. For example, provided in the present disclosure is a pharmaceutical composition comprising a compound described herein, e.g., a compound of Formula I as defined herein, one or more additional therapeutic agents, and a pharmaceutically acceptable excipient. In some embodiments, a compound of Formula I as defined herein and one additional therapeutic agent is administered. In some embodiments, a compound of Formula I as defined herein and two additional therapeutic agents are administered. In some embodiments, a compound of Formula I as defined herein and three additional therapeutic agents are administered. Combination therapy can be achieved by administering two or more therapeutic agents, each of which is formulated and administered separately. For example, a compound of Formula I as defined herein and an additional therapeutic agent can be formulated and administered separately. Combination therapy can also be achieved by administering two or more therapeutic agents in a single formulation, for example a pharmaceutical composition comprising a compound of Formula I as one therapeutic agent and one or more additional therapeutic agents. For example, a compound of Formula I as defined herein and an additional therapeutic agent can be administered in a single formulation. Other combinations are also encompassed by combination therapy. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination using different sequencing of the component agents. For example, if agent X and agent Y are used in a combination, one could administer them sequentially in any combination one or more times, e.g., in the order X-Y-X, X-X-Y, Y-X-Y, Y-Y-X, X-X-Y-Y, etc.

### EXAMPLES

### Example 1. A Phase 1/2, First-In-Human Study of DCC-3116 as Monotherapy and in Combination with RAS/MAPK Pathway Inhibitors in Patients with Advanced or Metastatic Solid Tumors with RAS/MAPK Pathway Mutations

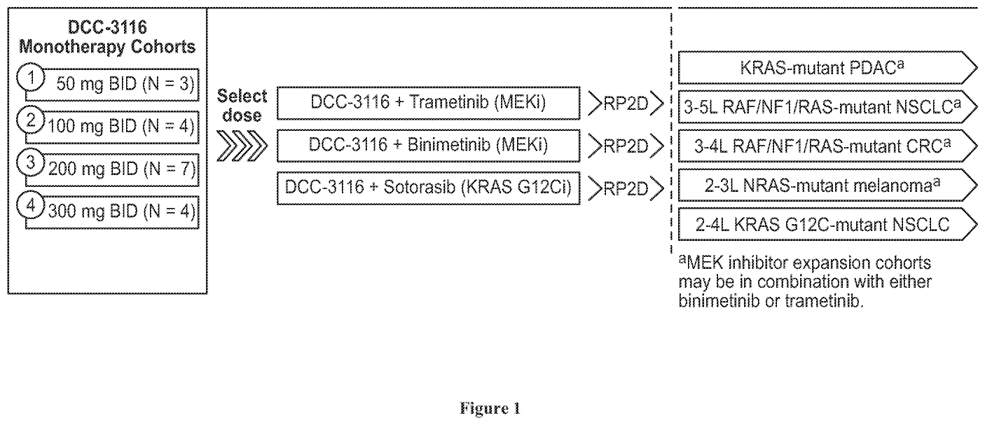

This is a Phase 1/2, multicenter, open label, first in human (FIH) study of DCC-3116 as monotherapy, and in combination with trametinib, binimetinib, or sotorasib in patients with advanced or metastatic solid tumors with RAS/MAPK pathway mutation. This study is composed of 2 parts: Dose Escalation Phase (Part 1), in which approximately 100 participants will be enrolled, and Dose Expansion Phase (Part 2), in which approximately 223 participants will be enrolled. DCC-3116 will be administered to participants orally in 28-day cycles as monotherapy (Dose Escalation Cohort A), in combination with trametinib, in combination with binimetinib, or in combination with sotorasib (Dose Escalation Cohort B, C, and D, respectively) according to the assigned dose and regimen. Participants may remain on treatment until they develop progressive disease as assessed by the Investigator, experience unacceptable toxicity, or withdraw consent. A summary of the study design is provided as FIG. 1.

### Part 1

In Part 1, participants with RAS, NF1, or RAF pathway mutations who progressed despite standard therapies or for whom conventional therapy is not considered effective or tolerable as judged by the Investigator are enrolled. The starting dose of DCC-3116 is defined based on evaluation of nonclinical toxicology studies and dose escalation in all cohorts of Part 1 will use a modified 3+3 design. In order to ensure the safety of participants, the monotherapy dose escalation cohort was evaluated first. The first cohorts of the combination dose escalation will open after the Recommended Phase 2 Dose and/or maximum tolerated dose of DCC-3116 as monotherapy (RP2D-M or MTD-M, respectively) is determined and will use a dose level of DCC-3116 at least one dose level below the MTD. The maximum tolerated dose (MTD) will be defined as the highest dose level at which no more than 1 of at least 6 DLT evaluable participants, or no more than 30% if more than 6 participants are treated, experiences a DLT in Cycle 1 during dose escalation. Dose escalation may stop in the absence of dose-limited toxicities (DLTs) based on available safety, pharmacokinetic (PK) and pharmacodynamic (PD) analyses. In this case the highest dose evaluated in at least 6 participants may be moved from monotherapy to combination therapy escalation and from combination therapy escalation to expansion.

The determination of the Recommended Phase 2 Dose (RP2D) may be based on safety and tolerability of at least 3 participants if declared at a dose lower than the MTD. For confirmation of the RP2D, dose levels may be backfilled to approximately 15 participants per selected dose in monotherapy and combination to further characterize the safety and tolerability, PK, and PD profile. The RP2D will be declared based on safety, tolerability, and available PK and PD data and will not exceed the MTD.

Trametinib will be started at the approved dose level (2 mg once daily [QD]), as efficacy in melanoma and other indications has been demonstrated at this dose level. A lower dose of trametinib (1.5 or 1 mg QD) in combination with DCC-3116 may be evaluated based on emerging safety data. Binimetinib will be started at the dose approved in combination with encorafenib (45 mg twice daily [BID]). Lower doses may be evaluated based on emerging safety data (this is generally limited to 30 mg BID, but 15 mg BID may be considered based on safety, PK, and PD analyses). Based on published literature, sotorasib will be started at the dose level of 240 mg QD since this dose is expected to reach similar exposure as the approved dose of 960 mg QD but may have a lower risk for gastrointestinal adverse events. Additional sotorasib doses may be evaluated, including a reduced dose of 120 mg QD and up to a maximum of 960 mg QD. The dose of DCC-3116 in combination with trametinib, binimetinib, or sotorasib may be decreased in at least 25% reduction steps or increased from at least one dose level below the MTD in monotherapy (MTD-M) to the MTD-M or above in up to 50% increments, guided by PK, PD and safety analyses according to the same 3+3 dose escalation rules as in monotherapy escalation. The RP2D of the combination with trametinib, binimetinib, or sotorasib (RP2D-CT, RP2D-CB, or RP2D-CS, respectively) will be determined in at least 3 and up to approximately 15 participants prior to initiation of the Dose Expansion Phase (Part 2) and will be no higher than the MTD-CT, MTD-CB, and MTD-CS, respectively, defined as the highest dose level at which a DLT rate of no higher than 30% occurs in at least 6 DLT evaluable participants. Refer to FIG. 1, Table 7, Table 8, and Table 9 for dose escalation of DCC-3116 as monotherapy and in combination with trametinib or binimetinib or sotorasib, respectively.

### Monotherapy Studies Results

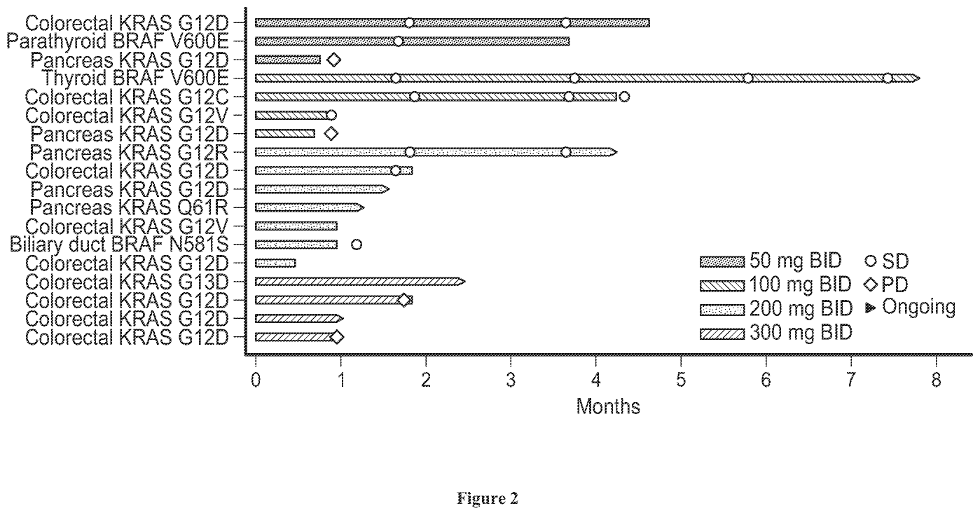

Results from the monotherapy study were obtained. Treatment duration and results of response assessments are provided in FIG. 2. The best overall response was confirmed as stable disease in 4 of 14 response-evaluable participants. The disease control rate at Week 16 was 31% while accounting for 4 of 13 response-evaluable participants, and 29% while accounting for 4 of 14 response-evaluable participants.

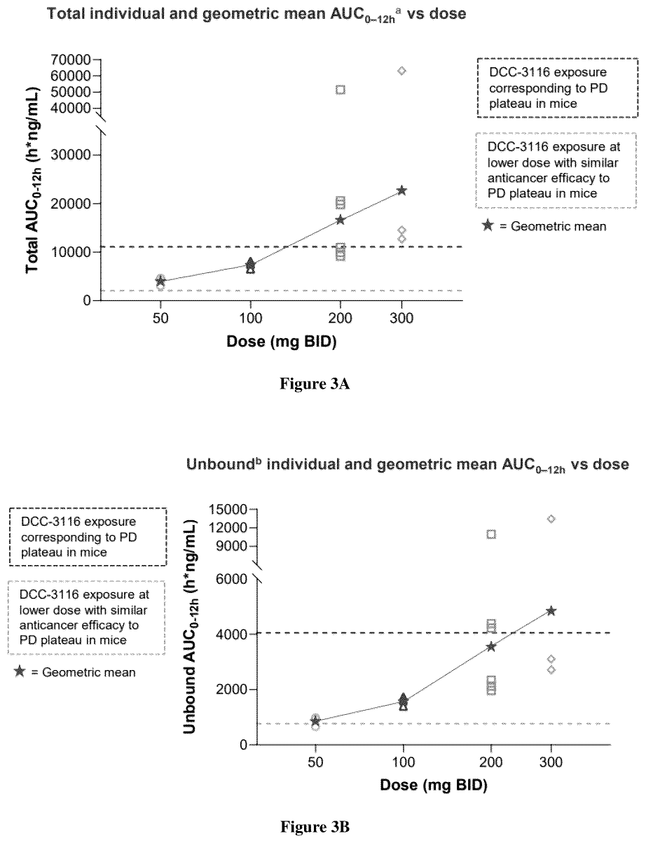

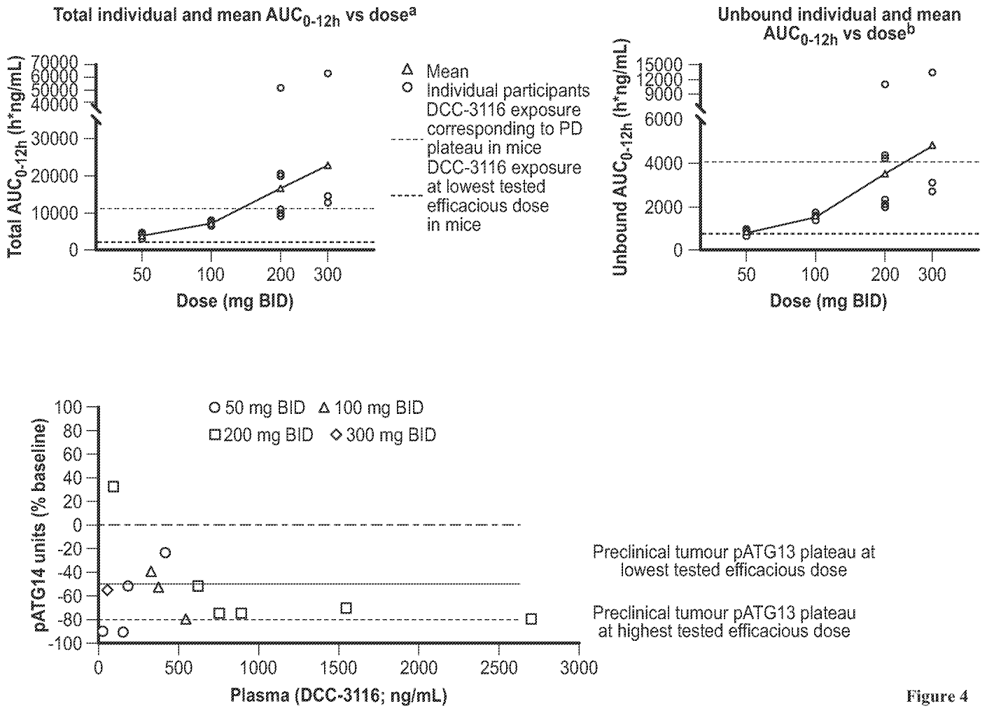

Total and unbound DCC-3116 area under the curve (AUC) results at Cycle 1 Day 5 (C1D15) were obtained as shown in FIGS. 3A and 3B. At DCC-3116 doses greater than or equal to 100 mg twice daily (BID), total (FIG. 3A) and calculated unbound (FIG. 3B) DCC-3116 AUC at C1D15 exceeded efficacious exposures in MiaPaca-2 xenograft mice administered DCC-3116 in combination with trametinib. DCC-3116 AUC appeared to increase proportionally to dose across 50-300 mg BID.

Further, pATG14 in PBMCs according to predose trough exposure were evaluated for Cycle 1 Day 5 and beyond as shown in FIG. 4.

Decreases in ATG14 or ATG13 phosphorylation equally indicate inhibition of ULK1/2 kinase. The assay for the phase 1 study was optimized for pATG14 while the preclinical assay used pATG13. Inhibition of ULK1/2 kinase by these measures reached levels in PBMCs that were seen preclinically in tumors at doses associated with anticancer efficacy in combination with trametinib. The PD plateau in PBMCs in the phase 1 study is similar as the preclinical PD plateau in tumors.

The results show that DCC-3116 had an acceptable safety profile given at doses between 50 and 300 mg BID. No DLTs or treatment-related serious treatment emergent adverse events (TEAEs) were reported, and reversible asymptomatic related Grade 3 ALT increased that led to dose interruption and reduction was observed in 1 participant each at the 200 mg BID and 300 mg BID dose levels. Otherwise, treatment-related TEAEs were reported with low grades (Grade 1 or 2). DCC-3116 exposures appeared to increase dose proportionally across dose levels and at the 100 mg BID dose level, exposures already exceeded exposures associated with anticancer activity in combination with trametinib in preclinical studies. As monotherapy, DCC-3116 resulted in a disease control rate (DCR) at Week 16 of 31% with stable disease as best overall response rate (BOR) in 4 of 13 response evaluable participants. ULK1/2 kinase inhibition was confirmed by suppression of phosphorylation of ATG14 in PBMCs and in a range associated with anticancer efficacy in combination with trametinib in preclinical studies where it was assessed in tumours with an equal readout (phosphorylation of ATG13). The 100 mg-300 mg BID dose cohorts are being expanded to further characterize safety, PK, and PD and inform selection of the DCC-3116 starting dose for combination dose-finding with MEK inhibitors trametinib and binimetinib and the KRAS G12C inhibitor sotorasib.

In the dose escalation study, an active N-demethylated metabolite of DCC-3116 (Compound A) was found to be present in about 5% of total drug measured.

### Summary of Initial Single Agent Phase 1 Study Results

DCC-3116 was well tolerated at doses from 50 mg BID to 300 mg BID, which achieved exposure and ULK1/2 inhibition associated with anticancer efficacy with MEK inhibitors in preclinical studies. Further, treatment-emergent adverse events were mainly Grade 1 or 2, except for related asymptomatic and reversible Grade 3 ALT increases.

Additionally, DCC-3116 exposure appeared to increase dose proportionally across 50 mg BID to 300 mg BID. All doses achieved exposure and ULK1/2 inhibition associated with efficacy in preclinical studies. No dose-limiting toxicities (DLTs) or treatment-related serious adverse events (SAEs) were observed. Further, a maximum tolerated dose was not reached. The monotherapy results demonstrated stable disease as best overall response.

Table 1 summarizes pharmacokinetic parameter estimates on Cycle 1 Day 15 for participants receiving DCC-3116 as monotherapy (Cohorts A1-4). The mean accumulation ratio of AUC was approximately 1.8- to 2-fold following 15 days of DCC-3116 BID dosing relative to Day 1. The mean AUC ratio of the active metabolite to the parent drug, DCC-3116 was approximately 3- to 3.6-fold across doses. Following the exclusion of profiles that appeared to exhibit continued absorption beyond the sampling interval (i.e., where tₘₐₓ≥10 h), the tₘₐₓ ranged from 2 to 8 hours on Cycle 1 Day 15.

TABLE 1

| | Cohort A1 | Cohort A2 | Cohort A3 | Cohort A4 |
|---|---|---|---|---|
| | 50 mg BID | 100 mg BID | 200 mg BID | 300 mg BID |
| Parameter | (N = 3) | (N = 7)^{a} | (N = 6)^{b} | (N = 6)^{c} |
| tₘₐₓ (hr) | 2 (2, 8) | 4 (2, 8) | 3 (2, 6) | 2 (2, 6) |
| (Median [Min, Max]) | | | | |
| Cₘₐₓ (ng/mL) | 724 (9) | 1140 (30) | 2220 (95) | 2040 (77) |
| (GM [CV %]) | | | | |
| AUC_{0-10 h} (hr*ng/mL) | 3540 (17) | 8540 (36) | 13800 (84) | 13700 (86) |
| (GM [CV %]) | | | | |
| AUC_{0-12 h} (hr*ng/mL) | 3980 (21) | 9840 (40) | 15600 (83) | 16400 (85) |
| (GM [CV %])^{d} | | | | |
| C_{trough} (ng/mL) | 118 (290) | 542 (66) | 751 (82) | 1109 (124) |
| (GM [CV %]) | | | | |
| MPR_{AUC0-10 h} (%) | 3.64 (6) | 2.93 (40) | 3.64 (26) | 3.64 (35) |
| (GM [CV %]) | | | | |
| AR_{AUC0-10 h} (%) | 1.81, 1.90 | 2.05 (50) | 2.18 (42) | 1.57 (43) |
| (GM [CV %]) | | | | |

Abbreviations: AUC_{0-10 h} = area under the plasma concentration-time curve at 0-10 hours; AR = accumulation ratio calculated as Day 15 AUC_{0-10 h}/Day 1 AUC_{0-10 h}; Cₘₐₓ = maximum plasma concentration; C_{trough} = predose plasma concentration CV = coefficient of variation; GM = geometric mean; MPR: metabolite AUC_{0-10 h}/parent AUC_{0-10 h} ratio; N = sample size.
^{a}N = 6 for tmax and Cmax since it was not reported for one participant; N = 6 for ARauc0-10 h
^{b}Two participants were excluded: one due to missed doses and one due to potential assay error
^{c}One participant was excluded due to missed doses; N = 5 for tₘₐₓ and Cₘₐₓ since it was not reported for 105-004; N = 3 for ARauc0-10 h
^{d}AUC_{0-12 h} was calculated by imputing the predose concentration to 12 hours.

Note: In cases where there ≤2 reportable estimates, geometric means cannot be calculated and individual estimates are reported instead.

### Part 2

The Dose Expansion Phase (Part 2) will have 5 expansion cohorts. Start of enrollment into each cohort will be determined by the Sponsor. Expansion Cohorts 1 to 3 will evaluate safety and preliminary efficacy of DCC-3116 in combination with trametinib or with binimetinib if the PK, PD, safety, and preliminary efficacy profile during dose escalation favor a combination with this MEK inhibitor: Expansion Cohort 1 will enroll participants with advanced or metastatic pancreatic ductal adenocarcinoma (PDAC) (with a documented mutation in KRAS), Expansion Cohort 2 will enroll participants with non-small cell lung cancer (NSCLC) (with a documented mutation in KRAS, NRAS, NF1 or BRAF), and Expansion Cohort 3 will enroll participants with colorectal cancer (CRC) (with a documented mutation in KRAS, NRAS, NF1, or BRAF). Expansion Cohort 4 will enroll participants with melanoma (with a documented mutation in NRAS) and will evaluate safety and preliminary efficacy of DCC-3116 in combination with binimetinib or with trametinib if the PK, PD, safety, and preliminary efficacy profile during dose escalation favor a combination with this MEK inhibitor. Expansion Cohort 5 will evaluate safety and preliminary efficacy of DCC-3116 in combination with sotorasib in participants with NSCLC (with a documented mutation in KRAS G12C).

### Primary Endpoints

For the Dose Escalation Phase (Part 1), primary endpoints include: safety endpoints including dose-limiting toxicities (DLTs), treatment-emergent adverse events (TEAEs), serious adverse events (SAEs), dose reduction, interruption, or discontinuation of study drug due to toxicity.

For Dose Expansion Phase (Part 2): the primary endpoint is efficacy, more particularly the objective response rate (ORR) based on Investigator Assessment, defined as the proportion of participants who achieve confirmed complete response (CR) or partial response (PR) per RECIST v1.1.

### Secondary Endpoints

For the Dose Escalation Phase (Part 1), secondary endpoints include the following endpoints for assessing antitumor activity of DCC-3116: ORR; duration of response (DOR, for patients who achieve confirmed CR or PR, defined as the time interval from the time that the measurement criteria are first met for CR or PR [whichever is first recorded] until the first date that the progressive disease is objectively documented or death, whichever occurs first); disease Control Rate (DCR) at 16, 24, and 32 weeks (defined as the proportion of participants who achieve CR, PR, or stable disease [SD] at the specified time point per RECIST v1.1); time to response (defined as the time from initiation of treatment until the first assessment demonstrating CR or PR per RECIST v1.1); progression-free survival (PFS; defined as the time from initiation of treatment until documented disease progression per RECIST v1.1 or death whichever occurs first). Secondary endpoints also include endpoints for assessing pharmacokinetics (PK). PK endpoints will be evaluated for DCC-3116 as monotherapy, for DCC-3116 and trametinib when administered in combination, for DCC-3116 and binimetinib when administered in combination, and for DCC-3116 and sotorasib when administered in combination. These primary PK parameters include, but are not limited to the following: time to maximum observed concentration (tmax); maximum observed concentration (Cmax); minimum observed concentration (Cmin); area under the concentration-time curve (AUC).

Dose Expansion Phase (Part 2): Efficacy: Endpoints assessing efficacy of DCC-3116 and trametinib when administered in combination, DCC-3116 and binimetinib when administered in combination, and DCC-3116 and sotorasib when administered in combination include: DOR; DCR at 16, 24, and 32 weeks; time to response; PFS; overall survival (OS; defined as the time from initiation of treatment until death). Pharmacokinetic (PK) endpoints for DCC-3116 and trametinib when administered in combination, for DCC-3116 and binimetinib when administered in combination, and DCC-3116 and sotorasib when administered in combination, are evaluated. These PK parameters include, but are not limited to: tmax, Cmax, Cmin, and AUC. Safety endpoints include the following: TEAEs, SAEs, dose reduction, interruption, or discontinuation of study drugs due to toxicity.

### Exploratory Endpoints

Exploratory endpoints in both Dose Escalation Phase (Part 1) and Dose Expansion Phase (Part 2) are as follows: baseline levels and change in select blood, plasma, hair follicle (in Dose Expansion Phase [Part 2] only), and tumor tissue biomarkers when DCC-3116 is administered as monotherapy, in combination with trametinib, in combination with binimetinib, and in combination with sotorasib; association of genetic variations in the population with differences in PK, PD, efficacy, tolerability, and/or safety; Cmax, Cmin and AUC of Compound A; metabolic profile of DCC-3116 in human plasma; participant-reported symptoms and Health-Related Quality of Life (HRQOL) scores, assessed with the European Organisation for Research and Treatment of Cancer Quality of Life Questionnaire for Cancer 30-item (EORTC QLQ-C30) (v3.0) and GP5 burden-of-side-effects question, a part of the Functional Assessment of Chronic Illness Therapy (FACIT) Functional Assessment of Cancer Therapy-General (FACT-G) questionnaire (Part 2 only).

### Example 2. A Phase 1/2, Phase 1/2, Multi-Center, Open-Label Study to Evaluate Safety and Preliminary Activity of DCC-3116 in Combination with Anticancer Therapies

This is a global Phase 1/2, multi-center, open-label study to evaluate safety and preliminary activity of DCC-3116 in combination with anticancer therapies supported by a strong scientific rationale. Exemplary combination therapies that will be investigated include encorafenib/cetuximab or ripretinib. To facilitate efficient assessment of the clinical promise observed for preclinically validated novel DCC-3116 combinations, the study will use a master protocol design. This consists of modules specific to each drug combination along with an overarching core protocol, which contains elements common to every module. The study modules will generally be conducted in two parts, Safety/Dosefinding (Part 1) and Expansion (Part 2).

Safety/Dose-finding (Part 1): The objective of Safety/Dose-finding (Part 1) is to assess the safety of combining DCC-3116 at the monotherapy recommended Phase 2 dose (RP2D) established in a first in human (FIH) Phase 1/2 study (Example 1) with the module-specific combination partner to determine the combination RP2D and/or the combination maximum tolerated dose (MTD). Safety, pharmacokinetics (PK), and pharmacodynamics (PD) of the combination will be assessed. The module-specific combination partner will be started at an established effective dose and adjusted as needed based on safety, PK, and PD analysis.

A Bayesian optimal interval (BOIN) design will be used since it has shown greater precision at identifying the MTD than the traditional 3+3 design without sacrificing safety. Further, the BOIN design has shown equivalent precision and safety compared to model-based designs, such as the continual reassessment method (CRM). The BOIN design can be more easily implemented with prespecified escalation and de-escalation rules based on the number of evaluable participants and number of participants with dose limiting toxicities (DLTs).

Expansion (Part 2): The objective of Expansion (Part 2) is to evaluate the preliminary anticancer activity and further characterize the safety of DCC-3116 combinations in cancers that use ULK1/2 activity as an escape mechanism. Histology-specific consensus criteria for response assessment (defined in respective modules) will be used as the primary endpoint in a 2-stage design with non-binding futility and efficacy thresholds to assess combinations based on histology-specific benchmarks and clinically meaningful goals. Inhibition of ULK may increase response rate and/or duration of response as well as confer improved stabilization of disease. A design with non-binding futility and efficacy thresholds without mandatory interruption of accrual after Stage 1 will be used to allow for comprehensive benefit analysis in decisions to continue expansions into Stage 2.

### Encorafenib/Cetuximab

The starting dose of DCC-3116 will be the RP2D-monotherapy from the study of Example 1, or a lower dose. Additional experience from DCC-3116 in combination with other anticancer therapies may be considered when selecting the DCC-3116 starting dose. Encorafenib will be started in combination with DCC-3116 and cetuximab at the approved dose level of 300 mg QD. Cetuximab will be started in combination with DCC-3116 and encorafenib at a dose level of 500 mg/m² administered as a 120-minute intravenous (IV) infusion every 2 weeks (Q2W) beginning on C1D1. The Q2W 500 mg/m² dosage regimen of cetuximab was approved in 2021 as single agent and in combination with chemotherapy based on the totality of evidence generated using population pharmacokinetic (pop-PK) modeling analyses, a clinical meta-analyses of published literature, and real world evidence, which showed overlapping concentration-time profiles and similar observed efficacy and safety profiles between cetuximab 500 mg/m² Q2W and cetuximab 250 mg/m² QW. While the Cₘᵢₙ at steady state with cetuximab 500 mg/m² Q2W was approximately 23-25% lower than that of cetuximab 250 mg/m² QW, it was deemed unlikely to be of clinical significance. Based on emerging safety, PK, PD, and preliminary efficacy data, the dose of DCC-3116 will be adjusted and additional encorafenib and/or cetuximab doses or schedules, in combination with DCC-3116, may be evaluated in subsequent cohorts in Safety/Dose-finding (Part 1) before defining an RP2D A1 (DCC-3116 in combination with encorafenib and cetuximab). Alternate dosing may be identified for potential future evaluation in additional expansion cohorts.

Expansion Cohort AE1: The RP2D A1 for the combination of DCC-3116 with encorafenib and cetuximab will be based on the safety and tolerability, PK, PD, and preliminary efficacy profile from Safety/Dose-finding (Part 1).

### Ripretinib

Safety/Dose-finding (Part 1): The starting dose of DCC-3116 will be the RP2D-monotherapy from the FIH study of Example 1, or a lower dose. Additional experience from DCC-3116 in combination with other anticancer therapies may be considered when selecting the DCC-3116 starting dose. Ripretinib will be started at 150 mg QD, the dose approved for 4^{th}-line GIST, in combination with DCC-3116. An additional ripretinib dosing schedule of 150 mg BID may be evaluated which has demonstrated safety and efficacy, including in participants of prior studies who experienced progression on ripretinib 150 mg QD. DCC-3116 doses that have not resulted in DLTs in at least 6 DLT evaluable participants or that are at least one dose level below the MTD in combination with ripretinib 150 mg QD may be used as starting dose for combination with ripretinib 150 mg BID. Based on emerging safety, PK, PD, and preliminary efficacy data, the dose of DCC-3116 will be adjusted and additional ripretinib doses may be evaluated in subsequent cohorts in combination with DCC-3116 as part of Safety/Dose-finding (Part 1) before defining a RP2D B1 (combination of DCC-3116 with ripretinib QD). Alternate dosing may be identified for potential future evaluation in additional expansion cohorts.

Expansion Cohort BE1: The RP2D B1 for the combination of DCC-3116 with ripretinib QD will be based on the safety and tolerability, PK, PD, and preliminary efficacy profile from Safety/Dosefinding (Part 1).

### Example 3. Combination Studies of DCC-3116 and Trametinib

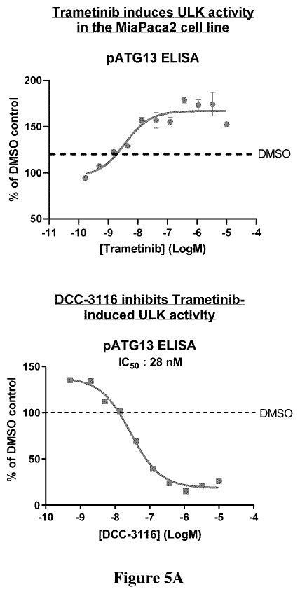

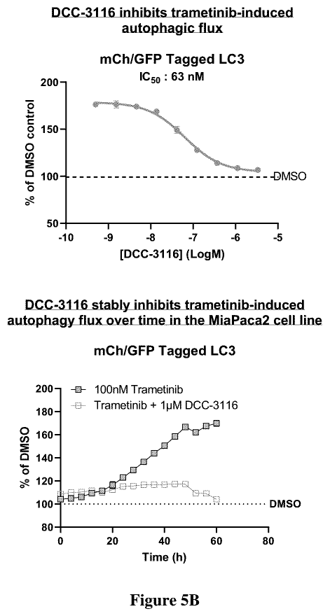

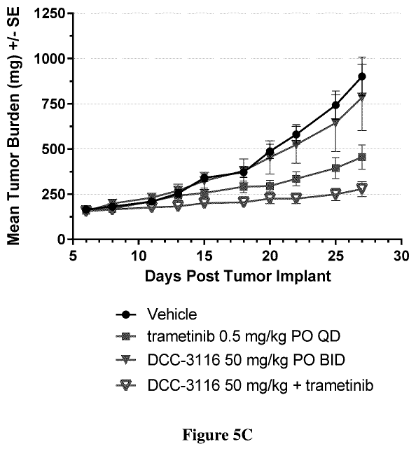

Combinations studies of DCC-3116 and trametinib were evaluated. For example, DCC-3116 was shown to combine with trametinib in KRAS^{G12C}-Mutated pancreatic cancer, as shown in FIGS. 5A, 5B, and 5C.

### Protocol for FIGS. 5A, 5B, and 5C

### Cell Culture:

MiaPaca2 cells (ATCC #CRL-1420) were grown in DMEM medium containing 10% FBS, 1% penicillin-streptomycin-L-Glutamine and 2.5% Horse serum (ThermoFisher #16050-114).

pATG13 ELISA:

Cells were cultured in assay plates and titrated DCC-3116 was added to each well with or without titrated trametinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or trametinib or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time. In vivo efficacy models:

Female nude mice (Hsd:Athymic Nude-Foxn1^{nu}; Envigo; 8-9 weeks old) were inoculated subcutaneously in the right high axilla with 1.0×10⁷ cells in Dulbecco's Phosphate buffered saline mixed with an equal volume of Matrigel. When tumor burdens reached 159 mm³ on average on day 6, mice were randomly assigned into groups such that the mean tumor burden for all groups was within 10% of the overall mean tumor burden for the study population. Groups were treated on days 6-27 as follows: Vehicle control (0.5% CMC/0.1% Tw80/0.45% NaCl), Trametinib 0.5 mgkg PO QD, DCC-3116 50 mg/kg PO BID or the combination of Trametinib 0.5 mg/kg PO QD+DCC-3116 50 mg/kg PO BID.

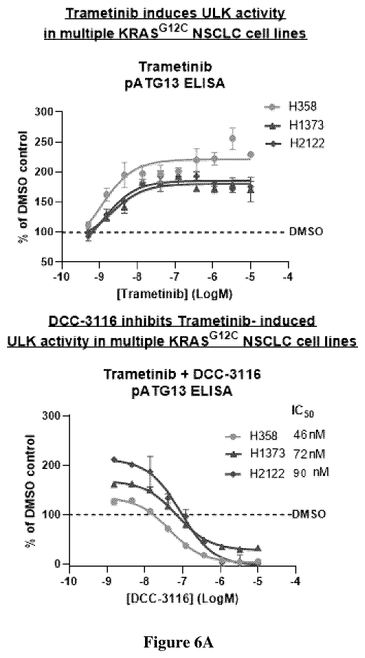

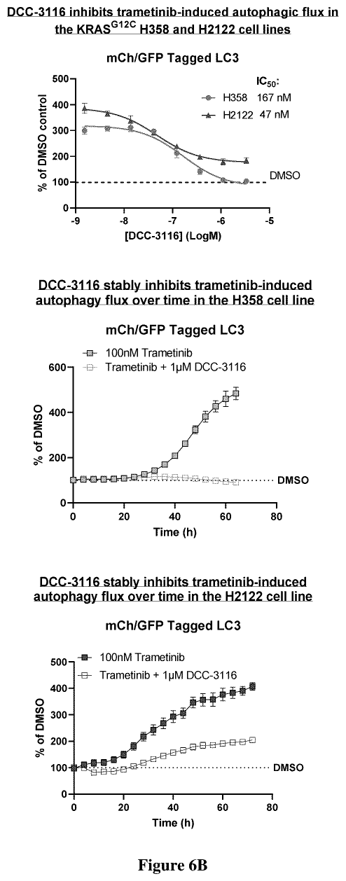

Further, trametinib induces ULK activity and autophagic flux in multiple NSCLC cell lines, which is inhibited by DCC-3116, as shown in FIGS. 6A and 6B.

### Protocol for FIGS. 6A and 6B

### Cell Culture:

H358 (ATCC #CRL-5807), H1373 (ATCC #CRL-5866) and H2122 (ATCC #CRL-5985) cells were grown in RPMI medium (ThermoFisher #21875034) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without a titration of trametinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or trametinib or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

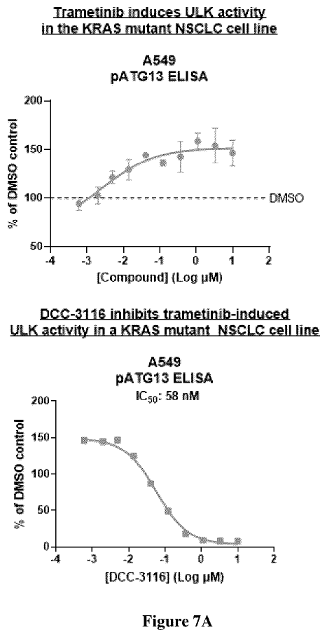

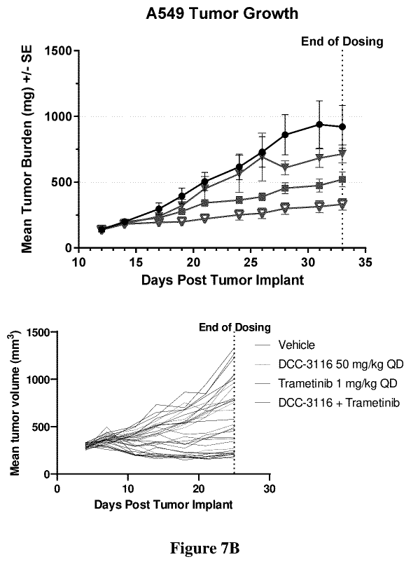

Additionally, trametinib induces ULK activity in the A549* NSCLC Cell Line which is Inhibited by DCC-3116, as shown in FIGS. 7A and 7B.

### Protocol for FIGS. 7A and 7B

### Cell Culture:

A549 (ATCC #CCL-185) cells were grown in DMEM medium (ThermoFisher #11965-084) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without a titration of trametinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

### In Vivo Efficacy Models:

Female SCID mice (NOD.C.B-17/IcrHsd-Prkdc^{scid}); Envigo; 6-7 weeks old) were inoculated subcutaneously in the right high axilla with five million cells in serum-free medium mixed with an equal volume of Matrigel. When tumor burdens reached 140 mm³ on average on day 12, mice were randomly assigned into groups such that the mean tumor burden for all groups was within 10% of the overall mean tumor burden for the study population. Groups were treated on days 12-33 as follows: Vehicle control (0.5% CMC/0.1% Tw80/0.45% NaCl), Trametinib 1 mg/kg PO QD, DCC-3116 50 mg/kg PO BID or the combination of Trametinib 1 mg/kg PO QD+DCC-3116 50 mg/kg PO BID.

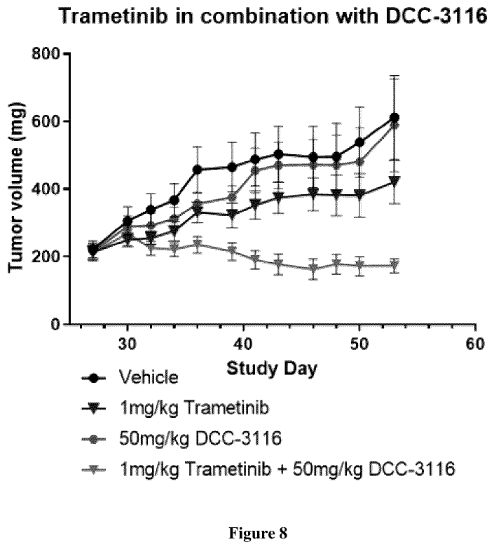

Also, DCC-3116 combines with trametinib in the H358 xenograft to induce tumor regressions, as shown in FIG. 8.

### Protocol for FIG. 8

pATG13 ELISA:

Cells were cultured in assay plates and titrated DCC-3116 was added to each well with or without a titration of trametinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or trametinib or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

### In Vivo Efficacy Models:

Female nude mice (Hsd:Athymic Nude-Foxn1nu; Jackson; 6-8 weeks old) were inoculated subcutaneously in the right high axilla with five million cells in Dulbecco's Phosphate buffered saline mixed with an equal volume of Matrigel. When tumor burdens reached 200-300 mm3 average, mice were randomly assigned into groups such that the mean tumor burden for all groups was within 10% of the overall mean tumor burden for the study population. Groups were treated as follows: Vehicle control (0.5% CMC/0.1% Tw80/0.45% NaCl), 0.5 mg/kg Trametinib alone or with either the 25 mg/kg dose of DCC-3116 or the 50 mg/kg dose of DCC-3116. Trametinib was dosed with a PBS vehicle. Tumor burden was measured twice a week.

### Example 4. Combination Studies of DCC-3116 and Binimetinib

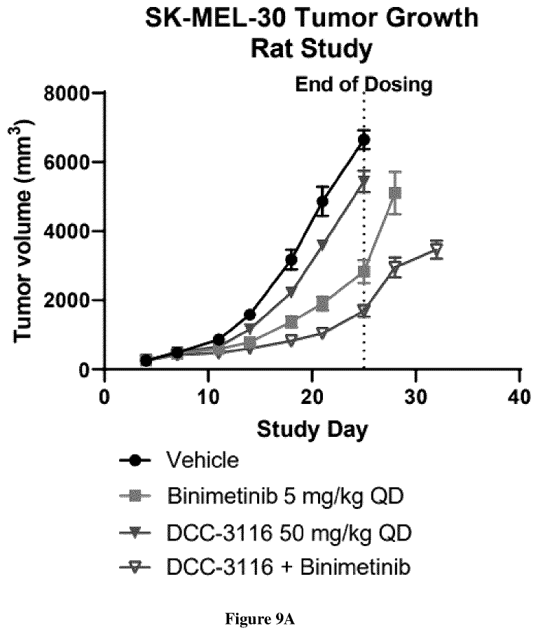

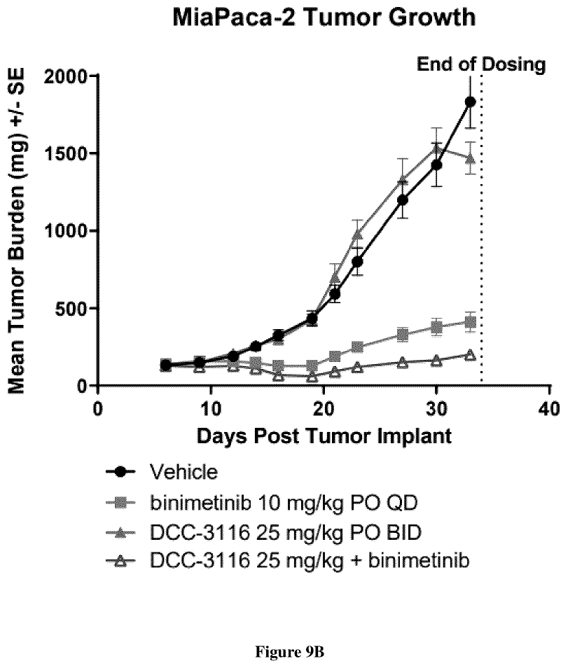

Combinations studies of DCC-3116 and binimetinib were evaluated. For example, DCC-3116 combines with binimetinib in NRAS mutant melanoma and KRAS^{G12C} mutant pancreatic xenograft models, as shown in FIGS. 9A and 9B.

### Protocol for FIGS. 9A and 9B

### In Vivo Efficacy Models:

Female athymic nude rats were inoculated with 5×106 cells/animal from the cell line SK-MEL-30. Animals were inoculated subcutaneously into the right flank at a volume of 0.2 mL cell/matrigel matrix suspension. When a mean tumor volume of 255 mm3 was reached, animals were assigned to 10 groups (n=8 per group) based on tumor measurement and body weight. Animals were administered vehicle control article (Group 01), DCC-3116 (DP-8218)—(Group 02), trametinib (Group 03), binimetinib (Group 04), LY3009120 (Group 05), LY3214996 (Group 06), DCC-3116+trametinib (Group 07), DCC-3116+binimetinib (Group 08), DCC-3116+LY3009120 (Group 09), or DCC-3116+LY3214996 (Group 10) for 21 days. Tumor volume and body weight measurements were recorded for 14 days post the end of the dosing phase. Animals were sacrificed when the tumor burden limit of 5000 mm3 was reached.

Female athymic nude rats were inoculated with 5×106 cells/animal from the cell line MiaPaca-2. Animals were inoculated subcutaneously into the right flank at a volume of 0.2 mL cell/matrigel matrix suspension. When a mean tumor volume of 247 mm³ was reached, animals were assigned to 10 groups (n=8 per group) based on tumor measurement and body weight. Animals were administered vehicle control article, DCC-3116, trametinib, binimetinib, DCC-3116+binimetinib for 21 days. Tumor volume and body weight measurements were recorded for 14 days post the end of the dosing phase. Animals were sacrificed when the tumor burden limit of 5000 mm3 was reached.

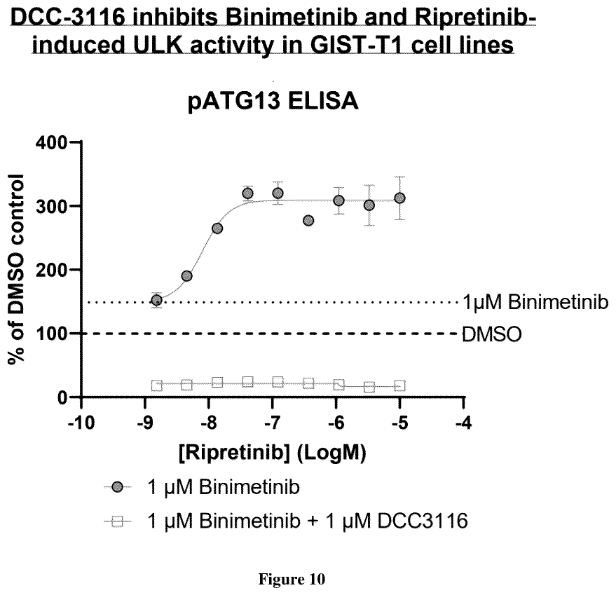

Additionally, it was found that binimetinib induces ULK Activity in the GIST-T1 cell line which is inhibited by DCC-3116, as shown in FIG. 10.

### Protocol for FIG. 10

### Cell Culture:

GIST-T1 cells were grown in DMEM medium (ThermoFisher #11965-084) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without 1 μM binimetinib and/or a titration of ripretinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

### Example 5. Combination Studies of DCC-3116 and Ripretinib

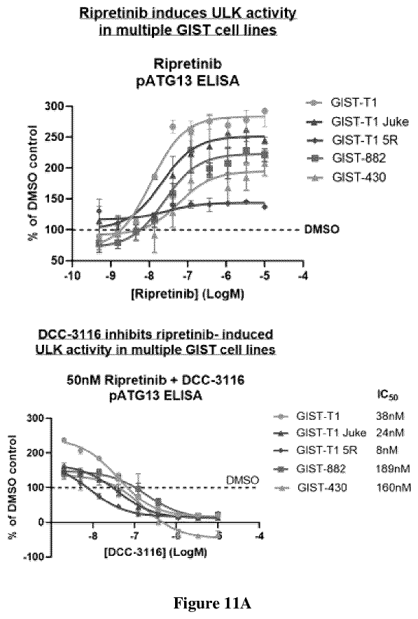

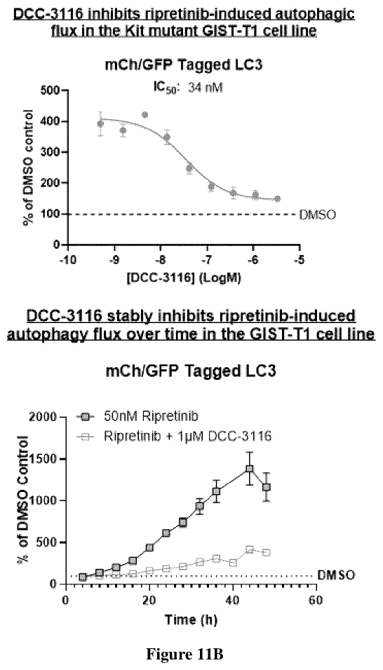

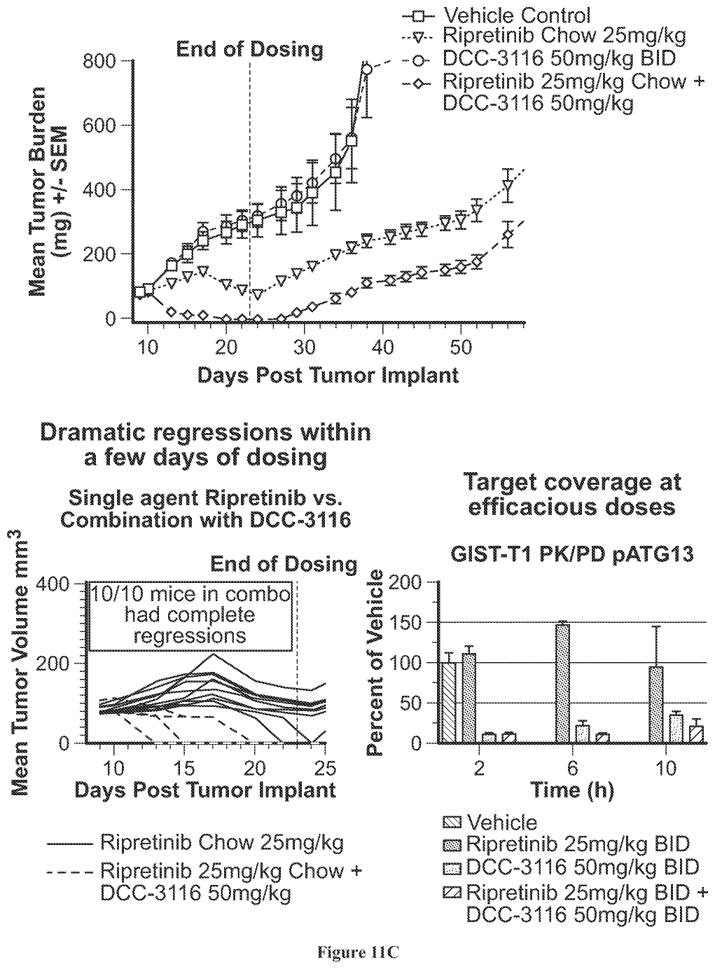

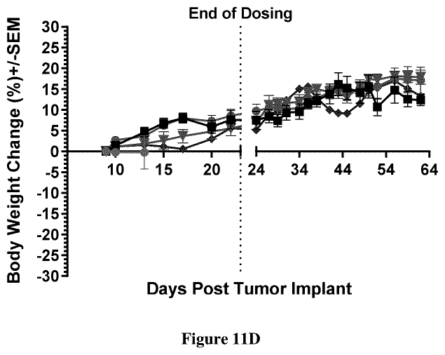

Combination Studies of DCC-3116 and ripretinib were evaluated. For example, DCC-3116 combines with ripretinib in KIT-Mutated gastrointestinal stromal tumors (GIST), as shown in FIGS. 11A, 11B, 11C, and 11D.

### Protocol for FIGS. 11A, 11B, 11C, and 11D

### Cell Culture:

GIST cell lines were grown in DMEM medium (ThermoFisher #11965-084) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without a titration of ripretinib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or ripretinib or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

### In Vivo Efficacy Models:

Female Balb/c nude mice (CAnN.Cg-Foxn1nu/Crl; Vital River Laboratories Research Models and Services (Envigo); 6-7 weeks old) were inoculated subcutaneously in the right high axilla with five million cells in Dulbecco's phosphate buffered saline mixed with an equal volume of Matrigel. When tumor burdens reached 82 mm³ on average, mice were randomly assigned into groups such that the mean tumor burden for all groups was within 10% of the overall mean tumor burden for the study population. Groups were treated on days 9-23 as follows: Vehicle control (0.5% CMC/0.1% Tw80/0.45% NaCl), 25 mg/kg Ripretinib chow, 50 mg/kg DCC-3116, 25 mg/kg Ripretinib chow+50 mg/kg DCC-3116. Tumor burden was measured twice a week. Animals were followed post-dosing to measure tumor regrowth.

### Example 6. Combination Studies of DCC-3116 and KRAS G12C or KRAS G12D Inhibitors

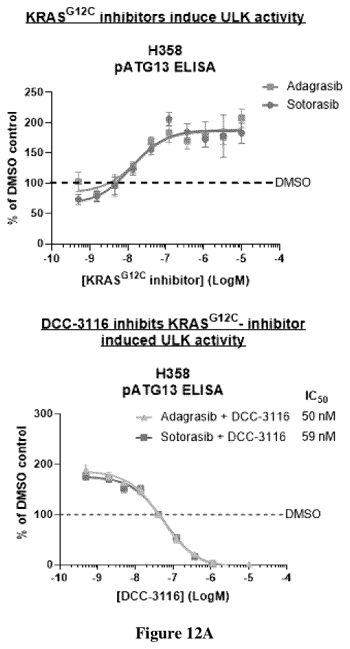

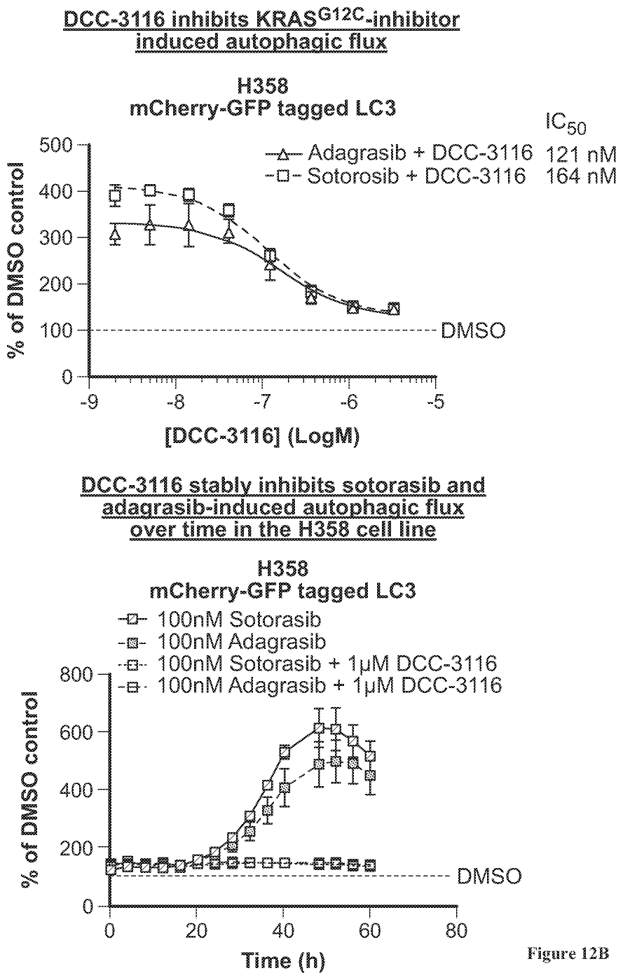

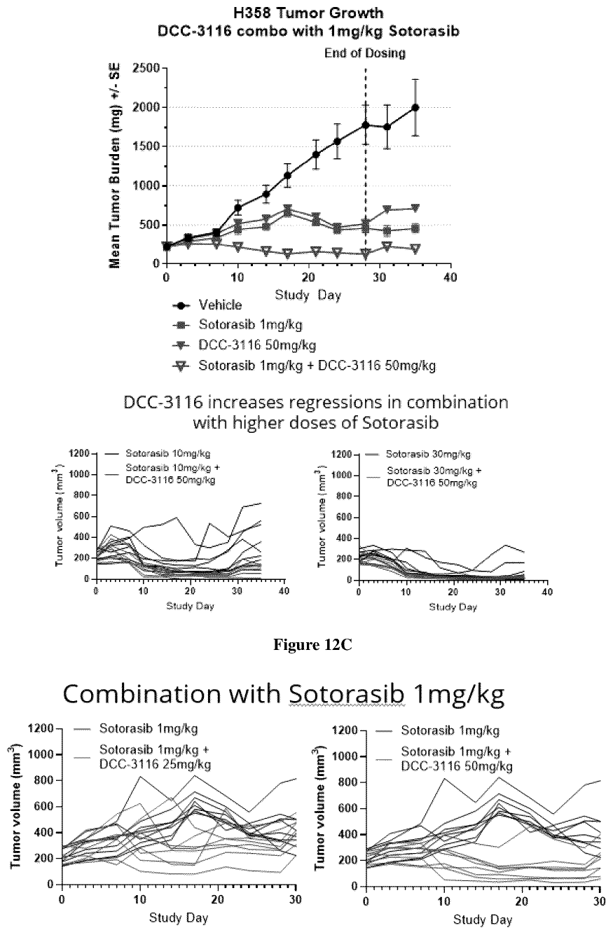

Combination Studies of DCC-3116 and sotorasib were evaluated. For example, DCC-3116 increases tumor regression with sotorasib in KRAS^{G12C}-Mutated NSCLC as shown in FIGS. 12A, 12B, and 12C.

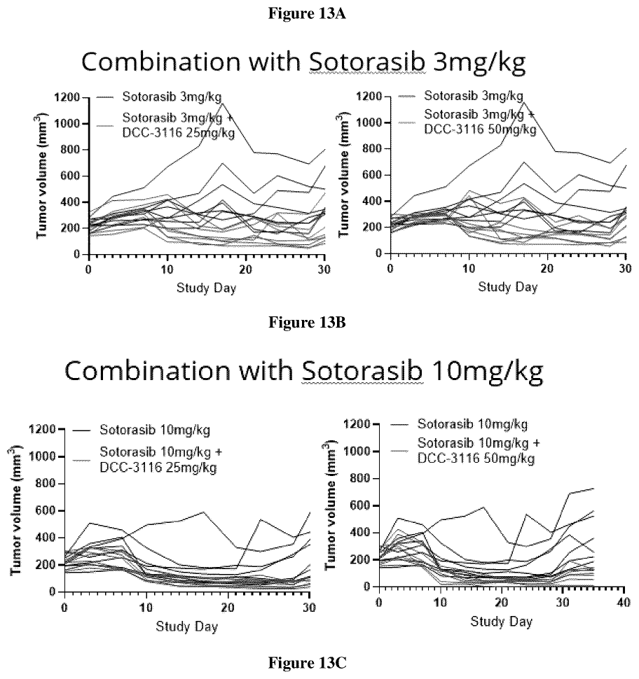

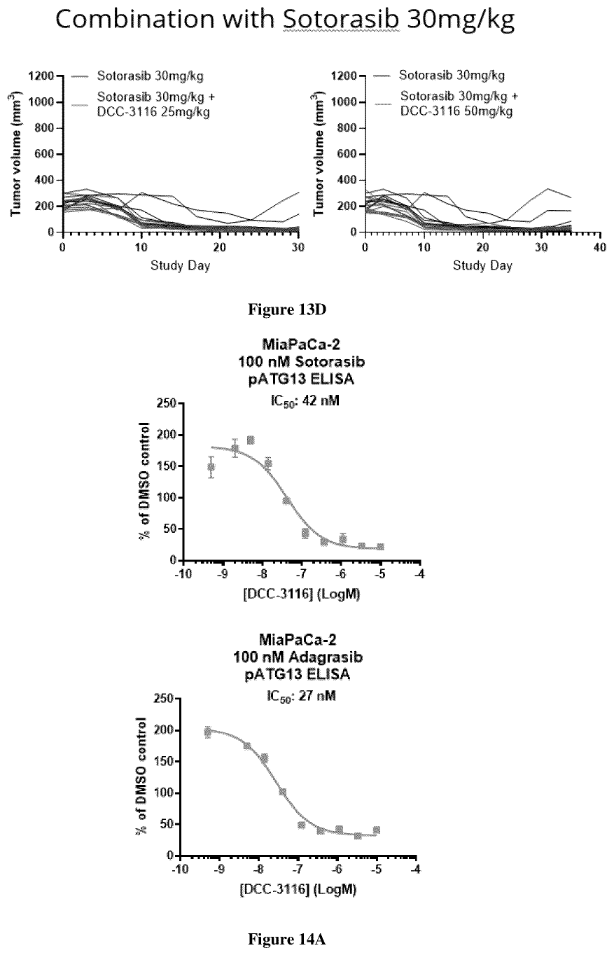

Further, spaghetti plot analysis demonstrates superior tumor growth inhibition and/or regression in all combination cohorts, as shown in FIGS. 13A, 13B, 13C, and 13D (1 mg/kg, 3 mg/kg, 10 mg/kg, and 30 mg/kg, respectively).

### Protocol for FIGS. 12A, 12B, and 12C and FIGS. 13A, 13B, 13C, and 13D

### Cell Culture:

H358 (ATCC #CRL-5807) cells were grown in RPMI medium (ThermoFisher #21875034) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated compound was added to each well with or without titrated adagrasib or sotorasib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or sotorasib or adagrasib or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

### In Vivo Efficacy Models:

Female nude mice (Hsd:Athymic Nude-Foxn1^{nu}; Jackson; 6-8 weeks old) were inoculated subcutaneously in the right high axilla with five million cells in Dulbecco's Phosphate buffered saline mixed with an equal volume of Matrigel. When tumor burdens reached 221 mm³ on average on day 20, mice were randomly assigned into groups such that the mean tumor burden for all groups was within 10% of the overall mean tumor burden for the study population. Groups were treated on days 20-41 as follows: Vehicle control (0.5% CMC/0.1% Tw80/0.45% NaCl), AMG-510 1 mg/kg PO QD, AMG-510 3 mg/kg PO QD, AMG-510 10 mg/kg PO QD, AMG-510 30 mg/kg PO QD, DCC-3116 25 mg/kg PO BID, DCC-3116 50 mg/kg PO BID or the combination of AMG-510 1 mg/kg PO QD, AMG-510 3 mg/kg PO QD, AMG-510 10 mg/kg PO QD, AMG-510 30 mg/kg PO QD with either the 25 mg/kg dose of DCC-3116 or the 50 mg/kg dose of DCC-3116. The vehicle used for AMG-510 was 0.5% Hydroxypropyl Methylcellulose (w/v), 1% polysorbate.

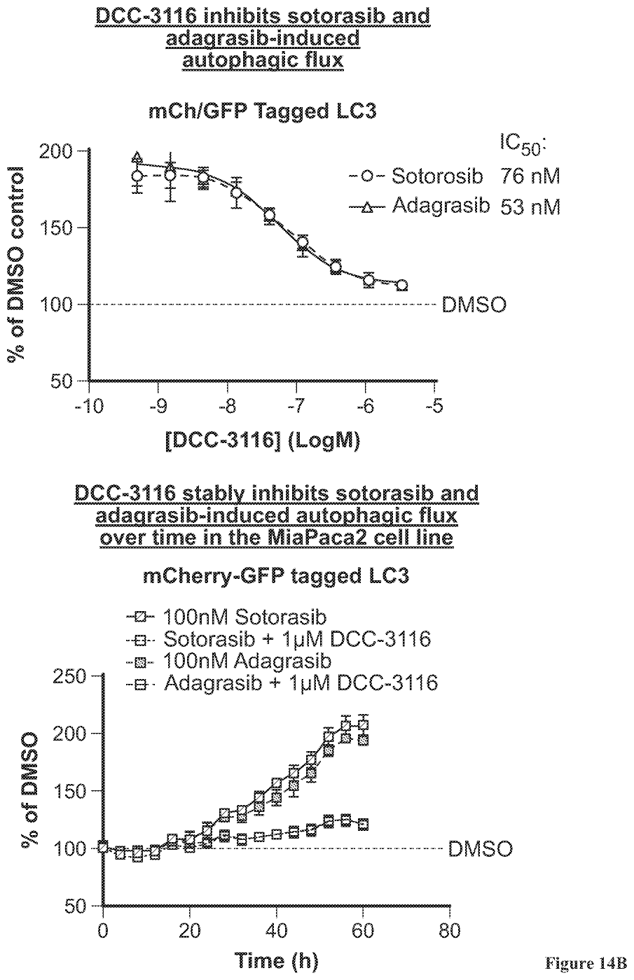

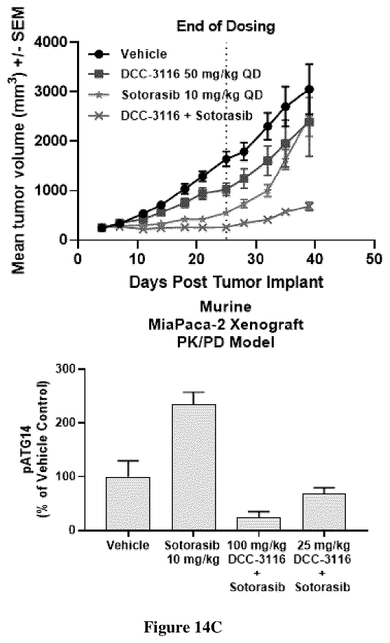

Additionally, DCC-3116 combination with sotorasib was shown to deepen and prolong responses in KRAS^{G12C}-Mutated Pancreatic Cancer, as shown in FIGS. 14A, 14B, and 14C.

### Protocol for FIGS. 14A, 14B, and 14C

### PK/PD Model:

Female nude mice were inoculated subcutaneously in the right high axilla on Day 0 with 1.0×10⁷ live MiaPaca-2 cells per animal. When tumor burdens reached on average 184 mm3 on day 5, mice were randomly assigned into groups. Groups were treated for seven days with oral doses of: Vehicle control BID (n=3); sotorasib 10 mg/kg QD (n=9); DCC-3116 50 mg/kg BID in combination with trametinib 1 mg/kg QD (n=9); or DCC-3116 25 mg/kg BID in combination with sotorasib 10 mg/kg QD (n=9). Three mice/group were euthanized at 2 hrs after the final dose. Tumor samples were powdered over liquid nitrogen and stored at −80° C. before being shipped to Deciphera on dry ice.

The level of phosphorylation at Ser-318 of ATG13 mediated by ULK1/2 kinases in tumor lysates was measured using a sandwich ELISA that captures total ATG13 onto an ELISA plate, followed by binding phosphorylated kinase with an anti-ATG13-S318-phospho-specific antibody conjugated to biotin, and detecting the bound phosphorylated ATG13 with streptavidin-linked horseradish peroxidase (HRP). A substrate solution for HRP was added and the absorbance was measured using a plate reader.

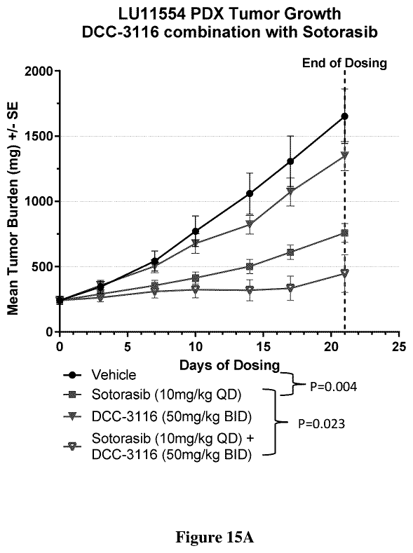

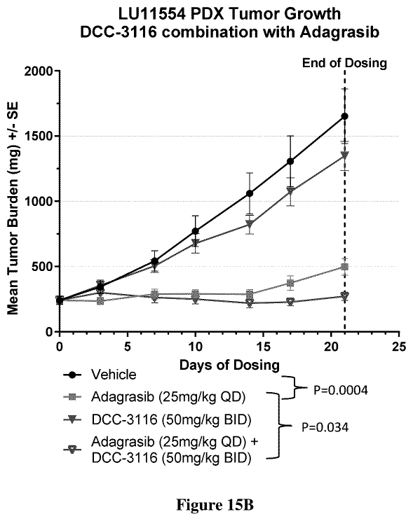

DCC-3116 was also shown to combine with KRAS G12C Inhibitors sotorasib and adagrasib in a PDX Xenograft Lung Cancer Model, as shown in FIG. 15A (sotorasib) and FIG. 15B (adagrasib).

### Protocol for FIGS. 15A and 15B

### In Vivo Efficacy Models:

In the LU11554 PDX model, female NOD/SCID (NOD.C.B-17/IcrHsd-Prkdc^{scid}; Jackson Labs; 8-9 weeks old) were inoculated subcutaneously in the right high axilla with tumor chunks. When tumor burdens reached 190 mm³ on average, mice were randomly assigned into groups such that the mean tumor burden for all groups was within 10% of the overall mean tumor burden for the study population. Groups were treated as follows: Vehicle (0.5% CMC/0.1% Tween 80/0.45% NaCl), 10 mg/kg sotorasib, 25 mg/kg adagrasib, 50 mg/kg DCC-3116, the combination of 10 mg/kg sotorasib+50 mg/kg DCC-3116 or the combination of 25 mg/kg adagrasib+50 mg/kg DCC-3116. Animals were dosed for 21 days with tumor burden measurements taken twice a week.

DCC-3116 was also shown to combine with sotorasib and adagrasib in a KRAS^{G12C}-Mutated colorectal cancer cell line, SW1463, as shown in FIGS. 16A and 16B.

### Protocol for FIGS. 16A and 16B

### Cell Culture:

SW1463 (ATCC #CCL-234) cells were grown in RPMI medium (ThermoFisher #21875034) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without titrated adagrasib or sotorasib. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Further, Compound 1 inhibits ULK activity and autophagic flux induced by KRAS^{G12D} Inhibitor, MRTX-1133, in a human KRAS^{G12D} pancreatic cell line, as shown in FIGS. 17A and 17B.

### Protocol for FIGS. 17A and 17B

pATG13 ELISA:

Cells were cultured in assay plates and titrated compound 1 was added to each well with or without titrated MRTX-1133. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with Compound 1 and/or MRTX-1133 or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

### MiaPaca-2 Xenograft Pharmacokinetic/Pharmacodynamic (PK/PD) Model

The MiaPaca-2 xenograft model was performed in compliance with all the laws, regulations and guidelines of the National Institutes of Health (NIH) and with the approval of the Animal Care and Use Committee of Labcorp (Ann Arbor, MI), an AAALAC accredited facility. Food and water were provided ad libitum. All mice were observed for clinical signs at least once daily. Female Envigo Nude mice (6-7 weeks old) were inoculated subcutaneously just below the right high axilla with ten million cells in Dulbecco's Phosphate Buffered Saline with 50% Matrigel, using a 27-gauge needle and syringe. When tumor burdens reached 182 mm³ on average on day 6, mice were randomly assigned into groups such that the mean tumor burden for all groups was within 10% of the overall mean tumor burden for the study population. Groups were treated on days 6-13 as follows: vehicle control (0.5% CMC/0.1% Tween 80/0.45% NaCl) (dosed PO) (n=3); sotorasib 10 mg/kg PO QD (n=9); DCC-3116 100 mg/kg PO BID (n=9); DCC-3116 25 mg/kg PO BID (n=9); and DCC-3116 10 mg/kg PO BID (n=9). At 2 h post final dose in the vehicle group, and 2, 6, and 10 h post final dose in the treatment groups, 3 mice from each group were sacrificed for tumor and plasma sampling. Blood samples were collected in K2EDTA tubes and processed into plasma, snap frozen in liquid nitrogen then stored at −80° C. Plasma samples were subjected to pharmacokinetic analysis using liquid chromatography coupled with tandem mass spectrometry analysis (Cayman Chemical, Ann Arbor, MI). Tumors were harvested and powdered over liquid nitrogen in covaris bags and stored at −80° C.

Pharmacokinetic analysis revealed that when sotorasib was dosed as a single agent, it led to an average Cₘₐₓ of 296 ng/mL sotorasib in the plasma, with an average AUC_{0-10 h} of 963 ng*h/mL. Sotorasib dosed in combination with 100 mg/kg DCC-3116 led to an average Cₘax of 74 ng/mL sotorasib in the plasma, with an average AUC_{0-10 h} of 385 ng*h/mL. Sotorasib dosed in combination with 25 mg/kg DCC-3116 led to an average Cₘax of 170 ng/mL sotorasib in the plasma, with an average AUC_{0-10 h} of 727 ng*h/mL. Sotorasib dosed in combination with 10 mg/kg DCC-3116 led to an average Cₘax of 76 ng/mL sotorasib in the plasma, with an average AUC_{0-10 h} of 344 ng*h/mL. The differences in results for Cₘₐₓ and AUC_{0-10 h} were not statistically significant amongst groups.

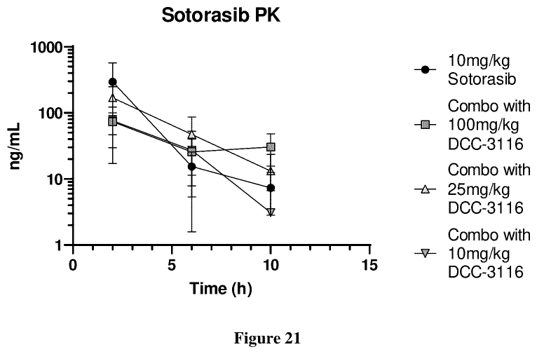

PK results of sotorasib in combination with DCC-3116 in a MiaPaca-2 PK/PD model are shown in FIG. 21. Results are also provided for various doses in Table 2 below:

TABLE 2

| | Sotorasib | Sotorasib |
|---|---|---|
| | Cmax | AUC 0-10 |
| Dosing | (ng/mL) | (h*ng/mL) |
| 10 mg/kg sotorasib | 296 | 963 |
| 10 mg/kg sotorasib + 100 mg/kg | 74 | 385 |
| DCC-3116 | | |
| 10 mg/kg sotorasib + 25 mg/kg | 170 | 727 |
| DCC-3116 | | |
| 10 mg/kg sotorasib + 10 mg/kg | 76 | 344 |
| DCC-3116 | | |

Nude Mouse PK Study with Adagrasib and DCC-3116 Treatment

The nude mouse PK study was performed at Attentive Science (Stilwell, KS), an AAALAC accredited facility. Food and water were provided ad libitum. All mice were observed for clinical signs at least once daily. Female athymic nude mice (n=3 per group) were dosed for 5 consecutive days. Groups were treated as follows: DCC-3116 25 mg/kg PO BID in a vehicle of 0.5% CMC/0.1% Tween 80/0.5% NaCl; DCC-3116 50 mg/kg PO BID in a vehicle of 0.5% CMC/0.1% Tween 80/0.5% NaCl; adagrasib 100 mg/kg PO QD in a vehicle of 10% Captisol, 50 mM citrate buffer, pH 5.0; DCC-3116 25 mg/kg PO BID+adagrasib 100 mg/kg PO QD; and DCC-3116 50 mg/kg PO BID+adagrasib 100 mg/kg PO QD. At 0, 0.5, and 2 h post final dose blood samples were collected from mice from each group. Blood samples were collected in K2EDTA tubes and processed into plasma, then stored at −20° C. Plasma samples were subjected to pharmacokinetic analysis using liquid chromatography coupled with tandem mass spectrometry analysis.

Pharmacokinetic analysis revealed that when adagrasib was dosed as a single agent, it led to an average Cₘₐₓ of 2,530 ng/mL adagrasib in the plasma, with an average AUC_{0-2 h} of 4,230 ng*h/mL. When adagrasib was dosed in combination with 25 mg/kg DCC-3116, it led to an average Cₘₐₓ of 1,880 ng/mL adagrasib in the plasma, with an average AUC_{0-2 h} of 2,970 ng*h/mL. When adagrasib was dosed in combination with 25 mg/kg DCC-3116, it led to an average Cₘax of 2,060 ng/mL adagrasib in the plasma, with an average AUC_{0-2 h} of 3,560 ng*h/mL. The differences in results for Cₘₐₓ and AUC_{0-2 h} were not statistically significant amongst groups. When dosed as a single agent at 25 mg/kg, DCC-3116 treatment led to average Cₘax of 749 ng/mL DCC-3116 in the plasma, with an average AUC_{0-2 h} of 876 ng*h/mL. When dosed as a single agent at 50 mg/kg, DCC-3116 treatment led to average Cₘₐₓ of 1,380 ng/mL DCC-3116 in the plasma, with an average AUC_{0-2 h} of 1,750 ng*h/mL. When 25 mg/kg DCC-3116 was dosed in combination with adagraisb, treatment led to average Cₘₐₓ of 937 ng/mL DCC-3116 in the plasma, with an average AUC_{0-2 h} of 1,150 ng*h/mL. When 50 mg/kg DCC-3116 was dosed in combination with adagraisb, treatment led to average Cₘₐₓ of 1,210 ng/mL DCC-3116 in the plasma, with an average AUC_{0-2 h} of 1,650 ng*h/mL. The differences in results for Cₘₐₓ and AUC_{0-2 h} were not statistically significant amongst groups.

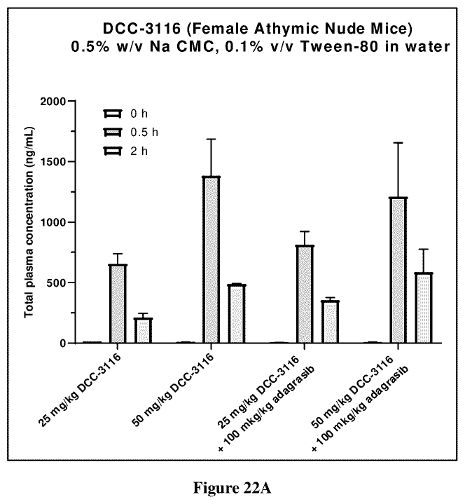

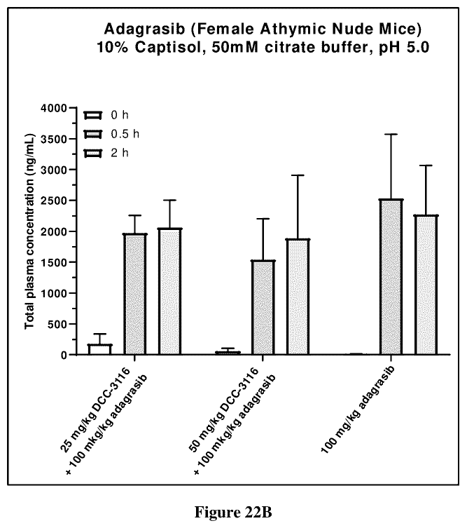

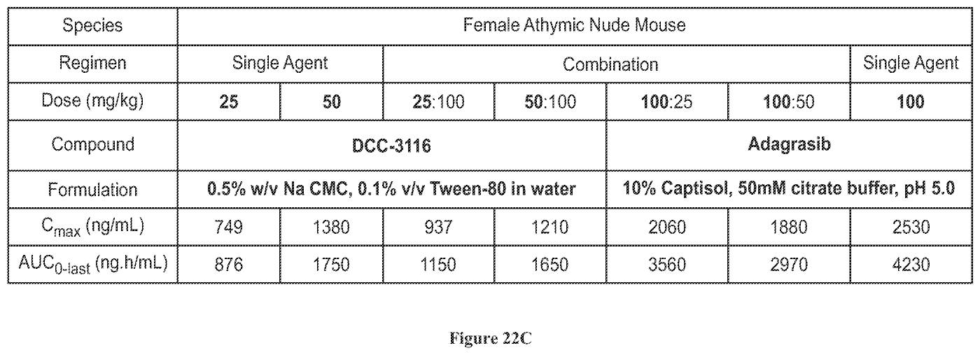

Plasma concentration results of DCC-3116 in combination with adagrasib in female athymic nude mice are shown in FIG. 22A (0.5% w/v Na CMC, 0.1% v/v Tween®-80 in water) and FIG. 22B (10% Captisol®, 50 mM citrate buffer, pH 5.0).

PK results of adagrasib in combination with DCC-3116 in nude mice after 5 days of dosing are shown in FIG. 22C.

### Example 7. Combination Studies of DCC-3116 and RAF Inhibitors

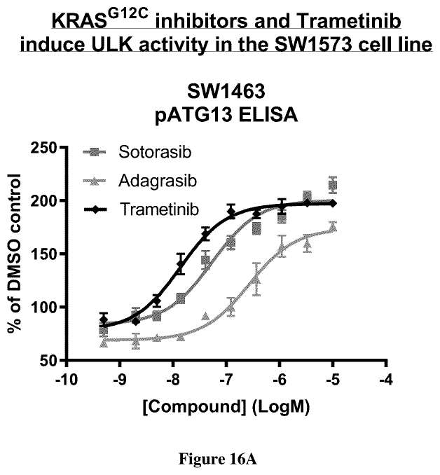

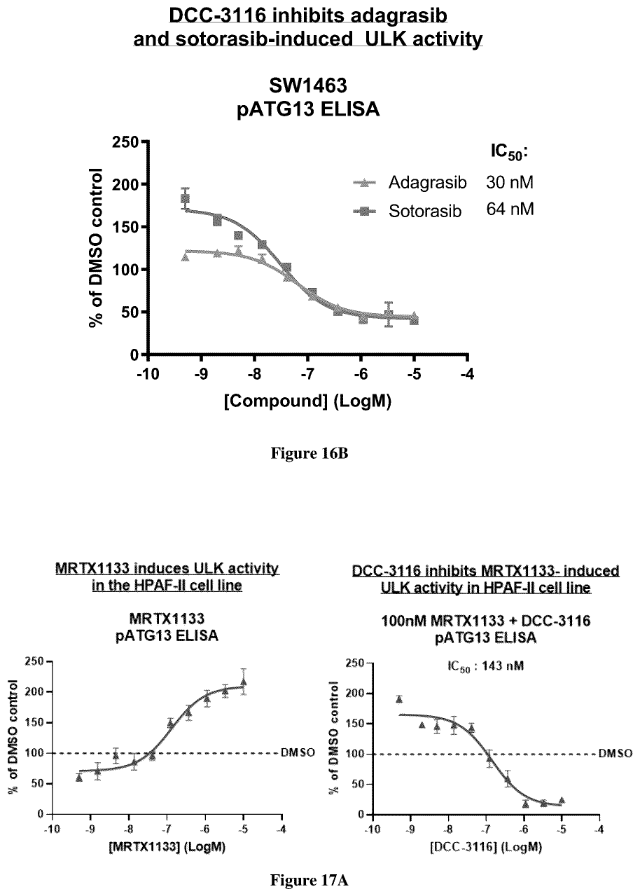

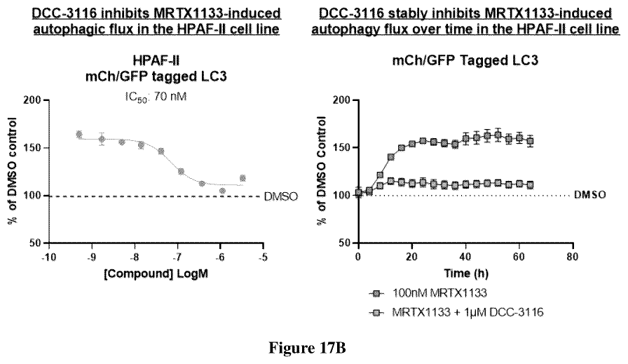

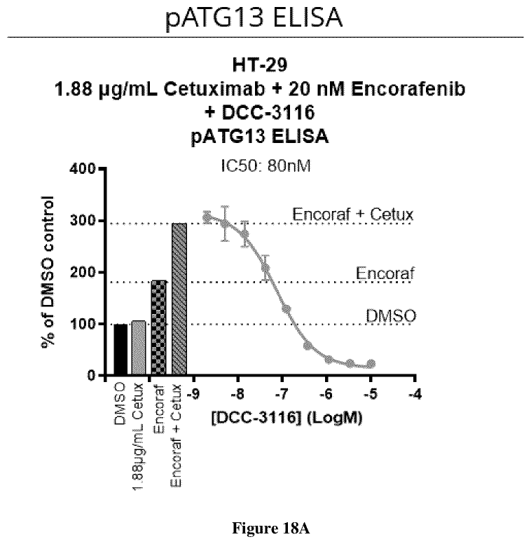

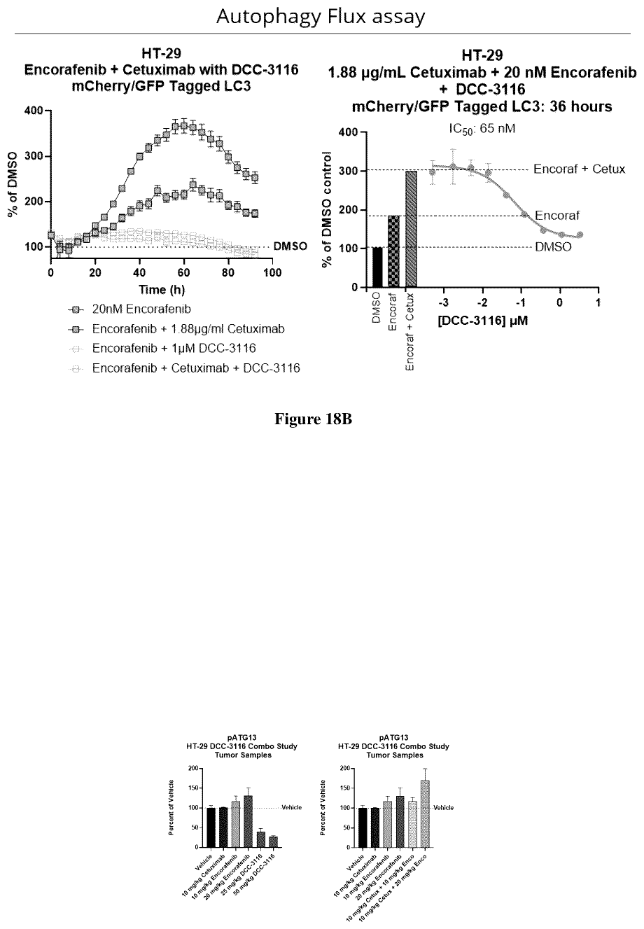

Combination Studies of DCC-3116 and RAF inhibitors were evaluated. For example, DCC-3116 was shown to combine with encorafenib and cetuximab in a BRAF^{V600E}-mutated colorectal cancer cell line, as shown in FIGS. 18A and 18B.

### Protocol for FIGS. 18A and 18B

### Cell Culture:

HT29 cells were grown in McCoy's 5A (ThermoFisher #1660082) containing 10% FBS (ThermoFisher #16000-044 or equivalent) and 1% penicillin-streptomycin-L-Glutamine (Life technologies, 10378-016).

pATG13 ELISA:

Cells were cultured in assay plates and titrated DCC-3116 was added to each well with or without titrated encorafenib and 1.88 g/mL of cetuximab. Plates were incubated for 16 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated kinase with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For each cell line, 10 ml of appropriate media was mixed with Polybrene (VectorBuilder, Cat #PL0001, stock concentration 5 mg/ml, 200 ul) to a final concentration of 15 ng/μL. These solutions were added to the 6-well plate with various cell lines plated the previous day. Cells were checked after 22 hours of incubation. Media was then removed from the cells and incubated with fresh media containing puromycin (Sigma Cat #P9620). Cells that survived puromycin selection and successfully expressed mCherry-GFP-LC3 were used for flux assays.

### Flux Assays:

Cells are seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells are treated with DCC-3116 and/or encorafenib and cetuximab or DMSO as a control. Cells are placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μM2/images) of red (mCherry) divided by (GCUX μM2/images) green (GFP) to measure the ratio of red/green signal change over time.

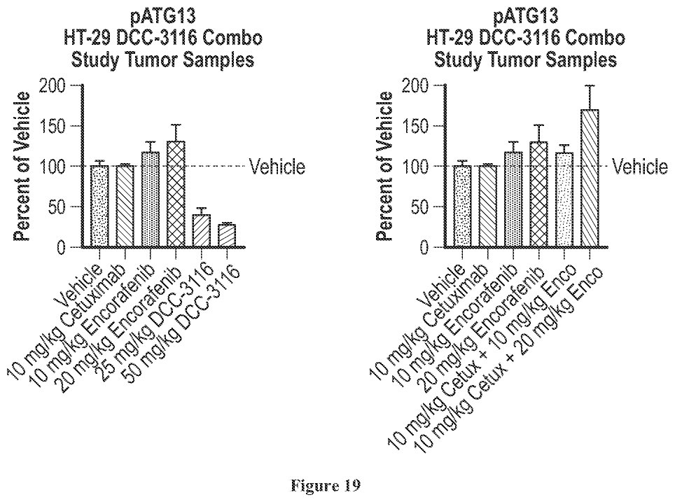

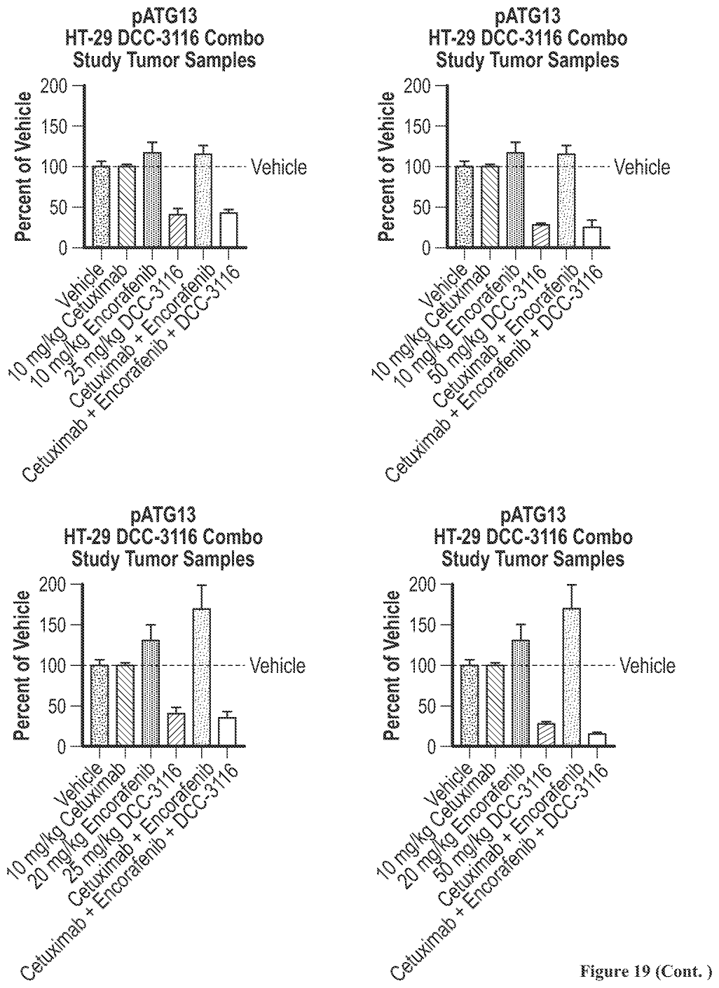

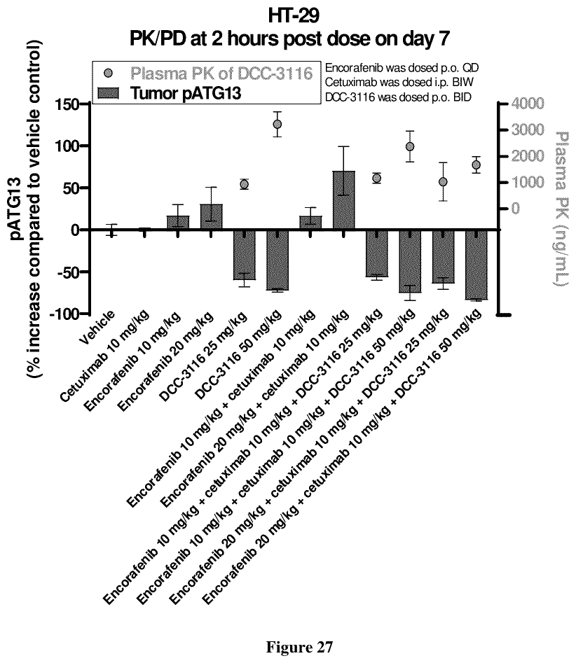

Further, DCC-3116 inhibits encorafenib and cetuximab induced pATG13 in HT-29 tumors, as shown in FIG. 19. PK/PD studies of DCC-3116 combined with encorafenib and cetuximab at 2 hours post-dosing on day 7 is shown in FIG. 27.

### Protocol for FIG. 19 and FIG. 27

### PK/PD Models:

Female NOD/SCID (NOD.C.B-17/IcrHsd-Prkdc^{scid}; Jackson Labs; 8-9 weeks old) were inoculated subcutaneously in the right high axilla on Day 0 with 3.0×10⁶ live HT-29 cells per animal. When tumor burdens reached on average 247 mm³, mice were randomly assigned into groups. Groups were treated for seven days with oral doses of: Vehicle control BID (n=3); Cetuximab 10 mg/kg BIW (n=3); Encorafenib 10 mg/kg QD, Encorafenib 20 mg/kg QD, DCC-3116 25 mg/kg BID, DCC-3116 50 mg/kg BID, 50 mg/kg DCC-3116 BID in combination with Cetuximab 10 mg/kg+Encorafenib 10 mg/kg (n=3); or DCC-3116 25 mg/kg BID in combination with Cetuximab 10 mg/kg+Encorafenib 10 mg/kg 1 mg/kg QD (n=3). 50 mg/kg DCC-3116 BID in combination with Cetuximab 10 mg/kg+Encorafenib 20 mg/kg (n=3); or DCC-3116 25 mg/kg BID in combination with Cetuximab 10 mg/kg+Encorafenib 20 mg/kg 1 mg/kg QD (n=3). Three mice/group were euthanized at 2 hrs after the final dose. Plasma samples were collected and snap frozen for pharmacokinetic analysis. Tumor samples were powdered over liquid nitrogen and stored at −80° C.

The level of phosphorylation at Ser-318 of ATG13 mediated by ULK1/2 kinases in tumor lysates was measured using a sandwich ELISA that captures total ATG13 onto an ELISA plate, followed by binding phosphorylated kinase with an anti-ATG13-S318-phospho-specific antibody conjugated to biotin, and detecting the bound phosphorylated ATG13 with streptavidin-linked horseradish peroxidase (HRP). A substrate solution for HRP was added and the absorbance was measured using a plate reader.

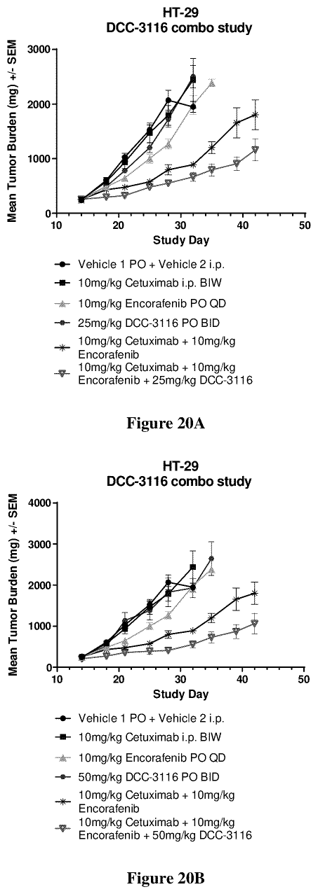

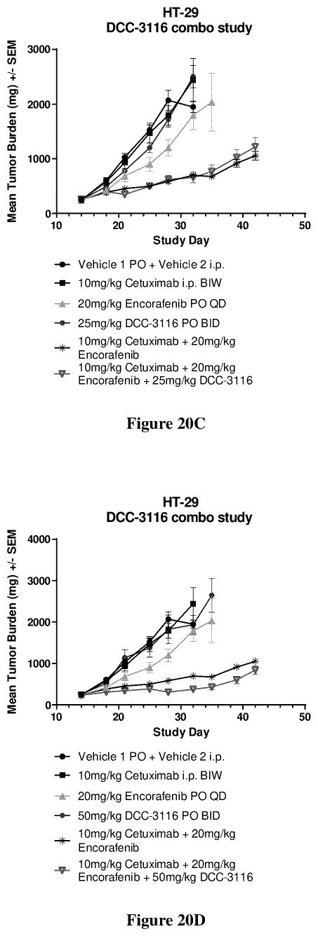

DCC-3116 reduces tumor burden in combination with encorafenib and cetuximab in the BRAF V600E mutated colorectal model, HT-29, as shown in FIGS. 20A, 20B, 20C, and 20D.

### Protocol for FIGS. 20A, 20B, 20C, and 20D

Female NOD/SCID (NOD.C.B-17/IcrHsd-Prkdc^{scid}; Jackson Labs; 8-9 weeks old) were inoculated subcutaneously in the right high axilla on Day 0 with 3.0×10⁶ live HT-29 cells per animal. When tumor burdens reached on average 247 mm³, mice were randomly assigned into groups. Groups were treated for seven days with oral doses of: Vehicle control BID (n=3); Cetuximab 10 mg/kg BIW (n=3); Encorafenib 10 mg/kg QD, Encorafenib 20 mg/kg QD, DCC-3116 25 mg/kg BID, DCC-3116 50 mg/kg BID, 50 mg/kg DCC-3116 BID in combination with Cetuximab 10 mg/kg+Encorafenib 10 mg/kg (n=3); or DCC-3116 25 mg/kg BID in combination with Cetuximab 10 mg/kg+Encorafenib 10 mg/kg 1 mg/kg QD (n=3). 50 mg/kg DCC-3116 BID in combination with Cetuximab 10 mg/kg+Encorafenib 20 mg/kg (n=3); or DCC-3116 25 mg/kg BID in combination with Cetuximab 10 mg/kg+Encorafenib 20 mg/kg 1 mg/kg QD (n=3). Tumor burden was measured twice a week.

### Example 8. Combination Studies of DCC-3116 with RAF and EGFR Inhibitors

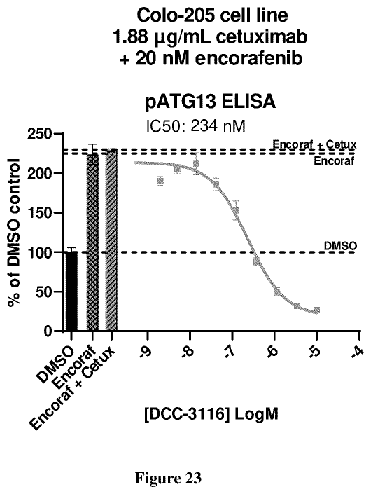

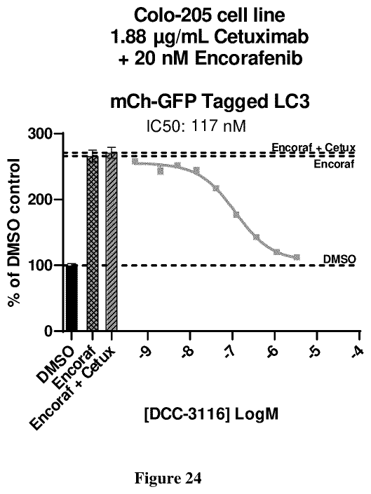

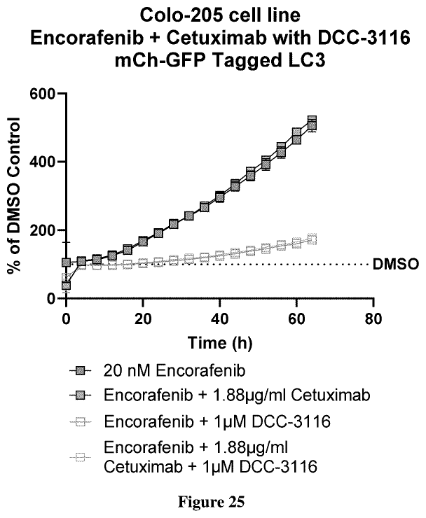

Combination Studies of DCC-3116 and RAF inhibitors were evaluated. For example, DCC-3116 was shown to combine with encorafenib and cetuximab in a BRAFV600E-mutated colorectal cancer cell line, as shown in FIGS. 23-25. DCC-3116 inhibited encorafenib- and cetuximab-induced phosphorylation of ATG13 (FIG. 23). DCC-3116 inhibited encorafenib- and cetuximab-induced autophagic flux (FIGS. 24 and 25).

### Protocol for FIGS. 23-25

### Cell Culture:

Colo-205 cells were grown in RPMI1640 (ThermoFisher #11875-119) containing 10% FBS (ThermoFisher #16000-044 or equivalent) and 1% penicillin-streptomycin-L-Glutamine (Life technologies #10378-016).

pATG13 ELISA:

Cells were cultured in assay plates and titrated DCC-3116 in DMSO was added to each well with or without titrated encorafenib in DMSO and 1.88 g/mL of cetuximab in PBS. Plates were incubated for 45 hr. The level of phosphorylation at Ser-318 of ATG13 (Rockland Immunochemicals #600-401-C49) in cellular lysates was measured using a sandwich ELISA that captures total ATG13 (Cell Signaling #13273) onto an ELISA plate (Costar #2592 or equivalent), followed by binding phosphorylated ATG13 (pATG13) with an anti-ATG13-S318-phospho-specific antibody conjugated to biotin, and detecting the bound phosphorylated ATG13 with streptavidin-linked horseradish peroxidase (HRP) (ThermoFisher #21140). A substrate solution for HRP was added and the absorbance was measured using a plate reader. Data was plotted in GraphPad Prism and fit to a sigmoidal dose response curve to generate IC₅₀ values. Anti-ATG13-S318-phospho-specific antibody was conjugated to biotin using the Biotin Labeling Kit-NH2 (Dojindo Molecular Tech. #LK03-10).

Generation of mCherry/GFP LC3 Cell Lines:

Constructs were designed through VectorBuilder. For the Colo-205 cell line, 10 mL of appropriate media was mixed with Polybrene (VectorBuilder, Cat #PL0001, stock concentration 5 mg/mL, 200 μL) to a final concentration of 15 ng/μL. These solutions were added to the 6-well plate with cells plated the previous day. Cells were checked after 22 hours of incubation. Media was then removed from the cells and cells were incubated with fresh media containing puromycin (Sigma Cat #P9620). Cells that survived puromycin selection and successfully expressed mCherry-GFP-LC3 were used for flux assays.

### Flux Assays:

Cells were seeded in FluoroBrite DMEM (Gibco Ref #A18967-01) supplemented with 10% FBS (Gibco ref #A384000-001) and Pen/Strep/Glutamine (Gibco Ref #10378-016) at appropriate seeding numbers defined empirically. The next day, cells were treated with DCC-3116 and/or encorafenib and cetuximab or DMSO as a control. Cells were placed in the IncuCyte for live cell imaging over the indicated time length at 37° C. with 20% 02 and 5% CO₂. Cells were imaged at 4× objective lens every four hours. The data was analyzed using the Incucyte software by measuring Total Integrated Intensity (GCUX μm²/images) of read (mCherry) divided by (GCUX μm²/images) of green (GFP) to measure the ratio of red/green signal change over time.

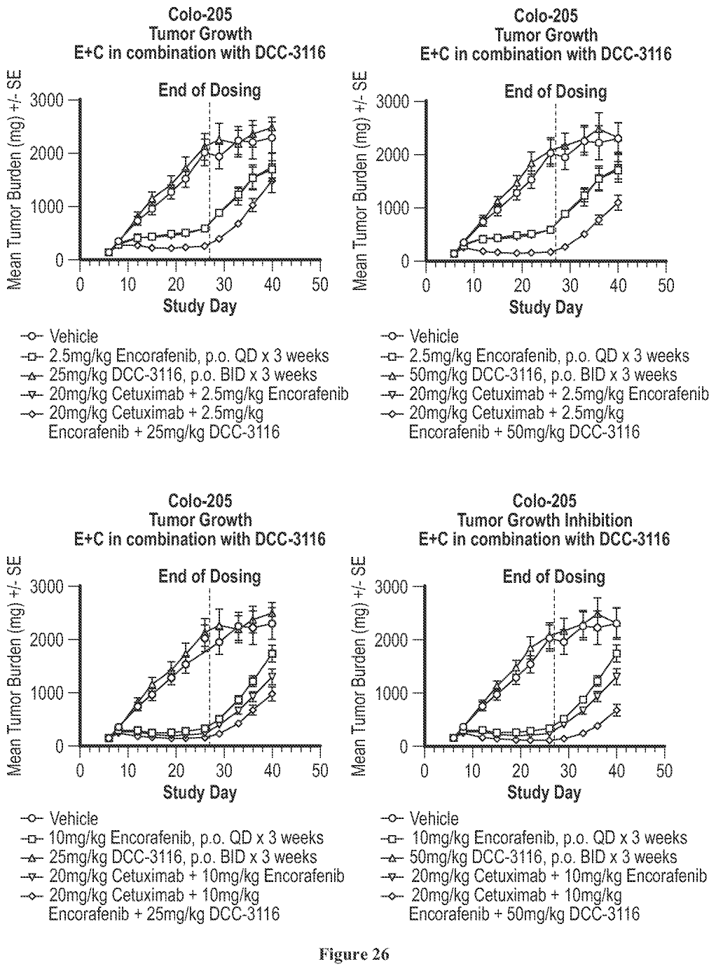

DCC-3116 reduced tumor burden in combination with encorafenib and cetuximab in the BRAF V600E mutated colorectal model, Colo-205, as shown in FIG. 26.

### In Vivo Efficacy Models:

Female BALB/c nude mice (GemPharmatech Co., Ltd; 6-7 weeks old) were inoculated subcutaneously in the right upper flank on Day 0 with 5×10⁶ Colo-205 cells in Dulbecco's Phosphate buffered saline mixed with an equal volume of Matrigel. When tumor burdens reached ˜140 mm³ on average on day 6, mice were randomly assigned into groups (12 study groups, 10 mice per group), such that the mean tumor for all groups was within 10% of the overall mean tumor burden for the study population. Groups were treated on days 6-27 as follows: Vehicle control (0.5% CMC/0.1% Tw80/0.45% NaCl) PO, BID+PBS IP BIW; cetuximab in PBS, 20 mg/kg IP BIW; encorafenib, 2.5 mg/kg PO QD; encorafenib, 10 mg/kg PO QD, DCC-3116 25 mg/kg PO BID; DCC-3116 50 mg/kg PO BID; or the combination of cetuximab+encorafenib 2.5 mg/kg; cetuximab+encorafenib 10 mg/kg; cetuximab+encorafenib 2.5 mg/kg+DCC-3116 25 mg/kg; cetuximab+encorafenib 2.5 mg/kg+DCC-3116 50 mg/kg; cetuximab+encorafenib 10 mg/kg+DCC-3116 25 mg/kg; or cetuximab+encorafenib 10 mg/kg+DCC-3116 50 mg/kg. The vehicle used for cetuximab was PBS. The vehicle used for encorafenib was 0.5% CMC/0.5% Tw80.

### Example 9. A Study of DCC-3116 in Combination with Anticancer Therapies in Participants with Advanced Malignancies

This is a Phase 1/2, multicenter, open-label (unless otherwise specified in a combination-specific module) study of DCC-3116 in combination with anticancer therapies. Modules within the master protocol are defined according to different combinations of DCC-3116 with other anticancer agents.

Experimental Arms and Interventions for the study are described in Table 3 below:

TABLE 3

| Arms | Assigned Interventions |
|---|---|
| Experimental: Dose Escalation (Part 1, Arm | Drug: DCC-3116 |
| A) | Oral Tablet Formulation |
| DCC-3116 tablets in escalating dose | Drug: Cetuximab |
| cohorts in 28-day cycles will be | Solution for Injection |
| administered in combination with | Drug: Encorafenib |
| encorafenib once daily (QD) and | 75 mg capsules |
| cetuximab once every 2 weeks (Q2W). | |
| Experimental: Expansion (Part 2, Arm A) | Drug: DCC-3116 |
| DCC-3116 tablets will be administered in | Oral Tablet Formulation |
| combination with encorafenib and | Drug: Cetuximab |
| cetuximab in 28-day cycles to evaluate | Solution for Injection |
| preliminary efficacy in participants with | Drug: Encorafenib |
| 2nd- or 3rd-line BRAF V600E mutated | 75 mg capsules |
| colorectal cancer (CRC). | |
| Experimental: Dose Escalation (Part 1, Arm | Drug: DCC-3116 |
| B) | Oral Tablet Formulation |
| DCC-3116 tablets in escalating dose | Drug: Ripretinib |
| cohorts in 28-day cycles will be | 50 mg tablets |
| administered in combination with | |
| ripretinib once daily (QD). | |
| Experimental: Expansion (Part 2, Arm B) | Drug: DCC-3116 |
| DCC-3116 tablets will be administered in | Oral Tablet Formulation |
| combination with ripretinib in 28-day | Drug: Ripretinib |
| cycles to evaluate preliminary efficacy in | 50 mg tablets |
| participants with 2nd-line advanced | |
| gastrointestinal stromal tumor (GIST). | |

Outcome measures include the following: Primary Outcome Measures: (i) Incidence of Adverse Events (Escalation Phase): Identify the observed adverse events and serious adverse events associated with DCC-3116 in combination with cetuximab and encorafenib or ripretinib; (ii) Recommended Phase 2 Doses (RP2D) (Escalation Phase): Identify the dose-limiting toxicities for each dose level tested and determine the recommended Phase 2 doses of DCC-3116 in combination with cetuximab and encorafenib or ripretinib; (iii) Objective response rate (ORR) (Expansion Phase): Proportion of participants who achieve CR or PR per RECIST (or mRECIST, as applicable) v1.1.

Secondary Outcome Measures: (i) Duration of response (DoR) (Escalation Phase): DoR is defined as the time interval from the time that the measurement criteria are first met for CR or PR (whichever is first recorded) until the first date that the progressive disease is objectively documented or death, whichever occurs first; (ii) Disease Control Rate (DCR) (Escalation Phase): The DCR is defined as the proportion of participants who achieve CR, PR, or stable disease [SD] at the specified time point per RECIST v1.1; (iii) Time to response (Escalation Phase): Time to response is defined as the time from initiation of treatment until the first assessment demonstrating CR or PR per RECIST (or mRECIST, as applicable) v1.1.; (iv) Progression-free survival (PFS) (Escalation Phase): PFS is defined as the time from initiation of treatment until documented disease progression per RECIST (or mRECIST, as applicable) v1.1 or death, whichever occurs first; (v) Overall Survival (OS): OS is defined as the time from initiation of treatment until death; (vi) Maximum observed concentration (Cₘₐₓ): Measure the maximum observed concentration of DCC-3116 combinations; (vii) Time to maximum observed concentration (Tmax): Measure the time to maximum plasma concentration of DCC-3116 combinations; (viii) Minimum observed concentration (Cmin): Measure the minimum observed concentration of DCC-3116 combinations; (ix) Area under the concentration-time curve (AUC): Measure the AUC of DCC-3116 combinations.

1. A method of treating cancer in a patient in need thereof, comprising:
(i) administering to the patient about 20 mg to about 600 mg, once or twice daily, of a compound represented by Formula (I): 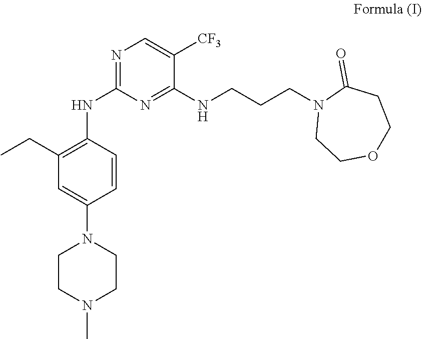 [SMILES: CCc1cc(N2CCN(C)CC2)ccc1Nc1ncc(C(F)(F)F)c(NCCCN2CCOCCC2=O)n1]
or a pharmaceutically acceptable salt thereof; and
(ii) administering to the patient a therapeutically effective amount of one or more additional therapeutic agents.

2. The method of claim 1, comprising administering to the patient about 20 mg to about 400 mg, twice daily, of the compound or pharmaceutically acceptable salt thereof.

3. The method of claim 1, comprising administering to the patient about 50 mg to about 300 mg, twice daily, of the compound or pharmaceutically acceptable salt thereof.

4. The method of claim 1, wherein the one or more additional therapeutic agents is selected from the group consisting of a MAPKAP pathway inhibitor, an EGFR inhibitor, a KIT inhibitor, and a combination thereof.

5. The method of claim 4, wherein the MAPKAP pathway inhibitor is selected from the group consisting of a MEK inhibitor, an ERK inhibitor, a RAF inhibitor, a Ras inhibitor, and a combination thereof.

6-10. (canceled)

11. The method of claim 1, wherein the one or more additional therapeutic agents is selected from the group consisting of trametinib, binimetinib, sotorasib, adagrasib, cetuximab, encorafenib, ripretinib, and a combination thereof.

12. The method of claim 1, wherein the cancer is selected from the group consisting of pancreatic ductal adenocarcinoma, non-small cell lung cancer, colorectal cancer, melanoma, and gastrointestinal stromal tumors.

13-34. (canceled)

35. A method of treating cancer in a patient in need thereof, comprising administering to the patient:
(i) orally administering to the patient about 20 mg to about 600 mg, once or twice daily, of a compound represented by Formula (I): 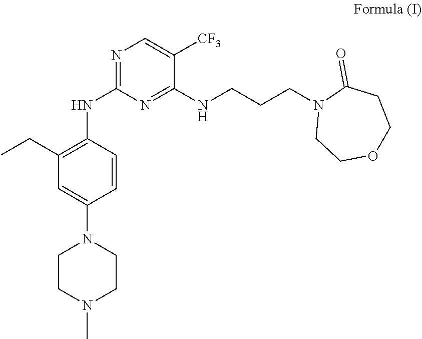 [SMILES: CCc1cc(N2CCN(C)CC2)ccc1Nc1ncc(C(F)(F)F)c(NCCCN2CCOCCC2=O)n1]
or a pharmaceutically acceptable salt thereof; and
(ii) orally administering to the patient a therapeutically effective amount of a KRAS G12C inhibitor.

36. The method of claim 35, comprising administering to the patient about 100 mg to about 600 mg, once daily, of the compound or pharmaceutically acceptable salt thereof.

37. The method of claim 35, comprising administering to the patient about 200 mg to about 600 mg, once daily, of the compound or pharmaceutically acceptable salt thereof.

38. The method of claim 35, wherein the cancer is non-small cell lung cancer.

39. The method of claim 38, wherein the non-small cell lung cancer has a KRAS G12C mutation.

40. The method of claim 38, wherein the non-small cell lung cancer is a KRAS G12C-mutated locally advanced or metastatic non-small cell lung cancer.

41. The method of claim 35, wherein the KRAS G12C inhibitior is sotorasib.

42-62. (canceled)

63. A method of treating an advanced gastrointestinal stromal tumor in a patient in need thereof, comprising:
(i) orally administering to the patient a therapeutically effective amount of a compound represented by Formula (I): 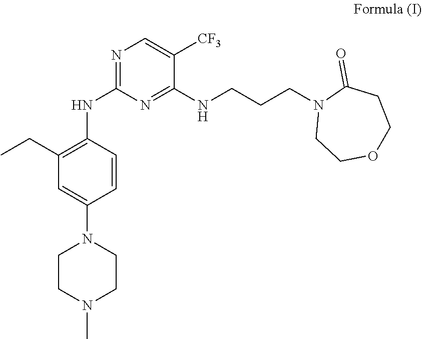 [SMILES: CCc1cc(N2CCN(C)CC2)ccc1Nc1ncc(C(F)(F)F)c(NCCCN2CCOCCC2=O)n1]
or a pharmaceutically acceptable salt thereof; and
(ii) orally administering to the patient a therapeutically effective amount of ripretinib.

64. The method of claim 63, comprising administering to the patient about 20 mg to about 600 mg, once or twice daily, of the compound or pharmaceutically acceptable salt thereof.

65. The method of claim 63, comprising administering to the patient about 20 mg to about 600 mg, once or twice daily, of the compound or pharmaceutically acceptable salt thereof.

66. The method of claim 63, comprising administering to the patient about 100 mg to about 600 mg, once daily, of the compound or pharmaceutically acceptable salt thereof.

67. The method of claim 63, comprising administering to the patient about 150 mg, once daily, of ripretinib.

68. The method of claim 63, comprising administering to the patient about 100 mg, once daily, of ripretinib.

69. The method of claim 63, wherein the gastrointestinal stromal tumor is selected from the group consisting of an NF-1-deficient gastrointestinal stromal tumor, a succinate dehydrogenase (SDH)-deficient gastrointestinal stromal tumor, a KIT driven gastrointestinal stromal tumor, and a PDGFRA driven gastrointestinal stromal tumor.

70-71. (canceled)

72. The method of claim 63, wherein the patient was previously administered at least one tyrosine kinase inhibitors before administration of the ripretinib.

73. The method of claim 63, wherein the patient's gastrointestinal stromal tumor has progressed from, or the patient was intolerant to, a first line administration of imatinib, a second line administration of sunitinib, and a third line administration of regorafenib, or wherein the patient has a documented intolerance to one or more of imatinib, sunitinib and/or regorafenib.
